(19) United States
(12) Patent Application Publication
(10) Pub. No.: US 2017/0360912 A1
(43) Pub. Date: Dec. 21, 2017

(54) CHITOSAN-BASED HYDROGEL AND APPLICATIONS THEREOF.

(71) Applicants: SOCOVAR, L.P., Montreal (CA); VAL-CHUM, LIMITED PARTNERSHIP, Montreal (CA)

(72) Inventors: Sophie Lerouge, Montreal (CA); Elias Assaad, Montreal (CA); Caroline Ceccaldi, Saint Antonin Noble Val (FR); Anne Monette, Montreal (CA); Rejean Lapointe, Laval (CA)

(21) Appl. No.: 15/536,491

(22) PCT Filed: Dec. 17, 2015
(86) PCT No.: PCT/IB2015/059747

§ 371 Date: Jun. 15, 2017

Related U.S. Application Data
(60) Provisional application No. 62/147,231, filed on Apr. 14, 2015.
(60) Provisional application No. 62/092,876, filed on Dec. 17, 2014.

(51) Int. Cl. A61K 39/00; A61K 8/73; A61K 8/98; A61K 9/06; A61K 45/06; A61K 47/36; A61L 31/14; A61L 31/04; A61L 27/54; A61L 27/52; A61L 27/38; A61L 27/20; A61L 27/12; A61K 8/04; A61L 24/00; A61K 49/18; A61K 49/00; A61L 24/08

(52) U.S. Cl. CPC A61K 39/0011; A61L 24/08; A61K 8/736; A61K 8/98; A61K 9/06; A61K 45/06; A61K 47/36; A61L 31/148; A61L 31/145; A61L 31/042; A61L 27/54; A61L 27/52; A61L 27/38; A61L 27/20; A61L 27/12; A61K 8/042; A61L 24/0031; A61L 24/0015; A61K 49/1803; A61K 49/0002; A61K 2800/91; A61K 2039/5158; A61L 2300/416; A61L 2400/06; A61L 2430/02; A61L 2430/36

(57) ABSTRACT

An hydrogel comprising chitosan and two weak bases having different pKb values. In some embodiments, one of the weak bases if sodium hydrogen carbonate (SHC). Also, use of the hydrogel in medical and cosmetic treatments.

### FIELD OF THE INVENTION

The present invention relates to the art of medical and cosmetic treatments. More specifically, the present invention is concerned with a chitosan-based hydrogel and applications thereof.

### BACKGROUND OF THE INVENTION

Chitosan thermosensitive hydrogels have been proposed for multiple biomedical and cosmetic applications during the last decades. However, the simultaneous optimization of gelation time, porosity, mechanical resistance and biocompatibility of hydrogels is still a challenge.

Chitosan (CH) is one of the most abundant polysaccharides of natural origin, mainly obtained from crustaceans. Thanks to its biocompatibility and mucoadhesivity, CH has been proposed for several applications in biomedical, cosmetic, and pharmaceutical fields. However, the applications of unmodified CH have been restrained, because this aminopolysaccharide is soluble only at acidic pH. The neutralization of CH solution with strong bases leads to CH precipitation. A decade ago, a new method was proposed to neutralize CH without inducing precipitation. In fact, the use of the weak base, β-glycerophosphate (BGP), having a pK_{a,2} (6.65 at 25° C.) close to that of CH (about 6.5), allows CH to remain soluble at a neutral pH and to overcome the major obstacle to CH applications. Moreover, this system transforms into hydrogel following heat application, making it suitable as an injectable thermogel. The mechanism of the hydrogel formation is attributable to a heat-induced transfer of protons from CH to glycerol phosphate. The neutralization of CH reduces the repulsive forces among positively charged ammonium groups and allows a stronger interaction of CH chains. As recently reviewed by Zhou, Jiang, Cao, Li, and Chen (2015) CH-BGP thermosensitive hydrogels found numerous biomedical applications. Other studies showed that CH thermogel could also be prepared in the presence of other weak bases, such as ammonium hydrogen phosphate, sodium hydrogen carbonate (NaHCO₃, SHC) or sodium phosphate dibasic (SPD). However, none of these hydrogels was shown to present rapid gelation and high mechanical resistance. High salt concentrations can be used to improve the gelation kinetics, but this increases their cytotoxicity and makes them inappropriate for cell encapsulation (Ahmadi & DeBruijn 2008).

Yet, for many applications, such as drug delivery, tissue engineering or blood vessel embolization, strong but injectable and biocompatible hydrogels are needed. Weak CH hydrogels lead to rapid drug release, do not occlude efficiently blood flow and do not permit to create cohesive scaffolds for cell delivery or tissue engineering. These limitations reduce the potential of the injectable CH hydrogels for pharmaceutical and biomedical applications.

Accordingly, there is a need in the industry to provide a new hydrogel usable in the biomedical and cosmetic fields. An object of the present invention is therefore to provide such a gel.

### SUMMARY OF THE INVENTION

In a broad aspect, the invention provides a chitosan based gel solution which comprises: from about 0.2% to about 4% w/v of chitosan; from about 0.001M to about 0.4M of sodium hydrogen carbonate (SHC), the SHC having a SHC pKb of about 7.65; and a weak base differing from the SHC, the weak base having a weak base pKb. Immediately after preparation, the chitosan based gel solution is flowable and becomes a gel after a gelation time, the gelation time being temperature dependent.

The invention may also provide a chitosan based gel solution wherein the chitosan is chemically modified prior preparation of the chitosan based gel solution to introduce small functional groups to the chitosan structure. Examples of small functional groups include alkyl, carboxymethyl and catechol groups, among others.

The invention may also provide a chitosan based gel solution wherein the weak base pKb is between 3.7 and 7.65.

The invention may also provide a chitosan based gel solution wherein the weak base is selected from the group consisting of phosphate buffer, sodium phosphate dibasic, beta glycerol phosphate, ammonium hydrogen phosphate, sodium acetate and potassium acetate.

The invention may also provide a chitosan based gel solution wherein the SHC is between about 0.04M and 0.1M.

The invention may also provide a chitosan based gel solution wherein the weak base is beta glycerol phosphate at between about 0.001M and about 0.5M.

The invention may also provide a chitosan based gel solution wherein the weak base is phosphate buffer at between about 0.02M and about 0.2M.

The invention may also provide a chitosan based gel solution wherein the chitosan based gel solution includes an acid selected from the group consisting of: hydrochloric acid, acetic acid, propionic acid, citric acid, lactic acid, tartaric acid, malic acid, glycolic acid, ascorbic acid and combinations thereof.

The invention may also provide a chitosan based gel solution wherein the acid has a concentration between 0.0001 M and 0.5M.

The invention may also provide a chitosan based gel solution wherein the chitosan based gel solution has a pH of between about 5 and about 8 prior to gelation.

The invention may also provide a chitosan based gel solution wherein the chitosan based gel solution has a pH of between about 5 and about 8 after gelation.

The invention may also provide a chitosan based gel solution wherein the chitosan based gel solution has physiological pH between about 6.7 and about 7.5 after gelation.

The invention may also provide a chitosan based gel solution wherein the chitosan based gel solution has an osmolality of between 200 mOsm/L and 600 mOsm/L.

The invention may also provide a chitosan based gel solution comprising between about 1.5% and 2% w/v of chitosan.

The invention may also provide a chitosan based gel solution wherein the chitosan has a degree of deacetylation (DDA) of from about 70 to about 100%.

The invention may also provide a chitosan based gel solution wherein the chitosan has a degree of deacetylation (DDA) of from about 85% to about 95%.

The invention may also provide a chitosan based gel solution wherein the gelation time is less than 5 min at 37° C.

The invention may also provide a chitosan based gel solution wherein the chitosan based gel solution remains a free flowing solution for at least 5 minutes after preparation at 20° C.

The invention may also provide a chitosan based gel solution further comprising at least one contrast product for improving contrast in an imaging modality.

The invention may also provide a chitosan based gel solution wherein the at least one contrast product is radiopaque and selected from the group consisting of an iodide-containing solution, iodixanol (Visipaque®), Iopamidol (Isovue®), iohexol (Omnipaque®), diatrizoate meglumine (Conray®), a tantalum powder, a titanium powder and barium sulfate.

The invention may also provide a chitosan based gel solution wherein the contrast product is visible by magnetic resonance imaging and selected from the group consisting of a gadolinium-based contrast agent, gadobenate (MultiHance®), gadoterate (Dotarem®), gadodiamide (Omniscan®), gadopentetate (Magnevist®), gadoteridol (ProHance®), gadoversetamide (OptiMARK®), gadobutrol (Gadavist®) and gadopentetic acid dimeglumine (Magnetol®).

The invention may also provide a chitosan based gel solution further comprising a therapeutic agent for treating a predetermined pathology, the therapeutic agent remaining active for treating the predetermined pathology after gelation.

The invention may also provide a chitosan based gel solution wherein the therapeutic agent is selected from the group consisting of a drug, a bioactive agent, viable cells, and combinations thereof.

The invention may also provide a chitosan based gel solution wherein the therapeutic agent includes a bioactive agent selected from the group consisting of growth factors, fibroblast growth factor (FGF), epidermal growth factor (EGF), Platelet-derived growth factor (PDGF), transforming growth factor-β (TGFβ), insulin-like growth factor (IGF), angiopoietin-1 (ANGPT1), bone morphogenetic protein (BMP)-2, BMP-4, BMP-7, vascular endothelial growth factor (VEGF), platelet-derived growth factor (PDGF), interleukin-1 receptor antagonists, peptide (Link N peptide), anti-cancer drugs, doxorubicin, Irinotecan, antibiotics, doxycycline, or anti-inflammatory drugs, 5-aminosalicylic acid, interleukins (IL), IL2, IL4 and an immune checkpoint inhibitor selected from anti-CTLA-4, anti-PD-1, anti-CD160, anti-CD57, anti-CD244, anti-LAG-3, anti-CD272, anti-KLRG1, anti-CD26, anti-CD39, anti-CD73, anti-CD305, anti-TIGIT, anti-TIM-3, and anti-VISTA.

The invention may also provide a chitosan based gel solution wherein the therapeutic agent includes viable cells selected from the group consisting of stem cells, induced pluripotent stem cells (iPS cells), progenitor cells, differentiated cells, bulge cells, mesenchymal stem cells, endothelial cells, epithelial cells, vascular smooth muscle cells, chondrocytes, osteoblasts, nucleus pulposus cells, CD8 T cells, CD4 T cells, NK cells, gamma delta T cells, B cells, dendritic cells, CAR T cells.

The invention may also provide a chitosan based gel solution wherein the therapeutic agent includes an anticoagulant.

The invention may also provide a chitosan based gel solution wherein the anticoagulant is selected from the group consisting of heparin, dexamethasone and aspirin.

The invention may also provide a chitosan based gel solution wherein the therapeutic agent includes an anti-inflammatory drug.

The invention may also provide a chitosan based gel solution further comprising a mineral cement.

The invention may also provide a chitosan based gel solution wherein the chitosan is combined with another biopolymer excipient.

The invention may also provide a chitosan based gel solution wherein the biopolymer excipient is selected from the group consisting of collagen, chondroitin sulfate (CS), carboxymethylated starch (CMS), hyaluronic acid (HA), Heparin (Hep), extracellular matrix compounds and combinations thereof.

The invention may also provide a chitosan based gel solution further comprising another biopolymer excipient dissolved therein.

The invention may also provide a chitosan based gel solution wherein the chitosan based gel solution reaches, after gelation, a secant storage modulus of from about 10 kPa to about 150 kPa.

In yet another broad aspect, the invention provides a gel obtained through gelation of the chitosan based gel solution as defined hereinabove.

In yet another broad aspect, the invention provides microspheres including the gel as defined hereinabove.

In yet another broad aspect, the invention provides an implant including the gel as defined hereinabove.

In yet another broad aspect, the invention provides a method for manufacturing the implant as defined in claim 36, the method comprising 3D printing the implant by delivering the chitosan based gel solution in free flowing form and heating the thus delivered chitosan based gel solution to form a gel.

Alternatively, the implant may simply manufactured by pouring the chitosan based gel solution in a suitably shaped mold and letting it gel.

In another broad aspect, the invention provides a method for manufacturing the implant as defined hereinabove, the method comprising electrospinning the chitosan based gel solution.

In another broad aspect, the invention provides a delivery system for delivering the chitosan based gel solution as defined hereinabove, the delivery system comprising: a first compartment including an acidic solution of chitosan; a second compartment including an aqueous solution of sodium hydrogen carbonate (SHC) and a weak base differing from the SHC; a mixing compartment in fluid communication with each of the first and second compartment; a delivery element in fluid communication with the mixing compartment for delivering the contents thereof in the human body; and an actuator for simultaneously transferring the contents of the first and second compartments to the delivery element passing through the mixing compartment.

The invention may also provide a delivery system wherein the delivery element includes one of a catheter, a nozzle, and a needle.

The invention may also provide a delivery system wherein the actuator is a main actuator, the delivery system further comprising a third compartment in fluid communication with the delivery element and an auxiliary actuator for delivering the contents of the third compartment to the delivery element.

The invention may also provide a delivery system wherein the third compartment is fillable with a cell suspension.

The invention may also provide a delivery system further comprising a bioactive compound in the third compartment.

The invention may also provide a delivery system wherein the auxiliary and main actuators are usable independently from each other so that the contents of the first and second compartments can be emptied at least partially before the contents of the third compartment is emptied.

In another broad aspect, the invention provides a kit comprising a first container including chitosan; a second container including sodium hydrogen carbonate (SHC), the SHC having a SHC pKb of about 7.65, and a weak base differing from the SHC and; instructions for mixing the contents of the first and second container to obtain the chitosan based gel solution as defined hereinabove.

The invention may also provide a kit wherein the second container includes the SHC and the weak base in powder form.

The invention may also provide a kit wherein the second container includes the SHC and the weak base in an aqueous solution form.

The invention may also provide a kit wherein the first container includes an acidic chitosan solution.

The invention may also provide a kit wherein the first container includes chitosan in powder form.

The invention may also provide a kit further comprising instructions for mixing an effective dose of a bioactive compound to the chitosan based gel solution.

The effective dose includes enough of the bioactive compound to have a predetermined effect on the body of a mammal, for example a human, treated with the bioactive compound.

In yet another broad aspect, the invention provides a method for treating a mammal, the method comprising delivering at a target location in the mammal the chitosan based gel solution as defined hereinabove; and allowing the chitosan based gel solution to gel at the target location after delivery in the mammal.

The invention may also provide a method wherein the chitosan based gel solution is delivered in free flowing aqueous solution form at the target location.

The invention may also provide a method wherein the chitosan based gel solution is delivered using a needle.

The invention may also provide a method wherein the chitosan based gel solution is delivered using a catheter.

The invention may also provide a method wherein the chitosan based gel solution is delivered using a syringe.

The invention may also provide a method wherein the chitosan based gel solution is delivered using a spray nozzle.

The invention may also provide a method wherein the chitosan based gel solution is delivered in a partially gelled form at the target location.

The invention may also provide a method wherein the target location is in a lumen of a blood vessel, the method comprising at least partially embolizing the blood vessel with the gelled chitosan based gel solution.

The invention may also provide a method comprising occluding completely blood flow through the blood vessel with the gelled chitosan based gel solution.

The invention may also provide a method wherein the target location is an abdominal aortic aneurysm in which an endovascular implant has been previously deployed thereby defining an aneurysimal sac, the method comprising at least partially embolizing the aneurysmal sac.

The invention may also provide a method further comprising occluding endoleak areas through transarterial embolization by advancing a microcatheter into the aneurysmal sac through a collateral artery.

The invention may also provide a method wherein the embolization is performed by direct translumbar embolization, the chitosan based gel solution being injected though a needle directly into the aneurysmal sac.

The invention may also provide a method wherein embolization is performed at the time of endovascular aneurysm repair during or immediately after stent-graft deployment.

The invention may also provide a method wherein the chitosan based gel solution includes at least one of mesenchymal stem cells, induced pluripotent stem cells (IPs), vascular smooth muscle cells, fibroblasts and endothelial cells.

The invention may also provide a method wherein the chitosan based gel solution includes at least one bioactive agents selected from the group consisting of growth factors, fibroblast growth factor (FGF), epidermal growth factor (EGF), Platelet-derived growth factor (PDGF), transforming growth factor-β (TGFβ), insulin-like growth factor (IGF), bioactive drug, and doxycycline.

The invention may also provide a method wherein the blood vessel is feeding cancer.

The invention may also provide a method wherein the chitosan based gel solution includes an anti-cancer drug.

The invention may also provide a method wherein the chitosan based gel solution includes at least one bioactive agents selected from the group consisting of bioactive drugs, doxycycline, anti-cancer drug, doxorubicin, Irinotecan, growth factors, fibroblast growth factor (FGF), epidermal growth factor (EGF), Platelet-derived growth factor (PDGF), transforming growth factor-β (TGFβ), and insulin-like growth factor (IGF).

The method as defined in any one of claims 58 to 68, wherein the chitosan based gel solution includes cells selected from the group consisting of stem cells, progenitors or differentiated cells as bone marrow stem cells, induced pluripotent stem cells (IPs), vascular smooth muscle cells, fibroblasts, endothelial cells and combinations thereof.

The invention may also provide a method wherein the target location is in a tissue in need of regeneration.

The invention may also provide a method wherein the target location is a cartilage or an intervertebral disc.

The invention may also provide a method wherein the chitosan based gel solution includes at least one of mesenchymal stem cells, induced pluripotent stem cells (IPs), nucleus pulposus cells, chondrocytes and endothelial cells.

The invention may also provide a method wherein the chitosan based gel solution includes mineral particles promoting bone regeneration.

The invention may also provide a method wherein the mineral particles are selected from the group consisting of hydroxyapatite, calcium phosphates, bioactive glass, clay and silicates and combinations thereof.

The invention may also provide a method wherein the chitosan based gel solution includes viable cells selected from the group consisting of bone marrow stromal cells, induced pluripotent stem cells (IPs), osteoblasts and endothelial cells or progenitor cells.

The method as defined in any one of claims 70 to 75, wherein the chitosan based gel solution includes at least one bioactive agent.

The invention may also provide a method wherein the bioactive agent is selected from the group consisting of a growth factor, a peptide, a drug, a bone morphogenetic protein (BMP), BMP-2, BMP-4, BMP-7, transforming growth factor-β (TGF-B), vascular endothelial growth factor (VEGF), platelet-derived growth factor (PDGF), fibroblast growth factor (FGF), insulin-like growth factor (IGF), and Link N peptide.

The invention may also provide a method further comprising mixing blood obtained from the mammal in the chitosan based gel solution before delivery thereof.

The method as defined in claim 51, wherein the target location in between two tissues to keep separated for a predetermined duration after performing a surgical procedure.

The invention may also provide a method wherein the surgical procedure is laparotomy, thoracotomy or coelioscopy.

The invention may also provide a method wherein the chitosan based gel solution includes at least one of an anti-inflammatory drug, an anticoagulant, heparin, dexamethasone and a molecule having non fouling properties.

The invention may also provide a method wherein the target location is in proximity of or inside a cancerous tumour, the chitosan based gel solution including at least one anti-cancer agent.

The invention may also provide a method wherein the anti-cancer agent includes an anti-cancer drug.

The invention may also provide a method wherein the anti-cancer drug is selected from the group consisting of doxorubicin, Paclitaxel, doxorubicin, Epirubicin, cisplatin, 5-fluorouracil and Irinotecan and combinations thereof.

The invention may also provide a method wherein the chitosan based gel solution is used for immunotherapy.

The invention may also provide a method wherein the chitosan based gel solution includes immune cells, immune checkpoint inhibitors, cytokines or combinations thereof.

The method as defined in claim 86, wherein the immune cells are selected from the group consisting of T lymphocytes (CD8 T cells, CD4 T cells, CAR T cells), B lymphocytes, Natural killer (NK) cells, dendritic cells, immune cells forming tertiary artificial tertiary lymphoid structures (TLS) and combinations thereof.

The invention may also provide a method wherein the immune cells include autologous cells, genetically-modified cells, or both autologous and genetically-modified cells.

The invention may also provide a method wherein the immune checkpoint inhibitors are selected from the group consisting of anti-Cytotoxic T-Lymphocyte Antigen 4 (anti-CTLA-4, Ipilimumab), anti-Programmed cell death-1 (anti-PD-1, nivolumab, pembrolizumab), anti-CD160, anti-CD57, anti-CD244, anti-LAG-3, anti-CD272, anti-KLRG1, anti-CD26, anti-CD39, anti-CD73, anti-CD305, anti-TIGIT, anti-TIM-3, and anti-VISTA antibodies.

The invention may also provide a method wherein the chitosan based gel solution includes at least one of a protein and an oncolytic virus.

The invention may also provide a method wherein the cytokine is interleukine-2 or interleukine-4, the protein is CD40L or the oncolytic virus is the Maraba virus.

The method as defined in claim 51, wherein the target location is at a surface of a wound, delivering at a target location in the mammal the chitosan based gel solution including deposing the chitosan based gel solution on the surface of the wound.

The method as defined in claim 51, wherein the target location is at the surface of a wound, delivering at a target location in the mammal the chitosan based gel solution including spraying the chitosan based gel solution on the surface of the wound.

The invention may also provide a method wherein the wound is selected from the group consisting of a burn, a diabetic ulcer and bullosa epidermolysis.

After gelation, the proposed hydrogel may present a physical barrier protecting the wound. The proposed hydrogel possesses, in some embodiments, a chemical structure close to that of extracellular matrix of tissue and can be uses for tissue protection of wound and help for healing. For this application, the hydrogel can be prepared at room temperature and deposed trough a needle or a sprayer on the surface of the wound. In this application, the hydrogels can be combined with drugs and/or stem cells to treat genetic disease like bullosa epidermolysis.

The invention may also provide a method wherein the chitosan based gel solution includes Mesenchymal stem cells, stromal stem cells, bulge cells, fibroblasts, embryonic stem cells, epithelial cells.

The invention may also provide a method wherein the chitosan based gel solution includes growth factors (fibroblast growth factor (FGF), vascular endothelial growth factor (VEGF), epidermal growth factor (EGF)), angiopoietin-1, interleukin-1 receptor antagonists.

The invention may also provide a method wherein the target location is in or adjacent the intestinal system or in or adjacent the urinary system, the method comprising injecting the chitosan based gel solution to provide mechanical support or treat incontinence.

The invention may also provide a method wherein injection is performed under local anaesthesia.

The invention may also provide a method wherein the incontinence is faecal incontinence, the method comprising injecting the chitosan based gel solution into at least one of an intersphincteric space and an internal anal sphincter to augment the internal anal sphincter and at least partially restore anal sphincter function.

The invention may also provide a method further comprising guiding the injection by monitoring the position of a needle used for injection with guidance from an endoanal ultrasound.

The invention may also provide a method wherein the incontinence is urinary incontinence, the method comprising injecting the chitosan based gel solution as an injectable bulking agent to reconstruct defective periurethral tissue.

The invention may also provide a method wherein the chitosan based gel solution is injected through one of a trans-urethral approach or a periurethral approach.

A method for treating bladder cancer or interstitial cystitis, comprising delivering into the bladder the microspheres of claim 35 in which a therapeutic agent has been incorporated.

Therapeutic agents are agents, such as small molecules, polymers, large molecules, cells, inorganic materials, genetic material and any other agent that can prevent, reduce the incidence, treat, partially treat or reduce the symptoms of a dysfunctions, such as a disease, trauma and degenerative conditions, among others.

The invention may also provide a method wherein the microspheres are delivered through injection through the urethra by cytoscopy.

The invention may also provide a method wherein the therapeutic agent includes at least one of an anti-cancer drug and an anti-ulceric drug.

The invention may also provide a method wherein the anti-cancer drug is doxorubicin, Interleukin-12 or Bacillus Calmettee Guèrin or the anti-ulceric drug is misoprostol or 5-aminosalicylic acid.

The invention may also provide a method wherein the mammal is human.

In yet another broad aspect, the invention provides a cosmetic method for changing the appearance of a human body, the method comprising delivering at a target location of the human body the chitosan based gel solution as defined in any one of claims 1 to 33 in free flowing aqueous solution form; and allowing the chitosan based gel solution to form a gel at the target location.

The invention may also provide a cosmetic method wherein the target location is sub-cutaneous substantially adjacent a wrinkle, the method comprising delivering a sufficient quantity of the chitosan based gel solution at the target location to reduce a volume of the wrinkle.

The non-mammal origin of chitosan (compared with collagen) and its low rate of degradation after implantation (compared with hyaluronic acid) are particularly interesting for this application.

The invention may also provide a cosmetic method wherein the target location is on skin surface, the method comprising covering the target location with the chitosan based gel solution.

The invention may also provide a cosmetic method wherein the chitosan based gel solution includes at least one of hyaluronic acid, collagen, mucopolysaccharides and a naturally occurring polymer.

The invention may also provide a cosmetic method wherein the target location is on a surface of a mucosa.

The invention may also provide a cosmetic method wherein the target location is at a defect in bone or cartilage, the method comprising injecting the chitosan based gel solution in the defect to improve the esthetics of the human body.

Defects can be naturally occurring or may have been caused by trauma or surgery.

The invention may also provide a cosmetic method wherein the defect is in the face of the human body.

The invention may also provide a cosmetic method wherein the chitosan based gel solution includes at least one of mineral cement, a synthetic polymer, a naturally occurring polymer, differentiated cells, precursors cells and stem cells.

In yet another broad aspect, the invention provides a chitosan based gel solution which comprises: chitosan; a first weak base having a first pKb; and a second weak base having a second pKb different from the first pKb; wherein immediately after preparation, the chitosan based gel solution is flowable and becomes a gel after a gelation time, the gelation time being temperature dependent.

The invention may also provide a chisotan based gel solution wherein the first weak base is SHC, the SHC having a pKb of 7.65, and wherein the second pkb is between 3.7 and 11.85.

The invention may also provide a chisotan based gel solution wherein the weak base is phosphate buffer at between about 0.02M and about 0.2M.

The invention may also provide a chisotan based gel solution wherein phosphate buffer is prepared to obtain a pH between 7 and 8.5.

In yet another broad aspect, the invention provides an injectable scaffold containing immune cells for local cancer immunotherapy. The injectable scaffold may further comprise a bioactive molecule.

The present invention proposes numerous thermosensitive hydrogels, solutions for forming the hydrogels and methods of using the hydrogels. The combination of at least two weak bases used as gelation agents provides a solution to the challenges mentioned hereinabove. In particular, using sodium hydrogen carbonate (SHC) as one of the bases allows to obtain injectable hydrogels with quick gelation, high mechanical properties (high storage modulus, G′, high Young modulus in compression, E, and high resistance in compression) despite using relatively low concentrations of salts which make them compatible with cell encapsulation. Hydrogels with physiological pH can be made by choosing appropriate gelling agent and concentration. In particular phosphate buffer at pH7 or 8 allows the formation of hydrogels with pH of about 7 to 7.4, although other pH values are within the scope of the present invention.

When present, therapeutic agents can be added after mixing the chitosan acidic solution with the gelling agent solution, (i.e. when the gel solution is still liquid), or before, in either the chitosan solution or the gelling agent solution and thus enable mixing with other ingredients (cell suspension, particulates, bioactive agents . . . ) before gelation. When the proposed gel solution is at near physiological pH and osmolarity, many therapeutic agents that are unusable when mixed with other gels can be used as these conditions are those that are met by the therapeutic agent when in solution.

In hydrogels targeted for cell therapy and tissue engineering, the injectability, sensitivity to temperature and physiological osmolality of some embodiments of the proposed gel are very useful for cell survival and homogenous encapsulation. Moreover the high mechanical resistance of the proposed gel may be advantageous to provide a scaffold that resists to handling and in vivo stress.

In some embodiments, several properties of the hydrogel present clear advantages for catheter based applications. First, the gel may present low viscosity at room temperature and rheological properties may be kept relatively stable for a relatively long time, even more than 1 hour at room temperature, at least for some formulations, but rapid gelation and good mechanical properties are obtained at 37° C. The gel solution can be injected by small diameter catheter, even several minutes after mixing at room temperature. Also, the composition of the gel and the delay before injection can be chosen to adapt the gel to the clinical need, which can vary depending on low flow or high flow malformations. Its ability to rapidly block pressurized liquid up to physiological pressure (220 mmHg) was demonstrated in vitro. There is little or no risk of catheter gluing, which permit to reuse the catheter after use (after one injection, make another one). The gel can be made radiopaque or MRI visible for imaging control during injection.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

FIG. 1. Storage modulus (G′) of CH hydrogels at various times (0, 3, 30 and 60 min) at 37° C. Data are presented as means±SD (n=3). The results at t0, t3, t30 and t60 of each sample were compared respectively to those of CH:BGP04 (*p<0.05);

FIG. 2. Thermosensitive properties of hydrogels: a) Storage modulus (G′) of CH hydrogels at 22° C. after various times (0, 3, 30 and 60 min) (n=3, mean±SD); b-d) Examples of storage modulus (G′) and loss modulus (G″) variation as a function of time at 37° C. (G′, G″) and 22° C. (G′, G″) (n=3); e-f) Temperature dependence of storage and loss moduli (G′, G″) of CH hydrogels upon heating from 5 to 65° C. at a rate of 1° C./min. Note the different logarithmic scale between e and f (n=3, mean±SD);

FIG. 3. Variation of the secant Young modulus in compression at 5-50% deformation (100% deformation/min) for various CH hydrogels incubated at 37° C. for: a) 1 hour or b) 24 h. Data are presented as means±SD (n=3). Hydrogels prepared with only one gelling agent were too weak to be handled after only 1 hour of gelation;

FIG. 4. Schematic representation of the variation of the secant Young modulus in compression (measured at 50% deformation) of CH hydrogels incubated at 37° C. for 24 hours, as a function of concentration and type of gelation agent;

FIG. 5. FTIR spectra of CH samples and of some selected CH hydrogels (washed and unwashed). Red arrows indicate modifications that may be due to incomplete deprotonation of CH;

FIG. 6. SEM images on cross-sections of some freeze-dried CH hydrogels;

FIG. 7. Viability of L929 fibroblasts exposed to extracts recovered at days 1, 2 and 3 during incubation with various CH hydrogels. (Ctl+=cells in medium culture, Ctl−=cells exposed to 10% DMSO). The data are presented as means±SD (n=16); *p<0.05 compared to Ctl+;

FIG. 8: Osmolality of hydrogels as a function of the final gelling agent concentration, prepared with culture medium as described in 2.2.4. A) Gels with PB=0.04M and increasing concentration of SHC; B) Gels with SHC=0.05 and increasing concentration of PB; C) Gels with increasing concentration of BGP;

FIG. 9: Osmolality of hydrogel as a function of NaCl addition;

FIG. 10. Thermosensitive properties of hydrogels. A) Evolution of the storage modulus G′ during temperature ramps (5 to 65° C. at 1° C./min); B) Evolution of G′ during isotherm at 37° C. (*p<0.05 compared with BGP04 at the same timepoint); C) Evolution of G′ during isotherm at 22° C. (*p<0.05 compared to their own initial G′ value);

FIG. 11: Injectability of chitosan thermogels. A) Maximal force needed to extrude hydrogels from the catheter immersed for 5 min. in saline at 37° C. (*p<0.05); B) Aspect of extruded PB004:SH00075 and PB008:SH0005 (arrows point discontinuities in the PB0.08:SHC0.05 hydrogels after extrusion);

FIG. 12. Mechanical properties of hydrogels after 24 hours of gelation. A) Typical stress-deformation curves of hydrogels. Data were recorded under unconfined compression; B) Secant modulus measured at 50% of deformation of all formulations (mean of n=3 experiments) compared to BGP0.4 (*p<0.05);

FIG. 13: Mechanical properties in tension of various SHC-PB and SHC-BGP hydrogels;

FIG. 14. Scanning electron microscopy images of cross section of hydrogels after 24 hours of gelation. Scale bar corresponds to 200 μm;

FIG. 15. Cytocompatibility of chitosan hydrogels on encapsulated cells. A) Metabolic activity of encapsulated L929 fibroblasts cells for 1 week. ANOVA analysis was performed to compare *) at day 1, metabolic activity of new formulations with BGP0.4 hydrogels and &) for each formulation, day 3 and day 7 to day 1 (*, & p<0.05); B) Metabolic activity of encapsulated human mesenchymal stem cells for 1 week;

FIG. 16: Porosity, mechanical properties, and injectability of CTGels. Rheological properties of hydrogels, where (a) represents the evolution of storage modulus and loss modulus of SHC 0.075 M (CTGel2) at 22° C. and 37° C. and (b) the evolution of storage moduli of the three thermogels at 37° C. (c) Stress-strain curves of thermogels submitted to compression until 50% of strain, after 24 h of gelation at 37° C. (d) SEM images of the ultrastructure of the three CTGel formulations after freeze-drying. (e) Macroscopic observations of CTGel2, 10 min post intra-peritoneal and subcutaneous injections into rats. Results are representative of three independent experiments performed in triplicate;

FIG. 17. Effect of CTGels on T cell viability. (a) Method of cell encapsulation and culture into CTGels. (b) Contour plots of live/dead assays performed using flow cytometry to analyze cells extracted from the different CTGel formulations. (c) Percentage of cells that were alive in the media and the CTGel at the time of extraction from different formulations at day 5 post-T cell encapsulation. [symbol] [symbol] Results are representative of four independent experiments performed in triplicate using three different T cell donors. Graph is representative of average±s.d. ***P<0.001, ****P<0.0001 determined by two-way ANOVA with Tukey's post-test;

FIG. 18. The CTGel formulation promotes cell viability and proliferation of T lymphocytes. (a) Flow cytometry generated scatter plot of live lymphocytes extracted from the CTGel2 formulation over 15 day time course, and (b) logarithmic scale of average cell numbers obtained at different time points from the supernatant and from CTGels 1 or 2. (c) Macroscopic images of CTGels 1 and 2 formulations at day 15 post-cell encapsulation (d) Live/dead assay of CTGel 1- or 2-encapsulated cells over 15 day time course experiments (green, alive; red, dead), and (e) accompanying graph to represent average colony diameters over time. For fluorescence experiments, results are representative of five independent experiments performed in triplicate using three different T cell donors. For flow cytometry, results are representative of four independent experiments performed in triplicate using three different T cell donors. Graphs are representative of average±s.d. ***P<0.001, ****P<0.0001 determined by two-way (b) or one-way (e) ANOVA with Tukey's post-test;

FIG. 19. Ambient conditions surrounding the CTGel influence encapsulated T cell activation. (a) Left to right illustration of gating method of flow cytometry data using FlowJo software contour plots for morphology, live/dead staining, and CD3/CD25. (b) FlowJo generated graph (left) of normalized overlays generated by FlowJo showing CD25 expression of CTGel2 extracted T cells at different days post-encapsulation. [symbol] [symbol] Graph (right) represents percentages of CD3+ CD25+ T cells subsets extracted from the medium and from the CTGel2 at indicated times post-encapsulation, with fresh IL-2 containing medium given at day 8. Results are representative of three independent experiments performed in triplicate using three different normal T cell donors. Bar graphs are representative of average±s.d. **P<0.01, ***P<0.001 determined by one-way ANOVA with Tukey's post-test;

FIG. 20. The chitosan-thermogel favors the growth of CD8+ T lymphocytes. (a) Left to right illustration of part of gating method of flow cytometry data using FlowJo software histogram for CD3+ population gating (left) after morphology/singlets/alive (not shown), for subgating of CD8+ and CD4+ populations on right using contour plots. (b) Averaging graph of percent of CD3+ cells that are also CD8+ or CD4+ was determined from cells collected from the media or those extracted from CTGel2 over a 15-day time course. Bar graphs on right hand side represents phenotypes from control T cells grown media with no presence of CTGel2 at day 8. Experiments were repeated three times in triplicate using three different T cell donors. Bar graphs are representative of average±s.d. *P<0.05, **P<0.01, ***P<0.001 determined by two-way ANOVA with Tukey's post-test;

FIG. 21. T cells and TIL escape the CTGel2 over time. (a) Cumulative graph of average numbers of cells escaping CTGel2 over a 24-day time course for T cells from normal donors (dn.), and for TIL expanded from a ccRCC kidney tumour (k. pt. TIL#1). (b) [symbol] Graph representing percentages of CD3+ T cells that are also positive for CD8+, CD4+ and CD25+ from cells collected from the medium over a 24-day time course experiments. Results are representative of experiments performed in triplicate;

FIG. 22. Renal cancer tumor TIL encapsulated in CTGel2 proliferate and are activated in response to tumor fragments. (a) Depiction of transwell system and legend for all panels in figure. (b) CFSE MFI normalized histogram, changes in CFSE MFI, and changes in numbers of CFSE positive TIL in response to tumor fragments. (c) Live/dead flow cytometry dot plot demonstrating increased number of live TIL in response to tumor fragments (left) with averaging graph (right). (d) Increased numbers of TIL in response to tumor fragments. Numbers of TIL positive (e), and TIL MFI (f) for activation and cytotoxicity markers (CD25, HLA-DR, GZMB, respectively), with averaging graphs below. med: cells from medium; gel: cells from gel; +frag: +tumor fragments in bottom well; k. pt. TIL, ccRCC kidney cancer patient tumor expanded TIL. Graphs are representative of avg.±s.d. *P<0.05, **P<0.01, ***P<0.001 determined by one-way (a-c) or two-way (d-e) ANOVA with Tukey's post-test;

FIG. 23. CTGel2-transwell-mediated recognition/killing assay using α-gp100 T cell clone and melanoma cancer cell lines. (a) Depiction of transwell system, illustrating how α-gp100 T cell clones were encapsulated in CTGel2 within transwells and CFSE labeled cancer cell lines were seeded at the bottom within the culture dish wells. Five days later, cells are collected from the medium at the bottoms of culture dish wells, and were split into two fractions for analysis. (b) Fraction one was analyzed using flow cytometry with gating strategy: lymphocytes (morphology)/singlets/alive/CD8, and then either CD25, PRF1, TNF-α or GZMB. (c) Fraction two was analyzed using gating strategy: cancer cells (morphology)/singlets/CFSE+/AnnexinV. (b-c) FlowJo was used to generate normalized to mode displays of MFIs. (d) Percent cells expressing markers of interest are depicted in averaging graphs representative of two independent experiments preformed in triplicate. (e) Left, ELISA was used to assess the concentration of IFN-γ from the cell supernatants; right, legend provided for all figure panels. Bar graphs are representative of avg.±s.d. *P<0.05, **P<0.01, ***P<0.001 determined by two-way ANOVA with Tukey's post-test;

FIG. 24. Effect of CS concentrations on the mechanical properties (storage modulus G′) of hydrogels as measured with ElastoSens (Rheolution Inc.);

FIG. 25. Growth of L929 encapsulated in various hydrogels (SHC-BGP and SHC-PB gels with 1% CS) as assessed by Alamar Blue fluorescence assay. BGP0.2 and 0.4M and agar gels are shown by comparison;

FIG. 26. Rheological properties of CH—CS hydrogels compared to their CH counterparts (G′ values after 15, 300, 1800 and 3600 s) (Anton Paar MCR301);

FIG. 27. Rheological properties of CH-heparin hydrogels compared to their CH counterparts during gelation at 37° C. (Anton Paar MCR301);

FIG. 28. Heparin release from CH-heparin hydrogels prepared with SHC0.075PB0.04M or SHC0.075BGP0.1 gelling agent solution;

FIG. 29: X ray imaging of a) CH-SHC-PB gels with 0, 30, 50% v/v Visipaque and saline solution as comparison; b) CH-SHC-PB gels with 50% v/v Visipaque after 0, 1, 4 and 24 h immersion in a saline solution;

FIG. 30: IRM 3T in a) T1 and b) T2 sequence of hydrogels prepared with increasing concentration of Multihance [0.01%, 0.1%, 1% et 10%] The lines correspond to CH gels prepared without Visipaque or with Visipaque (30% or 50%);

FIG. 31: Influence of addition of Visipaque (radiopaque contrast agent) with and without Multihance (Gadolinium-based contrast product) on gelation kinetic and rheological properties during the first 10 min of gelation (mean+SD; n=3);

FIG. 32. Evolution of the storage modulus (G′) of chitosan hydrogels (2% w/v CH) mixed with different gelation agents with and without DOX (0.1% and 1% w/v) and Visipaque contrast agent (30% and 50% v/v) at 37° C., as a function of time;

FIG. 33. Percentage of DOX release from a) CH-SHC075PB08-DOX b) CH-BGP04-DOX gels after 60 hours by dissolution test;

FIG. 34. Maximum flow pressure sustained by CH gels with and without DOX and VISI evaluated by embolization bench test;

FIG. 35. Factor VIII immunostaining of ex vivo embolized aortic vessels: (a) untreated; (b) embolized with CH; (c) embolized with CH/DOX 0.1%; (d) embolized with CH/DOX 0.3%; (e) embolized with CH/DOX 1%. The figure shows endothelial ablation after embolization with all CH/DOX0.1%, 0.3% and 1%. Arrows shows the positive stain corresponding to endothelial cells;

FIG. 36, Illustration of endovascular treatment of abdominal aortic aneurysms, where the hydrogel can be injected in the aneurysmal sac to treat or prevent endoleaks;

FIG. 37. Porcine kidney under Digital substraction Angiography (DSA) a) before embolisation; b) partial embolization via injection of CH/VIS50%-DOX0.1% gels in the renal artery;

FIG. 38. Preliminary assay of prevention of tissue Adhesion by injectable chitosan hydrogel;

FIG. 39: Biodegradation of hydrogels: Evolution of storage modulus during immersion in lyzozyme;

FIG. 40. Rheological properties of hydroxyapatite-containing CH gels; and

FIG. 41, in a schematic view, illustrates a delivery system in accordance with an embodiment of the present invention.

### BRIEF DESCRIPTION OF THE TABLES

The following tables, shown in the drawings pages, are referred to in the detailed description hereinbelow.

Table 1. Abbreviations and details of composition of the different hydrogels tested. The initial concentrations of CH (before mixing CH and gelling agent) and the final concentrations (in the hydrogel) were 3.33 and 2% w/v, respectively. Phosphate buffer solutions (PB) of pH 7 or 8 were used.

Table 2. pHs of hydrogels immediately after mixing and of hydrogel filtrates.

Table 3: Composition, pH, and osmolality of chitosan hydrogels (Example 2).

Table 4: Formulations and physico-chemical properties of chitosan thermogels (CTGels) (Example 4).

### DETAILED DESCRIPTION

In the present document, the term “about” is used to describe variations in numerical value that may be due to measurement errors, measurement uncertainties or manipulation errors, but which have no significant effect on the way the claimed invention works. Also, the present application claims priority from U.S. provisional patent applications 62/092,876 filed Dec. 17, 2014 and 62/147,231 filed Apr. 14, 2015, the contents of which is hereby incorporated by reference in their entirety.

The present invention proposes numerous thermosensitive hydrogels, solutions for forming the hydrogels and methods of using the hydrogels. The combination of at least two weak bases used as gelation agents for chitosan provides a solution to the challenges mentioned hereinabove. In particular, using sodium hydrogen carbonate (SHC) as one of the bases allows to obtain injectable hydrogels with quick gelation, high mechanical properties (high storage modulus, G′, high Young modulus in compression, E, and high resistance in compression), despite using relatively low concentrations of salts which make them compatible with cell encapsulation. Hydrogels with physiological pH can be made by choosing appropriate gelling agents and concentrations. In particular phosphate buffer at pH 7 or 8 allows the formation of hydrogels with pH of about 7 to 7.4, although other pH values are within the scope of the present invention. The concentration of salt can also be chosen and adjusted to obtain isotonic hydrogels. Moreover the concentration and ratio of the two bases enable to adjust the gelation rate, mechanical properties and macroporosity according to the need for a particular application.

Overall, these new hydrogels provide interesting alternatives for use alone or in combination with cells, bioactive agents or drugs for the treatment of pathologies or for the repair or engineering of new tissues. In particular they are interesting as embolizing agents of blood vessels, or as injectable scaffolds for drug delivery and/or cell seeding in tissue engineering strategies. In addition, the proposed hydrogel has potential for use in cancer treatment in which immune cells could be contained in the proposed gel that is then injected at a targeted treatment site. The proposed gel may also be used in bioactive substance controlled release, such as anti-cancer molecules, in which the substance to be released is contained in the gel. The gel may also contain bioactive agents and can then be applied on the skin or mucous surfaces. These hydrogels also provide large potential for bioprinting technologies, where complex 3D constructs are created which contains cells, since they provide a highly hydrated and biocompatible environment but mechanical properties that enable to handle the constructs. Many other possible applications for the proposed hydrogel have been enumerated hereinabove, and some are further described hereinbelow. Also, it should be noted that the present hydrogel and solutions for forming the hydrogel are not limited to these examplary uses.

The proposed hydrogels have very interesting properties that can be optimized depending on the needs by changing the concentration and relative ratio of the two gelling agents (SHC and weak base) and the pH of the solution. Their mechanical properties after gelation are largely superior to those of chitosan-BGP gels, and higher than chitosan cross-linked with genipin. They are injectable but when heated they may exhibit high gelation rate and strong immediate resistance, which is drastically superior to CH-BGP gels. This permits, for example, to provide cohesion once injected and to occlude blood vessel. These gels can be made radiopaque or MRI visible by adding a contrast agent, while keeping rapid gelation. Gelation rate can be controlled by modifying the pH of the phosphate buffer and the respective concentrations of the two gelling agents. Also, they may present low salt concentration leading to physiological osmolality (which can be adjusted by addition of NaCl or any other suitable salt if necessary) and good cell compatibility. In contrast, CH-BGP gels with rapid gelation require high BGP concentration, which make the gel noncompatible with cell seeding due to high ionic strength. The new hydrogels form porous matrices with various pore sizes, depending on their composition. In addition, they are thermosensible. Therefore, gelation is absent or slow at low temperature and more rapid when increasing the temperature (so gelation at body temperature is much more rapid than at 6° C. or room temperature). Furthermore, they are biodegradable and their rate of degradation can be modified by changing the DDA (Degree of Deacetylation) of chitosan. Price of ingredients is relatively small and preparation is easy. Even more importantly, the chitosan, a biopolymer of natural origin, may be used without any chemical modification if desired. The gelling agents used are presumed to be biocompatible at low concentrations, since they are present in human body, particularly in blood. In conclusion the present invention provides, in some embodiments, an injectable scaffold of low viscosity at injection (therefore injectable through needles or small diameter catheters), compatible with cells and presenting rapid gelation and high mechanical properties (Young modulus and resistance to compression). Their potential for cell therapy and tissue engineering is high. Indeed such in situ-formed gel, induced by a physiologically permitted stimulus (here temperature), can function as cell scaffold and may be very useful in fabricating a custom-made or complex shaped hybrid tissue in tissue engineering applications. These gels also present interesting advantages for drug delivery.

These advantageous properties of the proposed hydrogel are reached thanks to a synergy between gelling agents. The new hydrogels are prepared by using a combination of at least 2 weak bases of different pKb. For example sodium hydrogen carbonate (SHC) can be combined with phosphate buffer (or simply with sodium Phosphate Dibasic, present in phosphate buffer) or with BGP, as demonstrated by data in examples. Other weak base such as ammonium hydrogen phosphate is also hypothesized to product an acceptable gel. SHC plays a major role in drastic improvement of mechanical resistance and compression strength of the CH hydrogels, probably due to its weak basicity, its low molecular weight and its decomposition in acidic medium into (CO₂+H₂O). SHC alone at some specific concentrations also leads to strong hydrogel but with very slow gelation which make them unsuitable as injectable gels. The combination with another weak base enables to reach also quick gelation. More generally, here, the simultaneous use of two weak bases with different pKb (H₂PO₄²⁻, pKb 6.8 and HCO₃—, pKb 7.65) to neutralize protonated chitosan permits the control of the gelation rate and gelation time of chitosan. Thus, it is hypothesized that the strongest base should react with the protonated chitosan more rapidly than the weakest base. Then, by modifying the ratio of the two bases, the gelation process of chitosan can be delayed or accelerated. This method can be extended to other weak bases able to neutralize protonated chitosan at different rates. Finally, due to its decomposition, SHC may disappear completely from the final hydrogel, without releasing toxic products.

When used for medical or cosmetic treatment, the proposed chitosan based gel solution may be prepared in numerous manners. In a first example, the required products are provided in bulk and measured before being mixed. In another example, kits are provided with just the right quantities of products, in solution or powder form required to form a gel with predetermined properties. In yet other embodiments, the required products are provided in the form of a delivery system.

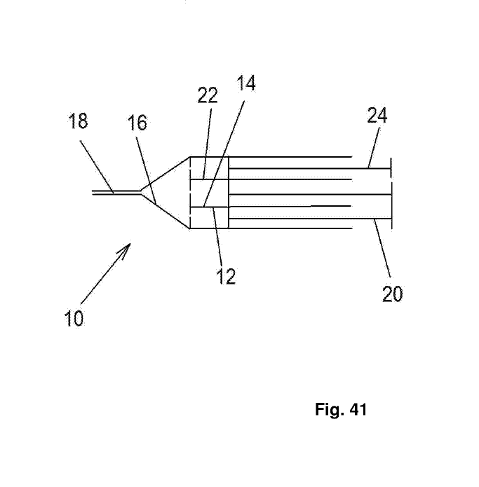

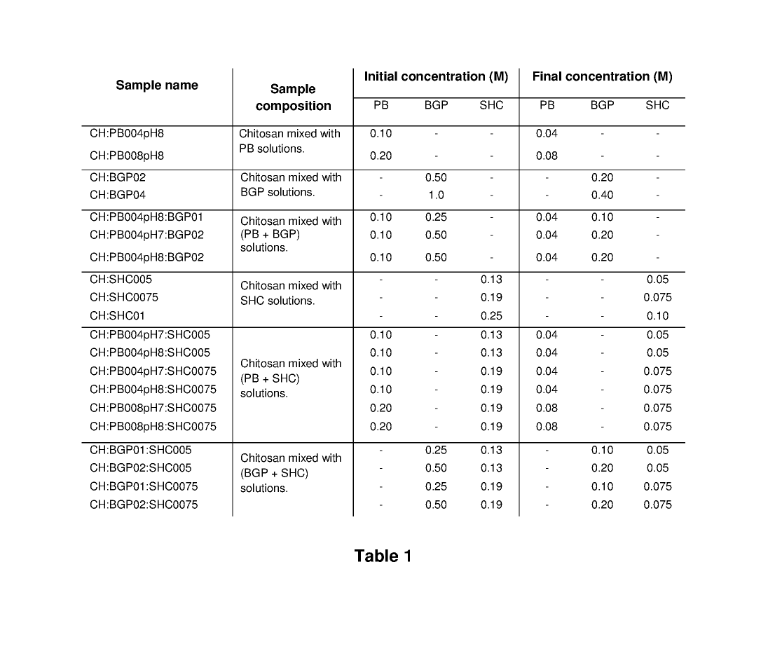

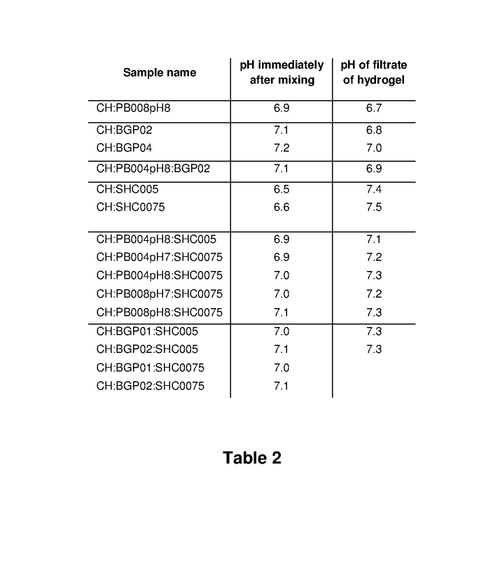

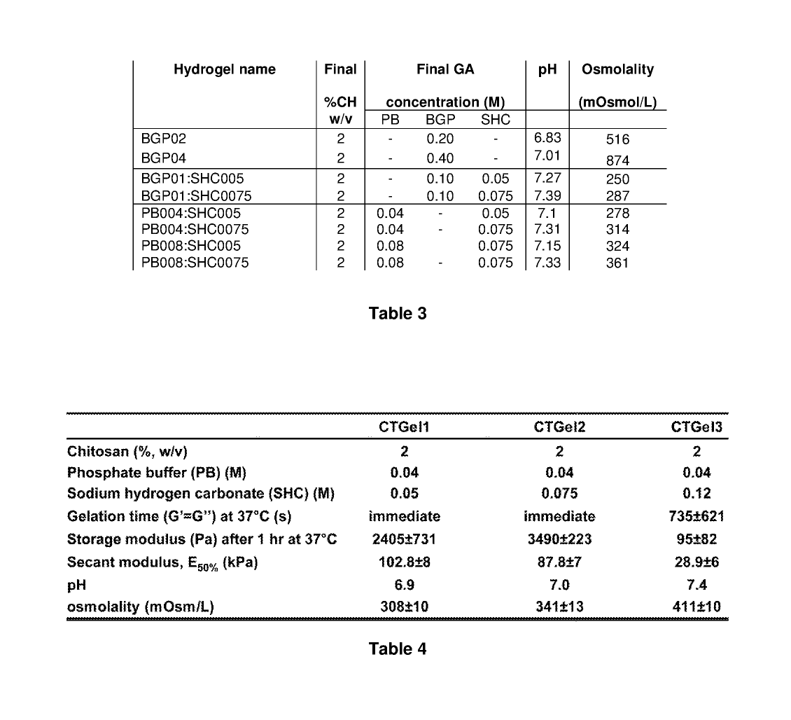

Referring to FIG. 41, there is shown a non-limiting example of such a delivery system 10 for delivering the chitosan based gel solution according to the invention. The delivery system 10 includes a first compartment 12 including chitosan in an acidic solution or in powder form and a second compartment 14 including an aqueous solution or a powder mix of sodium hydrogen carbonate (SHC), the SHC having a SHC pKb of about 7.65, and a weak base having a weak base pKb smaller than the SHC pKb. A mixing compartment 16 is in fluid communication with each of the first and second compartments 12 and 14. A delivery element 18 is in fluid communication with the mixing compartment 16 for delivering the contents thereof in a human body. An actuator 20 is provided for simultaneously transferring the contents of the first and second compartments 12 and 14 to the delivery element 18 passing through the mixing compartment 16. For example, the delivery system 10 includes a conventional multi barrel syringe to which may be connected a delivery element in the form of a nozzle, a needle or catheter. In that case, the first and second compartments 12 and 14 are substantially elongated and parallel to each other and the actuator 20 is a plunger engaging both the first and second compartments and longitudinally movable therealong. When powders are present in the first and second compartments, the delivery system is typically used by first filling the first and second compartments 12 and 14 with a predetermined quantity of water. In some embodiments, the contents of the first and second compartments 12 and 14, and eventually of a third compartment 22 described hereinbelow, can only be transferred to the mixing compartment 18 is pressure within the first, second and third compartments 12, 14 and 22 reaches a predetermined threshold, for example by using a suitable valve that opens upon the threshold being reached. Such valves include for example thin polymer leaflets that are biased towards each other.

As shown in FIG. 41, the delivery system 10 is provided in some embodiments with a third compartment 22 having an auxiliary actuator 24 that can be operated independently from the actuator 20. This allows starting mixing the contents of the first and second compartments 12 and 14 before any of the contents of the third compartment 22 is delivered.

The delivery system 10 may be used in a method for treating a mammal, the method comprising delivering at a target location in the mammal the chitosan based gel solution and allowing the chitosan based gel solution to gel at the target location after delivery in the mammal. In such embodiments, the delivery system 10 is either assembled and filled with the appropriate solutions in the first and second compartments 12 and 14 just prior to use, or the delivery system is provided with the first and second compartments 12 and 14 already filled with the appropriate solutions and the delivery element 18 is secured to the remainder of the delivery system according to the specific use for the delivery system. For example, subcutaneous injections of the chitosan based gel will require a delivery element 18 in the form of a needle. Catheter based methods will require a delivery element 18 in the form of an elongated catheter. In some embodiments, the methods of treatment use a conventional single barrel syringe instead of the above-described delivery system 10. In these embodiments, the chitosan based gel solution is mixed just prior to use and the syringe is filled therewith. Then, the chitosan based gel solution can be delivered as any other liquid used in methods of medical treatment. It should be noted that any other suitable manner of delivering the chitosan based gel solution may be used, such as the numerous conventional methods used in the medical and cosmetic fields.

Preparation of the gel solution takes advantage of the thermosensitive character of the corresponding hydrogel and is prepared at room temperature or below (for example between 6 and 22° C.). For cell encapsulation, gel solution with close to neutral pH and osmolality close to physiological range is chosen.

The hydrogel is usable in some embodiments as an embolizing agent, for example for the embolization of blood vessels (aneurysms, blood vessel malformations or undesirable blood flow), either alone or with cells to help healing or with drugs to help healing or counter negative effects. The hydrogel could be used as an injectable hydrogel or as pre-formed microspheres, among other possibilities. In some embodiments, a contrast agent may be included in the gel. It was shown that Visipaque® contrast agent minimally affected the mechanical properties of the gel, and the same is expected for many other contrast agents.

The hydrogel is also usable as an injectable carrier for a therapeutic agent, a bioactive agent, cells or combinations thereof. As mentioned hereinabove, other applications involve injection in various tissues for various purposes, treatment of various tissue surfaces and delivery in the bladder, blood vessels and other body cavities, among others.

In some embodiments, after delivery, the proposed hydrogel rapidly forms a solid cast and forms a porous scaffold in which cells can grow. The hydrogel will be progressively degraded and replaced by tissue formed by the cells. Several works have already shown the potential of stem cell therapies for improving the outcome of inflammation-based diseases including aortic aneurysms. Indeed mesenchymal stem cells (MSCs) contribute to aortic remodelling. A few studies recently showed potential benefit of cell seeding for AAA treatment. Zhao et al showed that MSC treatment significantly attenuated AAA formation and IL-17 production in elastase-perfused WT mice. MSC implantation was shown to inhibit Ang II-induced AA development in apoE(−/−) mice through elastin preservation in the aortic wall and it was associated with attenuated levels of MMPs and inflammatory cytokines.

VSMCs endovascular seeding was also shown to restore the healing capabilities of proteolytically injured extracellular matrix in aneurysmal aortas, and stops expansion in a model of aortic injury elicited by inflammation and proteolysis. Despite that, to the best of our knowledge, a safe and efficient approach for MSC seeding, in combination or not with stent graft deployment has not been developed before availability of the proposed hydrogel.

In another embodiment, a drug can be added to the gel, with or without the cells. For example doxycycline, an antibiotic which is a known inhibitor of metalloproteinase (MMPs) known to be involved in the progression of aneurysms, can be mixed in the chitosan based gel solution and be released progressively to counter aneurysm progression.

The invention also provides a method of treatment using any of the above mentioned hydrogels injected in a subject, for example a mammal subject, and in a more specific example, a human subject. For example, the gel is injected in a fluid or viscous state at about room temperature and has a composition resulting in gelation inside the body at physiological temperature. The invention also provides a method of treatment using any of the above mentioned hydrogels applied to a free surface of a tissue. Such treatments include for example the fabrication of bandages for cicatrisation of wounds. Such treatments may also be cosmetic.

The chitosan based gel solution can be delivered using conventional catheter-based or needle based methods. For example, in catheter-based methods, a catheter is inserted through a natural opening in the body, such as through the mouth, nose, anus, uretha, vagina and ear canal, percutaneously to reach a blood vessel, or by laparoscopy, until a region of the body in which the gel is to be provided is reached. Guidance can be done conventionally using fluoroscopy. For example, in needle-based method, the needle in inserted percutaneously to the same effect, or the needle may be provided at the end of an instrument inserted in the body as in catheter-based methods. Then, the chitosan based gel solution can be delivered and left to gel.

Occluding blood vessels can also help create voluntary ischemia. An example of the latest case is endovascular treatment of hypertrophic cardiomyopathy. Hypertrophy of the septum can strongly reduce blood ejection from the right ventricular. To reduce hypertrophy, obstruction of the appropriate septal artery is a possible endovascular approach to provoke an artificial ischemia and necrosis of the tissue. The proposed hydrogel may be used for such purposes.

Occlusion of blood vessels can also be used for chemoembolization, for example as palliative treatment of hepatic cancer, where occlusion of the artery reduces access to oxygen and nutrients to cancer and an anti-cancer drug can be added to the occlusive product.

In other embodiments, a fully formed gel is implanted, for example surgically, or injected in the form of microspheres.

In some embodiments, the above mentioned hydrogels are used to form 3D scaffolds containing cells in vitro, for example by bioprinting techniques. Bioprinting techniques are increasingly used to produce complex tissue structures in vitro, for tissue engineering or 3D drug screening tools, for example. In these cases mechanical properties are essential for handling purpose and to conserve the 3D geometry. Low initial viscosity of the gel at room temperature enables to use small diameter systems with high precision while rapid gelation enables to increase the final precision of the geometry. Finally the high water content and isoosmolality protect cells from dehydration and damage. Such hydrogel-based constructs can be used to engineer various tissues, such as skin, blood vessels, intervertebral disk, cartilage and many others. In some embodiment, the hydrogel can be used to occlude different types of arteries, veins, and venous or arteriovenous malformations, to prevent undesirable blood flow.

Several methods can be used for bioprinting: they can be divided into laser-based methods, printer-based methods and nozzle-based methods. In nozzle-based systems, the CH and gelling agent solutions are first mixed, cells are then added, and the solution is put in pressure-assisted syringes to deposit continuous strands of materials according to the defined pattern (defined by CAO or based on 3D reconstruction of images from patients to fit a particular tissue defect). The substrate can be heated to accelerate gelation once injected and keep the encapsulated cells at 37° C. 3D structures containing different cells or various bioactive products can thus be produced. Multiple cell types in sufficient resolution enables to recapitulate biological function. To that issue, syringes containing hydrogels of different composition (various gelation agents, various type of cells, various DDA of chitosan) can be combined or used one after the other to form complex structures of desired (and variable) porosity, cells content etc.

Printer-based systems including thermal and piezoelectric inkjet printing. They generate small droplets of low viscosity ‘bio’ ink that form 3D constructs. The ideal viscosity of the initial solution can differ from one technology to another and can be adjusted by changing the concentration or ratio of each gelling agent and other CH concentration, molecular weight or DDA.

The proposed hydrogels are also good candidates for electrospinning. This process uses an electrical charge to draw very fine (typically on the micro or nano scale) fibres from a liquid. In those cases, the hydrogels can be used with cells, drugs and/or bioactive molecules. The rheology of the solution can be adjusted for example by changing the ratio and concentration of gelation agents. Electrospinning is a process which exploits electric fields between two electrodes to generate fibers and scaffolds towards a grounded or oppositely charged electrode. In this particular embodiment, the hydrogel is put in a syringe at low temperature (between 4 and 25° C. for example) and the potential difference between the electrodes accelerates the charged liquid towards the opposite electrode thereby causing the drawing of continuous fiber. By changing the experiment parameters (voltage, flow rate, media properties etc.), various fiber diameter and alignment can be created.

The CTSgel can be used with cells to treat a variety of tissue defects and regenerate tissues, thanks to its cytocomptability and enhanced mechanical properties, as demonstrated in the present document. For those applications, a variety of cells can be added to the gel prior to injection, including mesenchymal stem cells which are known for their great potential for tissue regeneration and cell therapy.

For example, it can be used to regenerate cartilage in case of joint chondropathies and void filling cartilage defects due to trauma or arthritis. In this case, chondrocytes or stem cells can be encapsulated in the gel before injecting it to the joint through a needle. The gel could be first mixed with some patient blood prior to injection in the defect and can be associated with mineral cements.

Another potential application is its use as a scaffold for the regeneration of intervertebral disk, using appropriate differentiated cell like nucleus pulposus cells and/or stem cells. For these applications, a bioactive molecule can be added.

Prevention of postoperative adhesions, both for human and veterinary applications, is another possible application. In this application, the hydrogel can serve as a temporary biodegradable barrier to make a physical presence and separates adhesiogenic tissue while the normal tissue repair process takes place. In some embodiments, the hydrogel has to stay relatively intact for at least 3 days and then degrade after implantation. The hydrogel can be prepared at room temperature and injected trough a needle or sprayer in laparotomy, thoracotomy or coelioscopy. The gel can be mixed with anti-inflammatory drug or anticoagulants (including heparin, dexamethasone and aspirin) or molecules/polymers having non fouling properties. Stem cells can also be added in the gel for their paracrine activities. For example, mesenchymal stem cells infused in periphery blood have shown to be able to reduce abdominal adhesion score in rats. However, intra-abdominal injection of MSCs could not. This was due to exposition of MSCs to macrophages in intra-abdominal case. The association of MSCs with a scaffold could protect MSCs from phagocytosis.

In other embodiments, the proposed hydrogel may be used as a vehicle for cosmetics. For this application, the hydrogel can be prepared at room temperature and deposed trough a needle or a sprayer on the surface of the wound. In this application, the hydrogels can be combined with hyaluronic acid for example for cosmetic application.

In other embodiments, the proposed hydrogel may be used as a cell delivery vehicle for the growth and release of autologous cells such as immune cells isolated from a patient's tumor (Tumor infiltrating T lymphocytes, TIL) or genetically modified in a laboratory (CAR T cells). One particular formulation has been shown particularly well adapted macroporosity and biocompatibility to the growth of T cell colonies and the progressive release of T cells, which remain active against cancer cells. In addition to T cells, other immune cells and bioactive agent such as immune checkpoint inhibitors could be added to create artificial tertiary lymphoid structures (TLS) and boost the activity of immune cells. Local delivery of this anti-cancer vehicle could thus increase the efficacy and decrease side effects of actual immunotherapies.

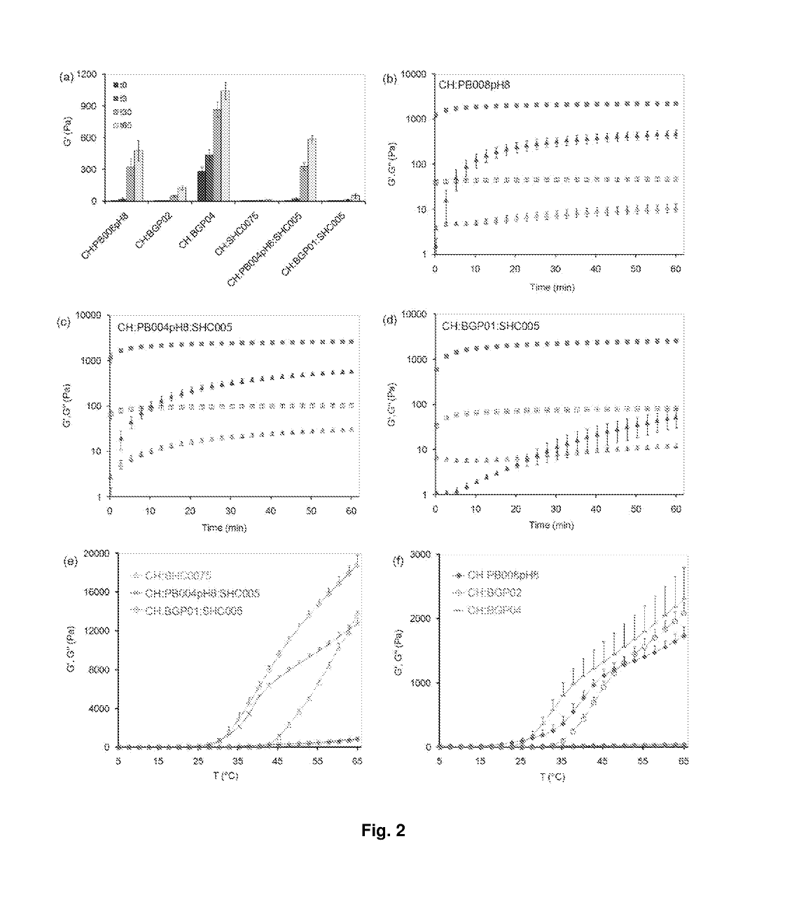

The present document uses a shortened notation to refer to the Figures. More specifically, when the terms “FIG. 2a”, “FIG. 2a” and the like are used, they are a shorthand notation for FIG. 2, panel a.

### Example 1

### Materials and Methods

Shrimp shell chitosan (Kitomer, PSN 326-501, Premium Quality, Mw 250 kDa, DDA 94%) was purchased from Marinard Biotech (Rivière-au-Renard, QC, Canada). β-Glycerol phosphate disodium salt pentahydrate C₃H₇Na₂O₆P.5H₂O (BGP), sodium phosphate monobasic NaH₂PO₄ (SPM) and sodium phosphate dibasic Na₂HPO₄ (SPD) were purchased from Sigma-Aldrich (Oakville, ON, Canada). Sodium hydrogen carbonate NaHCO₃ (SHC) was purchased from MP Biomedicals (Solon, Ohio, USA). The other chemicals were of reagent grade, and were used without further purification.

The chitosan (CH) was purified following the method described by Qian and Glanville, with some modifications. A total of 6 g of raw CH was dissolved in 600 mL of 0.1 M hydrochloric acid by stirring overnight at 40° C. The acidic solution was filtered under vacuum through qualitative grade filter paper (Fisherbrand) to remove insoluble particles. The CH was then precipitated with 0.5 M NaOH under continuous stirring at room temperature. Next, the slurry (pH 8-9) was heated at 95° C. and the stirring was kept for 5 minutes following the addition of 6 mL of sodium dodecyl sulfate 10% w/v. Subsequently, the slurry was cooled down to room temperature, and the pH was adjusted to 10, with 0.5 M NaOH. The slurry was filtrated under vacuum and then the hydrated CH was washed 5 times with 600 mL of Milli-Q water at 40° C. A solution of barium chloride was used to confirm the absence of sodium dodecyl sulfate in the filtrate. Finally, the hydrated CH was freeze-dried, ground and sieved to get the dried and purified CH used for the experiments.

Chitosan hydrogels (2% w/v) were prepared at room temperature by mixing a chitosan acidic solution with a solution containing one of the gelation agent(s), namely β-glycerol phosphate (BGP), sodium hydrogen carbonate (SHC), phosphate buffer (PB) or their combination (SHC:PB or SHC:BGP).

Phosphate buffer solutions (PB) at approximately pH 7 and 8 were prepared in Milli-Q water by dissolving SPM and SPD salts at w/w ratios of 0.540 and 0.047, respectively. The BGP and SHC solutions were prepared by dissolving the corresponding salt in Milli-Q water, while the BGP:PB and SHC:PB solutions were prepared by dissolving salt in PB. The mixture solutions of BGP and SHC were prepared by dissolving the salts of BGP and SHC together in Milli-Q water. For cell experiments, the solutions were sterilized by filtration through 0.22 μm filters.

A CH solution of 3.33% w/v was prepared by dissolving purified CH powder in 0.1 M hydrochloric acid. The stirring was kept overnight at room temperature, and then the solution was sterilized by autoclave at 121° C. for 20 minutes and stored at 4° C. The pH of the CH solution at room temperature (22° C.) was about 6.2.

Each hydrogel was prepared at room temperature by mixing a CH solution with a solution of gelling agent at a ratio of 0.6:0.4 by using two syringes and a female-to-female Luer Lock syringe connector. pH measurements were carried out at room temperature immediately after mixing the two solutions, using a Denver Instrument UltraBasic pH-meter. After 24 h gelation at 37° C., pH was measured again by pH papers.

In the following text, the names of the hydrogels were chosen in a manner allowing the composition of each kind of hydrogel to be known. For example, CH:BGP01:PB004pH7 represents a hydrogel containing 2% w/v CH, 0.1 M BGP and 0.04 M PB at pH 7. The values correspond to the final concentrations in the hydrogel and to the pH of the PB used. All hydrogels were prepared to reach a final concentration of 2% w/v chitosan (Table 1).

The rheological properties of the hydrogels were studied using an Anton Paar instrument (Physica MCR 301, Germany) equipped with a co-axial cylinder geometry (CC10/T200) and connected to a circulating water bath (Julabo AWC100, Germany). The variation with time of storage modulus (G′) and loss modulus (G″) was measured in the linear viscoelastic range, at a constant shear stress (1 Pa) and a constant frequency (1 Hz) immediately following the preparation of each hydrogel (n=3). The measurements were carried out at 22° C. (room temperature) and 37° C. (body temperature) for one hour for each sample of 1.5 mL. To better understand the effect of temperature, some tests were also carried out while increasing the temperature from 5 to 65° C. at the rate of 1° C./min. In the latter case, the solutions were kept at 4° C. before mixing. Sample drying was prevented by adding a few drops of corn oil on the top of the samples. Each test was performed in triplicate.

Axial unconfined compression tests were performed at room temperature using Bose ElectroForce® 3200 instrument (Bose Corporation, USA) equipped with a 22 N load cell, to evaluate the hydrogels strength after 1 or 24 h gelation at 37° C. Samples of 2 mL were prepared (n=3) in small cylindrical containers (14 mm internal diameter; 12 mm height), and then kept in an incubator. The hydrogels were gently pushed out, measured for size and then characterized by applying progressive compression up to 50% (0-0.5) at the rate of 100% deformation/min. The displacement and the load values were used to calculate the Young's secant moduli considered as the slope of a line connecting the point of zero strain to a point at a specified deformation (from 0.05 to 0.50).

The Fourier transform infrared (FTIR) spectra were obtained for several samples: i) CH powder, ii) a freeze-dried sample of completely protonated CH (CH—H⁺), iii) a freeze-dried sample of CH solution used in the preparation of the hydrogels, iv) the salts used to prepare the gelling agents solutions (not showed), v) unwashed freeze-dried hydrogels and vi) washed freeze-dried hydrogels. These later hydrogels were washed as follow. Each hydrogel was crushed and dispersed in 80 mL of Milli-Q water, then filtered after 30 min stirring. These steps were repeated 6 times, then the sample was frozen at −20° C., freeze-dried and ground. FTIR spectra were recorded at wavenumbers from 4000 to 400 cm⁻¹ and at 2 cm⁻¹ resolution with 32 scans using a Nicolet 6700 FTIR spectrometer (ThermoElectron Corporation,). The pellets were prepared by the compression of homogenous mixtures of KBr and powder of each sample in flat-faced punches.

The morphology of the dried hydrogels was examined using a Hitachi S-3600 SEM. The hydrogels (2 mL) were prepared in 24 well plates, in duplicate, and were kept for 24 hours at 37° C. for gelation. The hydogels were then kept at −20° C. overnight and freeze-dried under vacuum for 24 hours. Small pieces of each sample were gently cut, deposited on double-coated carbon conductive tape previously adhered to SEM aluminum stubs, and then sputter-coated with a thin gold layer before analysis.

The cytotoxicity of the hydrogels was evaluated on hydrogel extracts using L929 fibroblast cells (ATCC, Manassas, Va., USA) following ISO 10993-5:2001 standards, with some modifications. Hydrogel extracts were prepared as follows: samples (1 mL each) were left to gel in a 12-well plate for 3 days at 37° C. in an incubator, and then 3 mL of culture medium were added on the top of each hydrogel. At days 1, 2 and 3, the medium was recovered and replaced by a fresh medium.

The cells were seeded in 96 well plates at a concentration of 10⁵ cells per well, and were observed until achieving 80% confluence in normal Dulbecco's Modified Eagle's Medium (Gibco BRL, Invitrogen, Grand Island, N.Y., USA) supplemented with 10% fetal calf serum (FCS; Medicor, Montreal, QC, Canada) and 1% glutamine (PS, Gibco BRL, Invitrogen). The culture medium was then removed and replaced with medium containing the extract. After 24 hours, the cells were exposed to Alamar Blue VR (10%, Cedarlane Corp., Burlington, ON, Canada) for 4 hours, and the fluorescence emission intensity was measured (Aex 560 nm, Aem 590 nm) using a microplate fluorescence reader (BioTek Instruments Inc., Synergy 4, USA). To estimate cell viability, the fluorescence intensity of each sample was compared to those of positive and negative controls (cells exposed to culture medium and to 10% v/v dimethyl sulfoxide solution (DMSO) respectively).

### Results and Discussion

The pH measurements confirmed that all hydrogels presented almost neutral pH, excepted for the samples prepared with 0.1-0.2 M SHC and SHC0075:BGP that presented pH >7.5 (Table 2). As detailed below, the type and concentration of gelling agents added to chitosan solution strongly influenced the gelation kinetics, mechanical resistance and porosity of the hydrogels. All formulations led to hydrogels with pH close to physiological values. The pH of the filtrates was ≦7 for the CH:PB, CH:BGP and CH:PB:BGP samples, 7.4 for the CH:SHC samples, and between 7 and 7.4 for the CH:PB:SHC and CH:BGP:SHC samples. It was very difficult to recover liquid from the CH:SH0005 and CH:SH00075 hydrogels, which had almost no free water.

### Rheological Properties

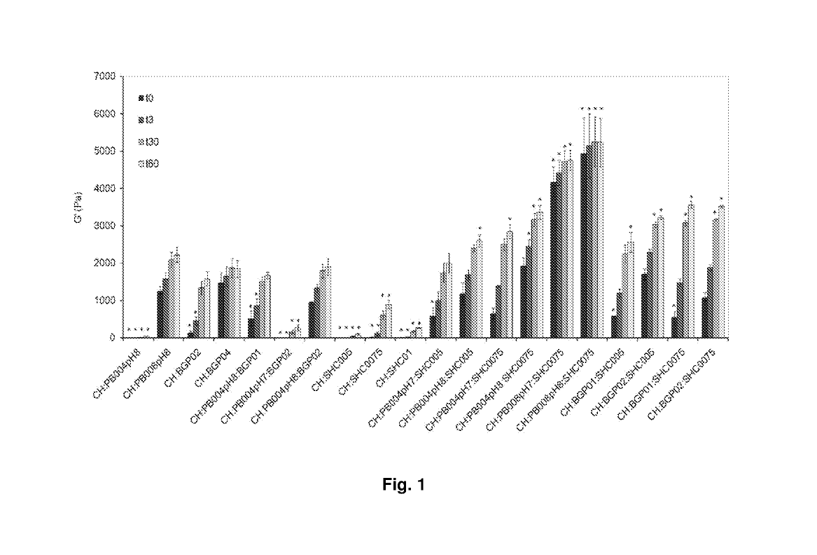

For concision purposes, only selected representative results of gelation kinetics at 37° C. are presented in FIG. 1. They are expressed by the storage modulus (G)′ values at t=0, 3, 30 and 60 min. Tests were also carried out at 22° C. and at 5-65° C. (rate of 1° C./min) to study the influence of temperature on gelation process (FIG. 2).

### Effect of Gelation Agent Combination on Rheological Behavior

CH Gelation with PB:

The storage modulus of CH-PB hydrogel increased with the increase of PB concentration and pH, reaching about 2200 Pa after 1 hour at 37° C. with CH:PB008pH8, while no gelation was observed with CH:PB004pH7 and very weak hydrogels were obtained with CH:PB004pH8 or CH:PB008pH7 (FIG. 1). These variations may be explained by the increase of SPD concentration when the concentration or the pH of PB increases. SPD, a weak base with a pK_{b} of 6.8 is known to induce CH gelation. Higher concentration of PB led to an immediate gelation and the obtained hydrogel was fragile.

CH Gelation with BGP:

As expected, the gelation rate and final storage modulus increased when BGP concentration increased from 0.1 M to 0.4 M (FIG. 1). This e was already reported and attributed to an increase of CH chains interaction, leading to an enhancement of polymer rigidity. The resistance to shear of CH-GBP however remains limited. Indeed, even with 0.4 M BGP, the storage modulus of the hydrogel did not exceed 2000 Pa after 1-hour gelation. Further increasing the BGP concentration to 0.6 M led to immediate formation of a fragile and unflowable hydrogel.

CH Gelation with SHC:

With SHC (0.05-0.2 M), the highest G′ and G″ were obtained when an intermediate concentration (0.075 M) was used (FIG. 1). Even at this optimal concentration, the hydrogel exhibited very poor mechanical properties after 1 hour (G′=900±110 Pa). Unexpectedly, the G′ and G″ values with 0.1-0.2 M SHC were lower than those obtained with CH:SH00075. This may be due to an increase in CO₂, which slowed down the sol-gel transition (equation 1).

CH—NH₃⁺+HCO₃⁻→CH—NH₂+H₂O+CO₂ (1)

Indeed, using the weak SHC base as a gelling agent leads to CO₂ generation after mixing with the acidic solution of CH.

CH Gelation with BGP:PB Combination:

Combining BGP with PB did not significantly improve chitosan gelation compared with BGP (or PB) alone. Moreover it was shown to strongly depend on the composition and pH of the PB solution. The storage and loss modulus of hydrogels prepared with BGP:PB004pH7 were remarkably lower than those prepared with BGP alone or with BGP:PB004pH8 (FIG. 1). Such results may be due to the higher contribution of PB004pH8 than PB004pH7 to the CH gelation and to the lower BGP ionization in PB at pH 7 than in Milli-Q water or in PB at pH 8, according to the following equilibrium:

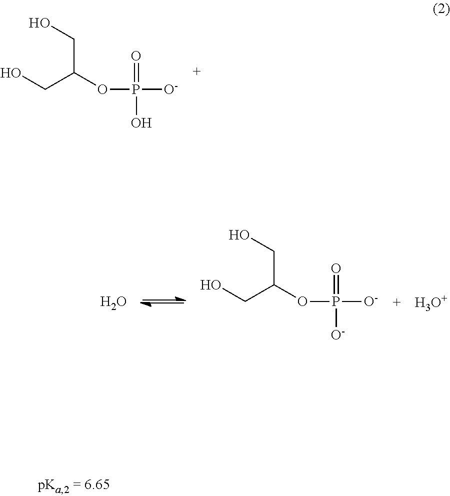

CH Gelation with SHC:PB Combination:

Combining SHC with PB had an important synergic effect on the gelation kinetic and mechanical properties of the hydrogels (FIG. 1). G′ values were significantly higher than those obtained with SHC or PB alone, and even higher than BGP04, even though the total salt concentration was lower. PB concentration of 0.08 M led to remarkably higher G′ than 0.04 M at each time point, (G′˜5000 Pa), but these values tended to progress less with time. Due to its lower pKₐ compared to BGP (6.35 vs. 6.65), SHC should present a higher percentage of ionization in PBpH7 than BGP. This may at least partly explain the much higher storage modulus values obtained with CH:SHC:PBpH7, compared with CH:BGP:PBpH7.

CH Gelation with SHC:BGP Combination:

Adding SHC to BGP also had a synergic effect on CH gelation (FIG. 1), where G′ reached 3000 Pa after 30 min. This synergic effect with SHC:BGP, as well as with SHC:PB, may possibly be explained by the smaller size of SHC molecules compared to BGP, a slower reaction of SHC than PB with the ammonium groups of CH and the decomposition of SHC in acidic medium. This effect may lead to stronger polymer network, as will be confirmed later during compression tests.

### Thermosensitive Properties of CH Gels

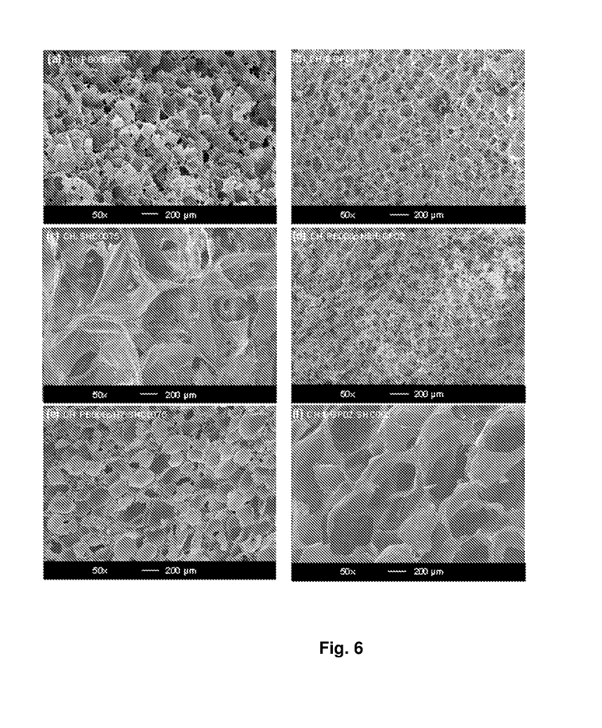

The influence of temperature on the rheological properties of the hydrogels was studied and some examples are shown in FIG. 6. The formation of all hydrogels was delayed at 22° C. compared with 37° C., and G′ were considerably lower (FIG. 1, FIG. 2 a-d). This confirms that all hydrogels are thermosensitive (thermogels). The gelation kinetics was however less affected by temperature variation when the concentration of the gelling agent increased (e.g., BGP02 vs. BGP04).

Upon heating from 5 to 65° C., the temperature at which G′ rapidly increases, which gives an indication of a temperature of incipient gelation, and the slope of this increase were both influenced by the kind and concentration of gelling agents (FIG. 2 e-f).

G′ increased much more rapidly and extensively in gels prepared with SHC and its combination with PB and BGP (FIG. 2f), compared to BGP or PB alone (FIG. 2e). In gels prepared with SHC0075, G′ increased suddenly to reach about 14 kPa but this increase took place at a temperature >40° C. (FIG. 2f). With SHC005:PB004pH8 and SHC005:BGP01, G′ started to increase remarkably at temperature >25° C. In contrast, with PB and BGP alone, G′ values were still below 2.5 kPa at the end of the test. G′ increase in PB008 gels began slowly at 15° C. while for BGP, the “threshold” temperature depended on BGP concentration, decreasing from 35° C. for BGP02 to 25° C. for BGP04. Excepted for CH:SHC, G′ profiles showed more than one stage, which may be due to the presence of more than one basicity in the medium.

Thus, adding SHC to PB or BGP leads to gels which sol-gel transition did not occur at room temperature, while at higher temperature the mechanical resistance increased considerably. This behavior is very interesting for thermosensitive injectable hydrogels, since it allows easy injection at room temperature and rapid in situ gelation at body temperature.

### Compression Strength of Hydrogels

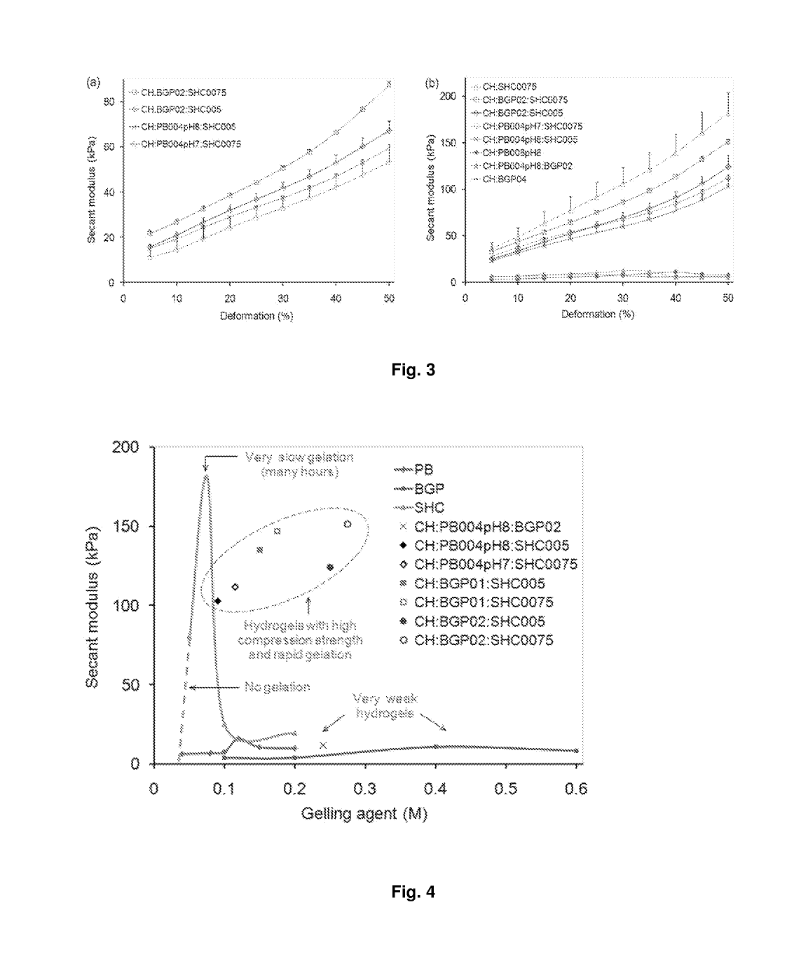

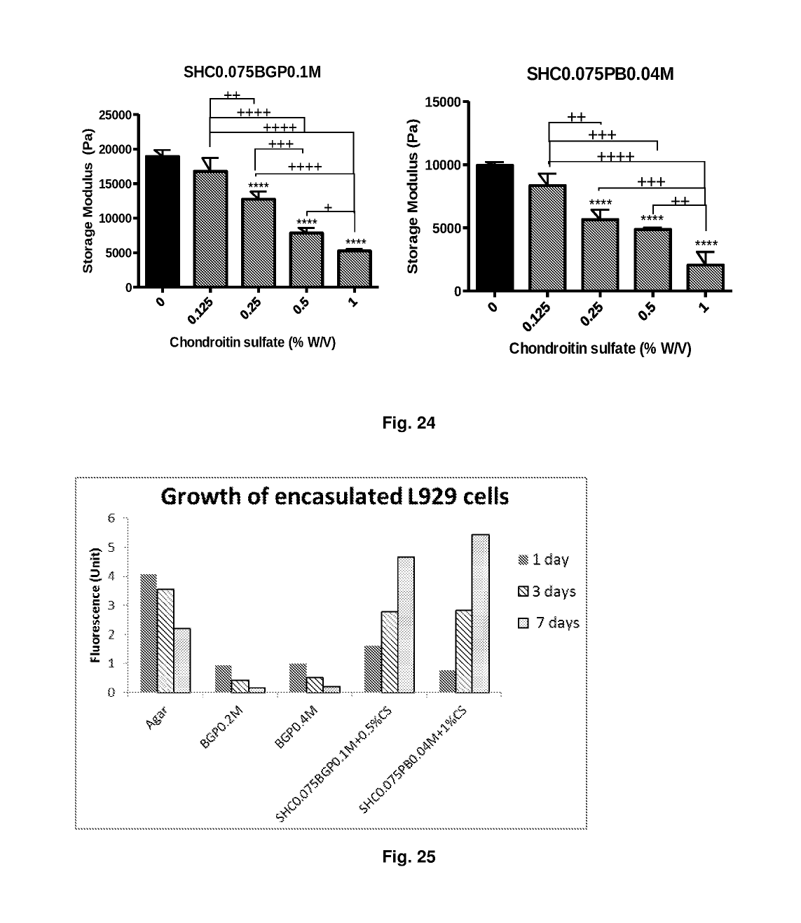

FIG. 3 presents the secant Young's modulus of various hydrogels obtained during progressive unconfined compression (up to 50%) after 1 h (FIG. 3a) and 24 h gelation (FIG. 3b). The modulus increased with the deformation, reflecting a typical non-linear behavior of a hydrogel. After 1 hour at 37° C., CH:SH00075 was still liquid and the hydrogels prepared with PB008pH8 and BGP04 were too weak to be handled and analyzed, while the hydrogels prepared with SHC:PB or SHC:BGP were already quite rigid and resistant. After 24 h at 37° C., the secant modulus was higher than that after 1 hour for each sample, indicating that the gelation process continued even after 1 hour. Unexpectedly, while hydrogels with PB008pH8 and BGP04 were still very weak, breaking up at <12 kPa, hydrogels prepared with SHC0075 presented a very high secant modulus, reaching ˜180 kPa at 5o % deformation. The high resistance of CH:SH00075 after 24 h gelation at 37° C. may be interesting, but the very slow gelation process limits the ability to use CH:SHC hydrogels in clinical applications. All samples with salt combinations containing SHC (PB:SHC or BGP:SHC) showed remarkably higher secant moduli (10- to 20-fold increase) than those obtained with BGP, PB or PB:BGP, and they recovered their initial cylindrical shapes after compression, while the latter hydrogels broke up during compression.

All samples with salts combination containing SHC (SHC:PB or SHC:BGP) showed remarkably higher secant moduli (10 to 20 fold increase after 24 h gelation at 37° C.) than those obtained with BGP04, PB008pH8 or BGP02:PB004pH8 and they kept their initial cylindrical shape after compression, while the latter hydrogels broke up during compression.

These data are summarized in FIG. 4 which presents the secant modulus as a function of concentration for each type of gelation agent or their combination. Altogether, rheological and compression studies show the large advantage of mixing SHC with other weak bases to reach rapid gelation at body temperature and strong CH hydrogels. Combining SHC with SPD only may also lead to a similar effect but this combination was not studied within the scope of this paper. The very low mechanical properties of BGP:PB, even after 24 h gelation, confirm that the addition of SHC is a key factor to increase the resistance of the hydrogels.

The gelation rate and the strength of the hydrogel may depend on the pK_{b} (HPO₄²⁻, 6.8; BGP²⁻, 7.35 and HCO₃, 7.65), the concentration, the charges and the size of the weak bases. Moreover, the pH and temperature of both CH and the gelling agent, before and after mixing, can influence the gelation. In addition, the heat may change the pKₐ or the ionization degree of the compounds, thus affecting the gelation process. In deep study would be necessary to understand the role of each factor and the mechanism of gelation. However, we can hypothesize that with BGP, the relatively big size of the molecule prevents the formation of strong network via multiple interchain bonds. The relatively fast reaction of PB with CH, due to the relatively lower pK_{b} of SPD, may create a non-homogenous distribution of the interactions within the polymer, thus affecting the creation and the properties of the polymer network. In contrast, the slow gelation with SHC0075 may permit a homogeneous neutralization of ammonium groups of protonated CH and then better physical junctions and interchain entanglements. Moreover, the decomposition of SHC after mixing with the acidic CH solution, contrarily to PB and BGP, may allow the chitosan chains to get closer, thus increasing their interaction.

### FTIR Analyses

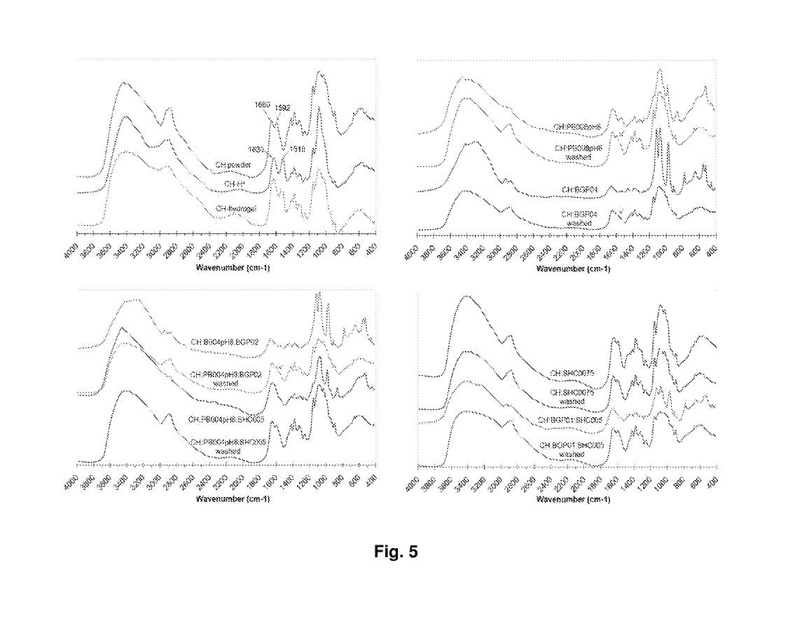

FTIR analyses were carried out on some CH and CH hydrogels samples with the aim to follow the modification on CH amino groups and the presence of salts in the hydrogels (FIG. 5). The spectrum of CH powder shows the characteristic absorption bands of this polymer as per, mainly O—H and N—H stretching vibrations at 3500-3200 cm⁻¹, C—H stretching at 2920-2878 cm⁻¹, amide I (carbonyl, C═O) stretching at 1660 cm⁻¹, deformation of primary amino group (NH₂) at 1592 cm⁻¹ overlapping that of lower intensity of amide II at 1556 cm⁻¹ (N—H), C—N stretching of C—NH₂ at 1422 cm⁻¹, O—H deformation of CH₂—OH at 1377 cm⁻¹ and O—H deformation of CH—OH at 1322 cm⁻¹ overlapping with CH₃ deformation and CH₂ wagging, asymmetric stretching of C—O—C bridge at 1151 cm⁻¹, C—O stretching of CH—OH at 1080 cm⁻¹, C—O stretching of CH₂—OH at 1032 cm⁻¹, CH₃ rocking at 993 cm⁻¹, and C—O—C stretching between the rings at 898 cm⁻¹ (FIG. 5 a).

The completely protonated CH (CH—H⁺) showed shifting to lower wavenumber values of amide I (1630 cm⁻¹) and amino groups (1518 cm⁻¹) (FIG. 5 upper left). The shifting and the intensity of the amino groups' band of protonated CH used to prepare hydrogels (CH-hydrogel) were relatively lower than those of completely protonated CH, indicating an incomplete protonation of CH. This partial protonation offers three advantages. First, easier interaction between CH chains in short distance may occur after adding weak base due to lower repulsion of protonated amino groups. Second, the presence of some unprotonated amino groups may be useful to increase polymer-polymer and water-polymer interactions. Third, the use of a lower quantity of acid to dissolve CH, requires a lower concentration of a weak base to neutralize the CH, thus favoring better biocompatibility for the hydrogel. Kim et al. (2010) proposed the dialysis of the acidic chitosan solution prior to adding BGP, thereby reducing the amount of BGP needed and improving biocompatibility. In the present study, using incompletely protonated CH may avoid the necessity of this long step.

The spectra of the unwashed dried hydrogels showed the presence of the typical bands of each gelling agent used in the preparation of the hydrogels as per (FIG. 5 upper right and lower left and right). SPM and SPD showed multiple intense bands ascribed to phosphate groups between 1400 and 800 cm⁻¹ while BGP showed a broad band at 3600-2800 cm⁻¹ corresponding to hydroxyl and alkyl groups, and intense bands at 1160-920 cm⁻¹ ascribed to phosphate groups. However, the bands corresponding to SHC at 1700-1600 cm⁻¹ and 1400-1300 cm⁻¹ had negligible intensity, maybe due to the decomposition of SHC after mixing with the acidic CH solution. After washing, the salts seemed to be removed completely from hydrogels, since the spectra did not show any band attributed to phosphate or carbonate. While the hydrogels with SHC, SHC:BGP and SHC:PB showed similar spectra to that of CH powder, the spectra with PB, BGP and PB:BGP showed modification at 1650-1500 cm⁻¹. This modification is likely due to incomplete deprotonation of CH. It seems that using SHC favors a complete neutralization of the CH, while using PB and/or BGP, even at higher concentrations, may hinder some —NH₂⁺ groups within the chain network.

### Morphology of Freeze-Dried Hydrogels

The morphology of the freeze-dried chitosan hydrogel was strongly affected by the kind and concentration of salts used (FIG. 6). SEM analyses showed that CH:BGP04 and samples containing PB presented a homogeneous porosity with interconnected pores. The pore size decreased when combining BGP with PB. In contrast, hydrogels prepared with SHC presented larger pores (300-400 μm), which may be explained by the formation of CO₂ bubbles during gelation. Depending on the targeted application, a smaller or larger porosity may be more favorable, since porosity influences cell invasion, tissue regeneration or drug release.

### Cytotoxicity of CH Hydrogels

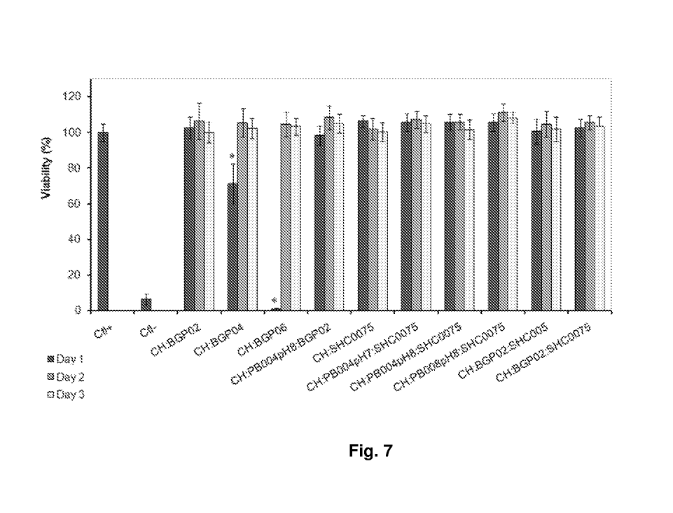

The incubation of L929 cells with the hydrogel extracts showed cytotoxic effect at BGP concentrations of 0.4 M and higher (FIG. 7), in agreement with a previous work on bone marrow-derived mesenchymal stem cells. This is believed to be due to a rapid release of BGP from the hydrogel during immersion in the culture medium, leading to hypertonicity of the media. Extracts from the subsequent incubations (48 hours, 72 hours) showed no toxic effect.

No cytotoxicity was noticed with any of the other hydrogels tested. This may be due to lower cytotoxicity of one gelling agent compared with the others but more probably to the fact that the combination of SHC with BGP or PB allows a reduction of the total salt concentration in the hydrogel. Indeed, previous results with SHC showed no damage of SHC 0.1M on endothelial-derived cells (EA.hy 926) while a higher concentration (2.38 M) had a strong cytoxic effect on NIH-3T3 fibroblast cells. These results suggest that the combination of SHC with BGP or PB, which allows a reduction of the total salt concentration in the hydrogel, improves the cytocompatibility of CH hydrogel.

The gelation rate and the strength of the hydrogel may depend on the pK_{b} (HPO₄²⁻, 6.8; BGP²⁻, 7.35 and HCO₃, 7.65), the concentration, the charges and the size of the weak bases. Moreover, the pH and temperature of both CH and the gelling agent, before and after mixing, can influence the gelation. In addition, the heat may change the pKₐ or the degree of ionization of the compounds, thus affecting the gelation process. We can hypothesize that with BGP, the relatively large size of the molecule prevents the formation of a strong network via multiple interchain bonds. The relatively fast reaction of PB with CH, due to the relatively lower pK_{b} of SPD, may affect the formation and properties of the polymer network, possibly by generating a non-homogenous distribution of the interactions, as observed with strong bases. In contrast, the slow gelation with SHC0075 may permit a homogeneous neutralization of ammonium groups of protonated CH, and thus better physical junctions and interchain entanglements. Interestingly, FTIR data suggest that the presence of SHC favors a complete neutralization of the CH. Moreover, the decomposition of SHC after mixing with the acidic CH solution, contrarily to PB and BGP, may keep the chitosan chains closer, permitting easier interaction. The formation of bubbles with SHC may not only influence the gelation kinetics, but entrapped bubbles could also play a role in the mechanical strength of the hydrogel. The notion of mixing SHC with BGP or PB to optimize or improve the gelation rate and the strength of hydrogel may be generalized to other weak bases or even to other polymers.

Altogether, these new injectable chitosan hydrogels provide very interesting features allowing clinical applications. First, combining SHC with PB or BGP leads to injectable gels at room temperature, while at higher temperatures, they gel rapidly and their mechanical resistance increases considerably. This thermosensitive behavior is very interesting for injectable hydrogels: it allows easy mixing with other products such as drugs or cells, and injection through small diameter catheters at room temperature, while rapid in situ gelation at body temperature prevents migration outside the targeted site. While CH-BGP also present thermosensitive properties, our results suggest that BGP concentrations required for rapid gelation (0.4 M and above) lead to hydrogels which are less stable at room temperature than the new formulations.

Another key issue is reaching high mechanical properties to withstand stresses and adequately function in vivo. This is true, for example, for the embolization of blood vessels, where the gel must be resistant to blood flow. Little data exists regarding the mechanical properties required to efficiently occlude blood vessels, but our previous work using in vitro embolization bench testing and animal experiments on other hydrogels suggests that good gel cohesion and a rapid increase of G′ to or above 1 kPa are minimum prerequisites for achieving these. Higher values are required for gels submitted to load-bearing applications such as intervertebral disk or cartilage. Hydrogels with enhanced mechanical properties are also important for pharmaceutical applications, especially for drug release, and for tissue engineering.

Finally, another advantage of the new thermogels is their improved cytocompatibility, as compared to CH-BGP gels, as shown by indirect cytotoxicity tests. Indeed, chitosans created with a concentration of 0.2 M BGP or less did not exhibit any cytotoxic effects, but gelled very slowly, which prevents their use for many applications. Extracts from chitosan prepared with sufficiently high BGP concentration to achieve rapid gelation (0.4 M BGP or higher) showed significant cell mortality, in accordance with previous studies. In stark contrast, none of the extracts from the new formulations showed any cytotoxic effect. This may be due to the lower cytotoxicity of the new gelling agents compared with BGP, but more probably to the fact that the combination of SHC with BGP or PB allows a reduction of the total salt concentration in the hydrogel, and thus avoids hyperosmolality, which can lead to cell damage and death. SHC is biocompatible at low concentrations, since it is an important part of the blood chemical buffer system. Previous results with SHC showed no damage of SHC 0.1 M in endothelial cells, but showed a strong cytotoxic effect at high concentrations (2.4 M).

### Conclusions

In this example, we drastically increased the Young modulus and mechanical strength of chitosan hydrogels without modifying chitosan, using ionic or covalent crosslinking or increasing the ions concentration. It should be noted that in come embodiments, the Chitosan could however be modified. A very interesting synergistic effect was observed when combining SHC with BGP or PB, reaching hydrogels with rapid gelation and drastically enhanced mechanical strength in shear and compression strength, despite even lowered salt concentration compared with CH:BGP hydrogels. These mechanical properties are key issues for injectable gels, which require easy injection but no significant migration and strong resistance to possible in vivo stresses. This is particularly true for the embolization of blood vessels, where the gel must be resistant to blood flow. Hydrogels with enhanced mechanical properties are also important for pharmaceutical applications, especially for drug release, and for tissue engineering. The idea of mixing SHC with BGP or PB to optimize or improve the gelation rate and the strength of hydrogel may be generalized to other weak bases or even to other polymers.

The hydrogels tested presented almost neutral pH, close to physiological pH. SHC is an economic product and is biocompatible at low concentrations, since it is an important part of blood chemical buffer system. Finally, compared to the commonly used CH:BGP hydrogels, for which a compromise must be found between gelation time and cytoxicity, CH:SHC:PB and CH:SHC:BGP hydrogels enable to optimize the gelation time and mechanical properties, while decreasing the cytotoxicity and keeping a porous homogenous structure which enables cell invasion. Overall, these thermosensitive hydrogels pave the way for new minimally-invasive treatments. More generally, the use of two weak bases as gellation agent shows promises in the manufacture of gels having various properties.

### Example 2—Optimized Formulations for Cell Therapy

Injectable hydrogels are increasingly used in biomedical applications since they provide an excellent platform for less invasive treatment and/or more local delivery of cells, drugs and/or other bioactive products. In particular, they look particularly interesting for cell therapy, a young and emerging sector which promises to deeply change the medical practices in the near future. Endogenous cells involved in the process of tissue regeneration of a damaged organ can be extracted, ex vivo expanded and re-implanted to increase the number of competent cells available at the injury site. Unfortunately, the efficacy of cell therapy is presently limited by the low number of functional cells due to early cell death and low retention at the targeted site after administration. Injectable scaffolds can ensure appropriate cell localization, retention, survival and protection from mechanical stresses.

One challenge is to develop smart biocompatible scaffolds that are liquid at room temperature to allow injection through a needle or a catheter but solidify in vivo to localize grafted cells near the targeted tissue and withstand in vivo stresses. The biomaterial should then degrade over time. This invention enables to fulfill these needs.

An example of thermoresponsive material is chitosan (CH) gelified in the presence of a basic salt such as beta-glycerophophate (BGP). Developed by Chenite et al. these hydrogels have been used to provide a platform to the host organism for in situ cell invasion and new bone tissue formation. Unfortunately, chitosan-BGP present slow gelation and poor mechanical properties. Gelation can be accelerated by increasing the BGP concentration, but above a certain limit the gel then becomes cytotoxic and incompatible for cell entrapment [2] due to its high ionic strength. This strongly limits the potential of this gel as a cell vehicle for in vivo administration, despite his numerous other advantages.

In this invention we propose new formulations which can simultaneously improve chitosan thermogels mechanical properties and reduce the total concentration of salts using the combination of sodium hydrogen carbonate (SHC) at precise concentration, with another weak base such as phosphate buffer (PB) or BGP. In this example, we further optimized and evaluated these hydrogels with respect to their use as injectable scaffolds for cell therapy, namely their osmolality, pH, gelation kinetic, thermosensitive properties, injectability and final mechanical properties. We then evaluated the potential of these hydrogels for cell therapies: a) L929 mouse fibroblasts, commonly used to evaluate the biocompatibility of the hydrogels according to ISO10993 standards; b) human mesenchymal stem cells (MSC) that can be used for cell therapy (using their paracrine effect or differentiation ability) and for tissue engineering. Results obtain with the new formulations were compared to chitosan/BGP thermogels.

### Materials

Shrimp shell chitosan (Kitomer, PSN 326-501, Premium Quality, Mw 250 kDa, DDA 94%) was purchased from Marinard Biotech (Rivière-au-Renard, QC, Canada). β-Glycerol phosphate disodium salt pentahydrate C3H7Na2O6P.5H2O (BGP), sodium phosphate monobasic NaH2PO4 (SPM) and sodium phosphate dibasic Na2HPO4 (SPD) were obtained from Sigma-Aldrich (Oakville, ON, Canada). Sodium hydrogen carbonate NaHCO3 (hereafter SHC) was purchased from MP Biomedicals (Solon, Ohio, USA). The other chemicals were of reagent grade, and were used without further purification.

### Preparation of CH Hydrogels

Chitosan solution: Chitosan (CH) was first purified following the method described in detail by Assaad et al.12 Chitosan powder was then solubilized in HCl (0.1 M) at 3.33% (w/v) overnight with a magnetic stirrer. The solution was sterilized by autoclaving (20 min, 121° C.) and stored at 4° C.

Gelling agent (GA) solutions: Three different GAs were used in this study, namely β-glycerophosphate (BGP), sodium hydrogen carbonate (NaHCO3, hereafter SHC) and phosphate buffer (PB), at pH=8, prepared with a mixture of sodium phosphate dibasic (Na2HPO4, SPD) and sodium phosphate monobasic (NaH2PO4, SPM) at a ratio of 0.932/0.068) in Milli-Q water. SHC was combined with either PB or BGP to obtain final concentrations described in Table 1. BGP at 0.02 and 0.04 M served as reference, because it was used to create CH-BGP hydrogels in previously published work.12 Gelling agents were sterilized by filtration through 0.2 μm filters and stored at 4° C.

Preparation of hydrogels for physico-chemical characterization: Chitosan hydrogels were prepared by mixing CH solution with one of the GA solutions at a volume ratio of 3:2 respectively. The two solutions were introduced in separate syringes, which were joined by a Luer lock connector. The contents of the syringe were pushed from side to side for 15 repeats immediately prior to use.

Preparation of hydrogels for cell culture: For cell encapsulation, hydrogels of similar composition were prepared, but in two consecutive steps: CH was first mixed with 2× concentrated GA solution (at a volume ratio of 3:1). The pre-formed hydrogel (already at physiological pH but still liquid) was then poured in one of the syringes and mixed with the content of a third syringe containing the cell suspension (5M cells/mL in complete medium) at a volume ratio of 4:1. The hydrogel containing cells (0.5 mL) was deposited in 48 well culture plates and left to gelify for 5 min. at 37° C. before adding 1 ml cell culture medium (see cell culture section) on top of the gel and putting it back in the incubator (37 ₉₂²³⁸C, 5% CO2). The cell culture medium was changed twice per week.

All formulations are summarized in Table 3. Final CH concentration in the hydrogel was kept constant at 2% (w/v). Throughout the balance of the paper, each hydrogel will be identified by its final molar concentration in GA. For instance, BGP0.1:SHC0.075 corresponds to a gel containing 2% (w/v) of CH, 0.1 M BGP and 0.075M SHC.

### Measurement of pH and Osmolality

After 24 hours of gelation at 37° C., each hydrogel was pressed and filtered in order to recover entrapped solution. The pH of the extracted liquids was measured at room temperature using a pH meter (UltraBasic pH-meter, Denver Instrument). The impact of GA concentration of gels prepared with culture medium (as detailed in Section 2.2.4) on the final osmolality of hydrogels was measured using an osmometer (Advanced® Micro Osmometer, Model 3300, Advanced Instruments Inc.).

### Rheological Tests

Rheological properties were investigated using an Anton Paar instrument (Physica MCR 301, Germany) with coaxial cylinder geometry (CC10/T200). The storage modulus (G′), loss modulus (G″) and initial complex viscosity were measured in the linear viscoelastic range, at a constant shear stress (1 Pa) and constant frequency (1 Hz), immediately following the preparation of each hydrogel. To investigate the thermoresponsive properties of the hydrogels, rheological parameters were recorded during temperature ramps between 4° C. and 65° C. (1° C./min). The gelation kinetic was also followed during isotherm at 37° C. and at 22° C. for 1 hour.

### Injectability

Injectability of thermogels through small diameter catheters (length=150 cm and inner diameter=0.53 mm, Boston scientific, Fas Tracker™-18 MX Microcatheter) was first verified manually, and then by measuring the maximal force required to extrude hydrogels using the ElectroForce® 3200 instrument (Bose Corporation, USA) equipped with a 220 N load cell. Immediately after mixing CH and GA, the hydrogel was placed in a 1 mL syringe connected to the catheter previously filled with NaCl 0.9% at 37° C. The catheter was filled with gel and left for 5 minutes at 37° C. The force required to extrude the gel was then monitored, and the maximum force recorded for the various formulations was compared. The morphology of the extruded material (continuous thread, segments, droplets, etc.) was also observed.

### Mechanical Properties

Unconfined compression tests were performed using a Bose ElectroForce® 3200 instrument equipped with a 22 N load cell. Two (2) mL of hydrogel was poured into cylindrical containers (14 mm diameter) and incubated at 37° C. for 24 hours for gelation. Compression was applied at a constant rate of 0.05 mm/s (100% deformation/min.?) until reaching 50% deformation. The secant Young's moduli were calculated as the slope of a line connecting the point of zero strain to a point at a specified deformation. Tensile tests were also performed on rectangular samples measuring 26×20×2.5 mm.

### Morphology Analysis by Scanning Electron Microscopy (SEM)

The morphology of the hydrogels was investigated by scanning electron microscopy (SEM, Hitachi S-3600). Two (2) mL of hydrogels were prepared in 48-well plates and incubated at 37° C. for 24 hours for gelation. The resulting hydrogels were frozen at −20° C. overnight, and freeze-dried under vacuum for 24 hours. The dried hydrogel were carefully cut in the thickness using a surgical blade (no 22, Surgeon®), deposited on double-coated carbon conductive tape and coated with a gold layer using an Emitech K550X sputter coater (Quorum Technologies Ltd).

### Cell Culture

L929 mouse fibroblast cells (ATCC, Manassas, Va., USA) were routinely cultured in normal Dulbecco's Modified Eagle's Medium (DMEM, Gibco BRL, Invitrogen, Grand Island, N.Y., USA) supplemented with 10% fetal calf serum (FCS; Medicor, Montreal, QC, Canada) and 1% glutamine (PS, Gibco BRL, Invitrogen) until 90% confluence before being passaged. L929 were used at passages 10 to 15. Human MSCs (hMSCs, StemCell Technology, Canada) were expanded in supplemented serum-free MSC NutriStem XF medium (Biological industry, Israel) and used at passages 4 to 6.

### Live-Dead Staining

After 24 hours of encapsulation in the various hydrogels, cell viability and repartition of live and dead cells in hydrogels were evaluated by Live-Dead assay with a viability/cytotoxicity kit (FluoProbes®, Interchim). Hydrogels were washed once with DMEM without FCS and immersed (45 min, 37° C.) in the presence of 2 μM ethidium homodimer-3 (necrotic marker measuring nucleus membrane integrity) and 1 μM calcein AM (viability marker measuring intracellular esterase activity) to stain dead cells in red and live cells in green. After staining, hydrogels were washed with DMEM without FCS and observed in fluorescent microscopy (Leica DM IRB) at 5× magnification.

### Metabolic Activity

The metabolic activity of encapsulated cells was evaluated by Alamar Blue (AB) VR (10% v/v Cedarlane Corp., Burlington, ON, Canada) assays at 1, 3 and 7 days. To optimize the access of AB to entrapped cells, it was mixed with the hydrogel by gently pipetting up and down with a P1000. After 3 hours of incubation at 37° C., 100 μl of supernatant was transferred to a 96-well plate, and fluorescence emission intensity was quantified using a microplate fluorescence reader (Aex 560 nm, Aem 590 nm, BioTek Instruments Inc., Synergy 4, USA).

### Statistics

All experiments were repeated at least in triplicate. Results are expressed as mean±SD. Statistical comparison of the data was performed using GraphPad Prism software with a two-way ANOVA and post-Bonferroni's test for comparison of more than two groups. A value of P<0.05 was considered significant.

### Results

Hydrogels with Physiological pH and Osmolality for Cell Encapsulation:

For cell therapy and tissue engineering applications, we optimized and preselected the hydrogel formulations. To reach gels with physiological pH, SHC-PB gels were prepared with PB at pH8 using mixture of sodium phosphate dibasic (Na2HPO4, SPD) and sodium phosphate monobasic (NaH2PO4, SPM) at a ratio of 0.932/0.068) in Milli-Q water. Hydrogel osmolality was measured for various concentrations of GA, with the aim of targeting GA concentrations and combinations that presented osmolality values close to physiological conditions (250-400 mOsmol/L for mammalian cell culture).

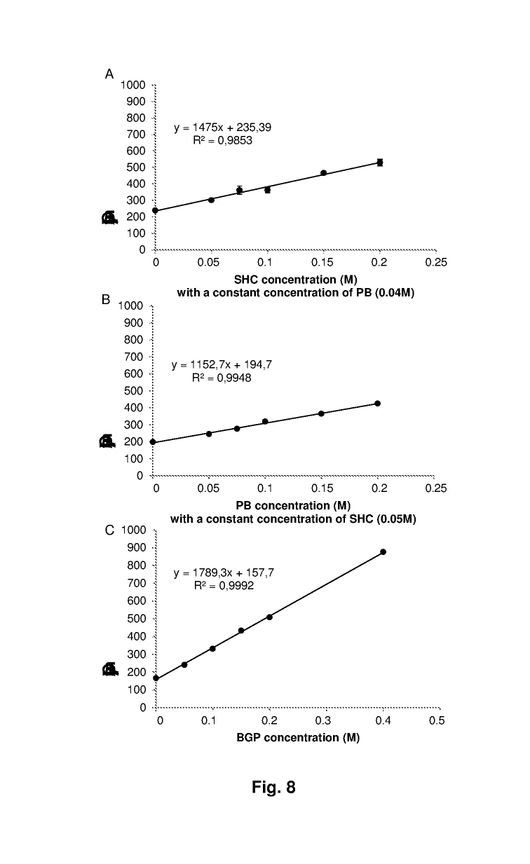

As expected, the osmolality increased linearly with the concentration of gelation agent, but with different slopes (FIG. 8, osmolality in mOsm/L on the vertical axis). BGP rapidly increases the osmolality (slope Panel C=1789) and hydrogels are already hypertonic at 0.2 M BGP (516 mOsmol/L). The hyperosmolality of hydrogels prepared with 0.4 M BGP (873 mOsmol/L) may explain the extent of cell death obtained with these gels in previous publications. In contrast, PB and SHC had a lower impact on the final ionic strength (SHC: slope _{Panel A}=1475, PB: slope _{Panel B}=1153)

A theoretical equation of the osmolality as a function of the hydrogel formulation was derived from these curves: Osmolality (mOsmol/L)=158+1475 [SHC]+1153 [PB]+1789 [BGP], where [SHC], [PB] and [BGP] are the molar concentrations (M) of SHC, PB and BGP respectively, and 158 is the osmolality due to the addition of culture medium in the gel at a 1:4 volume ratio. These data enabled to determine the value of SHC, PB and BGP where hydrogels remain relatively iso- or even hypo-osmotic New formulations prepared with SHC-PB and SHC-BGP0.1 M were iso- or hypo-osmotic. An hypo-osmotic gel is not a problem since NaCl can thus be added to the gelling agent to reach 300-320 mOsm/L without affecting the rheological behavior of thermogels (data not shown). It can even been interesting since it permits to add other components like bioactive molecules in the formulation of hydrogel without exceed isotonic values.

Based on these osmolality data and results presented in Example 1, formulations using SHC at 0.05 and 0.075 M, combined with PB at 0.004 and 0.008M or with BGP0.1M were kept for the cell encapsulation study (see Table 3). All were isotonic and at physiological pH. The conventional chitosan-BGP hydrogels (with BGP concentrations of 0.2 M and 0.4 M) that are usually described in the literature were kept as controls for comparison.

Another Concern about BGP

The possible second limitation of BGP concerns its osteogenic differentiation potential on stem cells. BGP is reported to be a differentiation agent of stem cells toward osteogenic lineage [Bruedigam, C., et al.,]. It may thus be interesting to avoid its use for other purpose, to prevent the formation of ectopic tissue in vivo when implanted in other organs. SHC-PB hydrogels offers this opportunity.

### Method of Preparation for Cell Encapsulation

Results from example 1 were obtained by mixing chitosan solution with the gelling agent without cells. Here, we first demonstrated the feasibility of physically encapsulating cells in hydrogels. For these preliminary tests, we used mouse fibroblasts (L929 cell line). The first method tested was to pour the gel in a tube containing cell pellet and suspend them in the pre-formed gel. With this method, the results were not optimal because of the formation of bubbles when pipetting, the impossibility to aspirate it again in the syringe and finally, the cells distribution was not homogenous in the hydrogel. As expected, suspending cells directly in the gelling agent led to important cell death due to non physiological pH and high salt concentrations. Another method was therefore developed to mix the cells within the hydrogels. This methods implies 2 steps and 3 syringes.

The first step consists in mixing chitosan with concentrated gelling agent with a Luor Lock connector. Once homogenous, this solution is still liquid at room temperature and can be mixed with the solution containing the cells using a third syringe using a similar luer lock system. For example the gelling agent was prepared at a concentration which is 5 fold the final value expected in the gel, and mixed with chitosan solution at a volume ratio chitosan:gelling agent of 3:1. Then the cell suspension (in culture medium or physiological solution) is mixed to the gel solution at a ratio hydrogel:cell suspension of 4:1).

After 24 hours of encapsulation in hydrogels, cells were stained with Live/Dead and observed in fluorescence microscopy. Most cells were alive after encapsulation process and they were distributed homogeneously throughout the hydrogel (data not shown) Moreover, we evaluated the impact on cell viability when a high pressure is applied on the syringes. No evident difference in terms of cell viability was observed compared to a gentle mixing. This suggests that cell viability is not operator dependent.

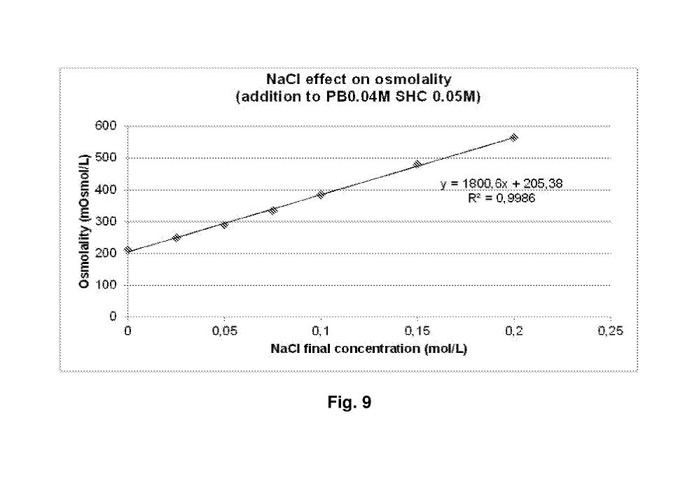

With this encapsulation procedure, cells can be incorporated in physiological serum, buffers for cell culture or cell culture medium. This solution affects the final osmolality of the hydrogels. Therefore, osmolality of the different formulations prepared with a ratio of pre-formed hydrogel:cell culture medium of 0.8:0.2 (reproducing the cell encapsulation method detailed above) was measured and is presented in Table 3. We can see an increase in osmolality for all hydrogels and still a large advantage of the new formulation compared with BGP 0.2 and 0.4M. Moreover, the addition of NaCl enables to adjust the osmolality at physiological values (FIG. 9).

### Rheological and Mechanical Properties

### Thermosensitivity and Gelation Kinetic

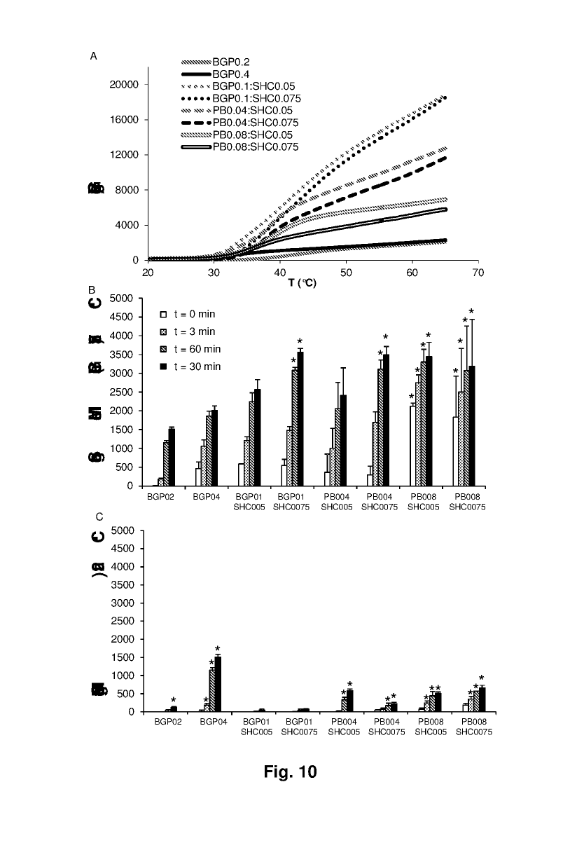

FIG. 10A presents the evolution of the storage modulus (G′, in Pa) of hydrogels during temperature ramps from 4° C. to 65° C. A sudden increase of G′ is observed for all gels between 30° C. and 40° C., demonstrating thermosensitive properties and suggesting a sol-gel transition temperature close to body temperature (37° C.). Interestingly, the G′ increase is much more pronounced with BGP0.1:SHC (0.05 M and 0.075 M) hydrogels compared to chitosan-BGP hydrogels, while PB:SHC formulations presents intermediate values.

FIG. 10B presents the values of G′, again in Pa, at 0, 3, 30 and 60 minutes during isotherms at 37° C., and The gelation time at 37° C. (time where G′=G″ according to the approach from Winter and Chambon14 was less than 15 s for all hydrogels except for BGP0.2 (35 s) (data not shown). Hydrogels prepared with 0.2 M BGP presented a slower gelation kinetic with a relatively low storage modulus even after 60 minutes (1587±186 Pa). By increasing the concentration of BGP to 0.4 M, it was possible to accelerate gelation but G′ after 1 hour was only improved by 17% (1850±227 Pa). The new formulations exhibited various gelation kinetics, but all of them presented increased storage modulus at 1 hour compared with BGP0.4 (p<0.05 for BGP0.1:SHC0.075, PB0.04:SHC0.075, PB0.08:SHC0.05, PB0.08:SHC0.075). The highest final G′ value was obtained with BGP0.1:SHC0.075 (3563 Pa), despite its low initial value. PB0.04:SHC0.075 showed higher initial G′, which may be interesting for applications where gel migration must be avoided. Interestingly, we noted that a minute increase of SHC concentration (from 0.05 M to 0.75 M) can have a strong impact on final shear properties. In contrast, PB concentration mainly influenced the gelation kinetic, where hydrogels prepared with PB0.08 leading to much higher initial G′ values than their PB0.04 counterparts.

FIG. 10C presents the variation of G′, in Pa, during isotherms at 22° C. For all formulations except BGP0.4, G′ values increased very slowly and remained below 1000 Pa within 1 hour, thus demonstrating their relative stability at 22° C. This allows to easily and homogenously mix the cell suspension in hydrogels and is a first indication of their injectability.

### Injectability of the Hydrogels Through Small Lumen Catheter

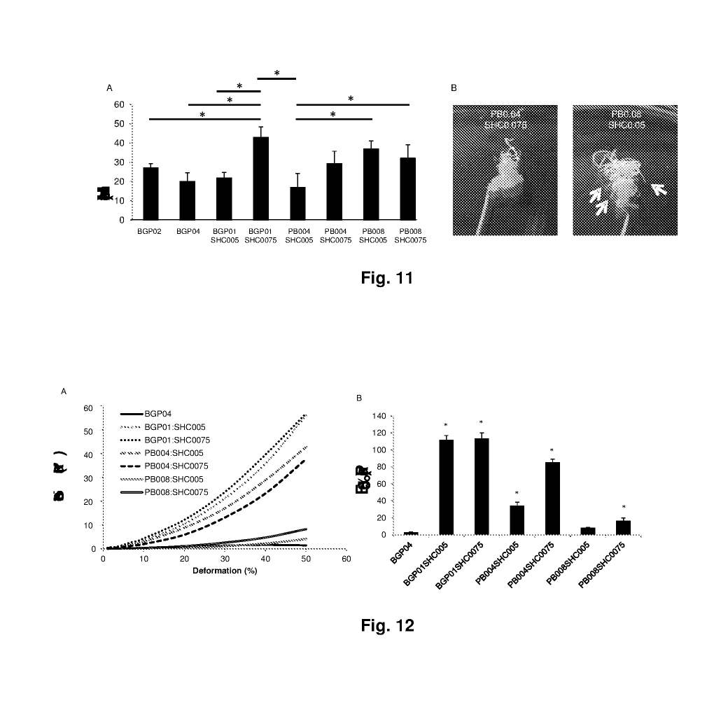

The Injectability of thermogels through small diameter catheters (length=150 cm and inner diameter=0.53 mm, Boston scientific, Fas Tracker™-18 MX Microcatheter) was first verified manually, and then by measuring the maximal force required to extrude hydrogels using the ElectroForce® 3200 instrument (Bose Corporation, USA) equipped with a 220 N load cell. Immediately after mixing CH and GA, the hydrogel was placed in a 1 mL syringe connected to the catheter previously filled with NaCl 0.9% at 37° C. The catheter was filled with gel and left for 5 minutes at 37° C. The force required to extrude the gel was then monitored, and the maximum force recorded for the various formulations was compared. The morphology of the extruded material (continuous thread, segments, droplets, etc.) was also observed. Results are reported in FIG. 11A in Newtons. All formulations were manually injectable through this catheter geometry, and the maximal force required varied between 17 and 43 N, with no direct correlation with rheological properties of the hydrogels. Extruded hydrogels were very cohesive, and formed a linear and continuous shape after injection in a saline bath at 37° C. (as represented in FIG. 11B for PB0.04:SHC0.075), with the exception of PB008 formulations, which led to more discontinuous segments after extrusion (see FIG. 11B, arrows)

### Mechanical Properties

Unconfined compression tests showed a drastic increase in the mechanical properties of the new formulations compared to conventional chitosan-BGP hydrogel, as shown in FIG. 4. FIG. 12A presents the typical stress-deformation curves of hydrogels after 24 hours of gelation at 37° C., while FIG. 12B summarizes the secant Young's moduli obtained at 50% deformation for all formulations, both vertical axes being in kPa. BGP0.2 was still very soft after 24 hours of gelation, and was too weak to be handled for compressive tests. Hydrogels prepared with BGP0.4 broke at approximately 30% deformation, and their average secant modulus was only of 3.0±0.5 kPa. In contrast, BGP01:SHC and PB004:SHC hydrogels were cohesive and easy to handle, even after only 1 hour of gelation. They sustained 50% deformation, and their secant moduli and strength in compression were drastically enhanced compared to other formulations. The highest values were obtained with BGP0.1:SHC0.075 (113.5±6.4 kPa), which secant modulus at 50% was up to 37 times higher than BGP0.4.

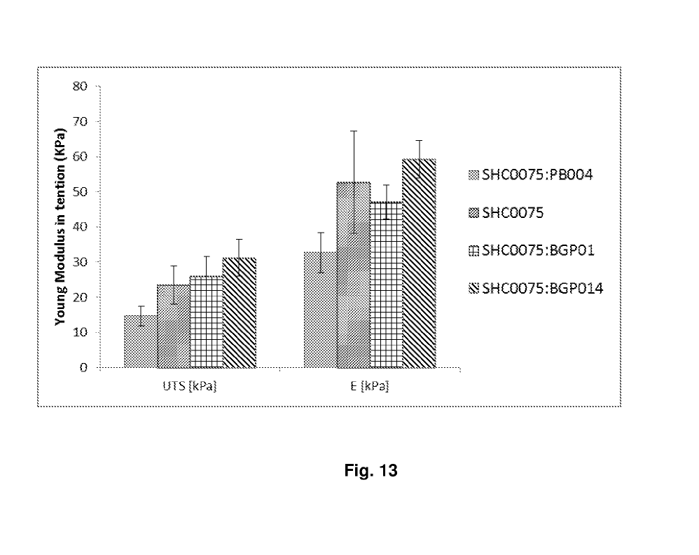

The new hydrogels also showed good resistance in tension, with a relatively linear behavior (data not shown). Young's moduli and ultimate tensile strength (UTS) reached 47 kPa and 25 kPa respectively for BGP0.1:SHC0.075. (FIG. 13) Mechanical properties decreased when increasing PB concentration in SHC-PB gels.

### Morphology of Lyophilized Hydrogels (SEM)

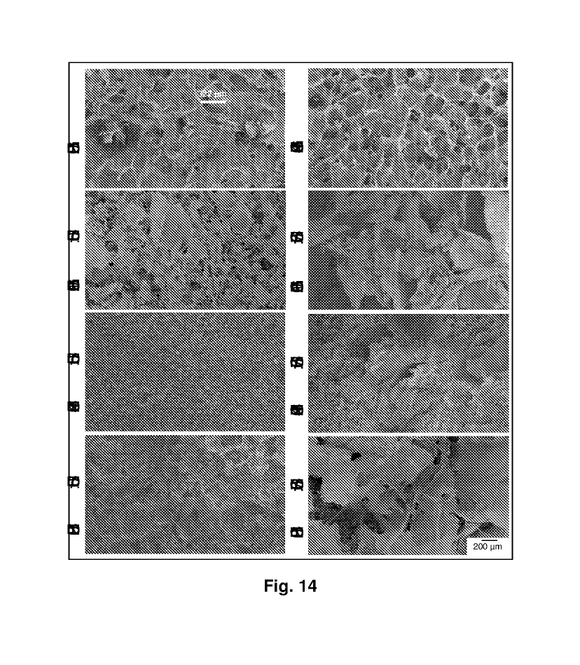

SEM images of hydrogel cross-sections showed that the type and concentration of GA can impact the final ultrastructure of the hydrogel (FIG. 14). BGP-based hydrogels presented a homogenous and interconnected porosity with a pore size of approximately 300 μm according to the micrographs. Formulations prepared with a mix of BGP and SHC resulted in structures presenting larger pores with a less homogenous size distribution. Hydrogels obtained with PB and SHC presented a smaller but homogenous porosity. Although the freeze-dried porosity does not represent the hydrated porosity, because the freeze-drying process creates artifacts, these data suggest that changing the type and concentration of GA allow for the creation of different morphologies that can have an impact on cell proliferation and escape from hydrogels.

### In Vitro Cell Cytocompatibility

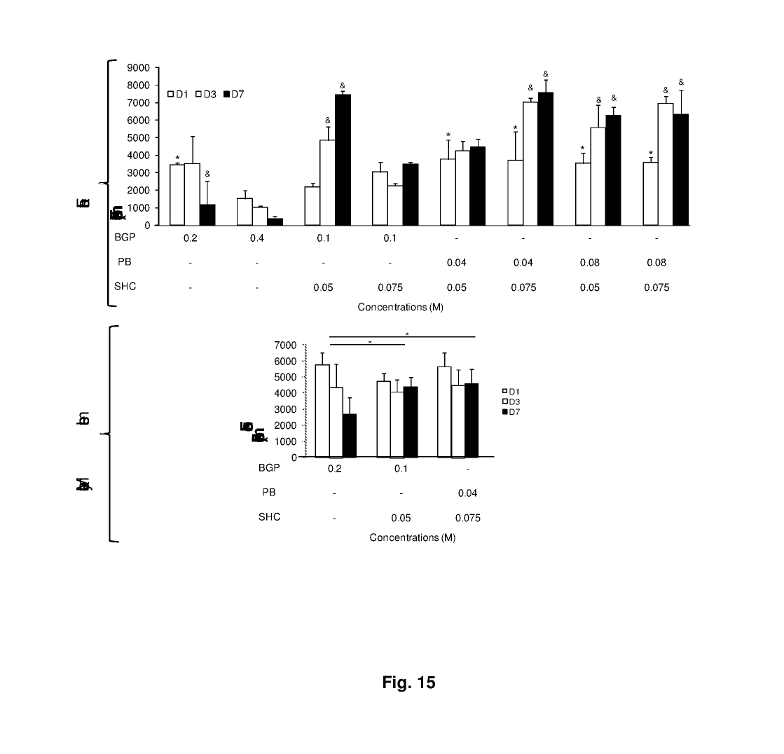

L929 were encapsulated in each formulation of hydrogels. Cell metabolic activity was followed over 7 days (FIG. 15A, as measured by fluorescence emission intensity at 530 nm), and live/dead staining was performed after 24 hours of culture. As expected, encapsulation in BGP0.4 led to significant cell death, as shown by numerous red cells in live/dead at day 1 and a decrease in cell metabolic activity over time. The initial viability was better with BGP0.2, but the cell number also decreased over time. The metabolic activity of entrapped fibroblasts was significantly improved in the new hydrogels, with marked improvement at day 7 for all formulations. The variation of measured metabolic activity over time tends to differ among the formulations. A clear increase in metabolic activity was observed for BGP0.01:SHC0.05, PB0.04:SHC0.075, PB0.08:SHC0.05 and PB0.08:SHC0.075 (p<0.05 compared to day 1), which suggests that they were able to sustain significant cell proliferation.

### Viability and Proliferation of Mesenchymal Stem Cells in Hydrogels

The two best formulations obtained previously (BGP0.1:SHC0.05 or PB0.04:SHC0.075) were tested for their ability to encapsulate human MSCs, in comparison with BGP0.2. Once again live/dead staining was performed at 24 hours, and metabolic activity was tested by AB for 7 days (FIG. 15B as measured by fluorescence emission intensity at 530 nm). The initial metabolic activity and Live/Dead staining showed excellent cell survival and no difference between the formulations tested after 24 hours encapsulation. However, after 7 days of culture, significant cell mortality was observed with the BGP02M control, whereas cell metabolic activity remained constant in the new formulations.

### In Vivo Implantation

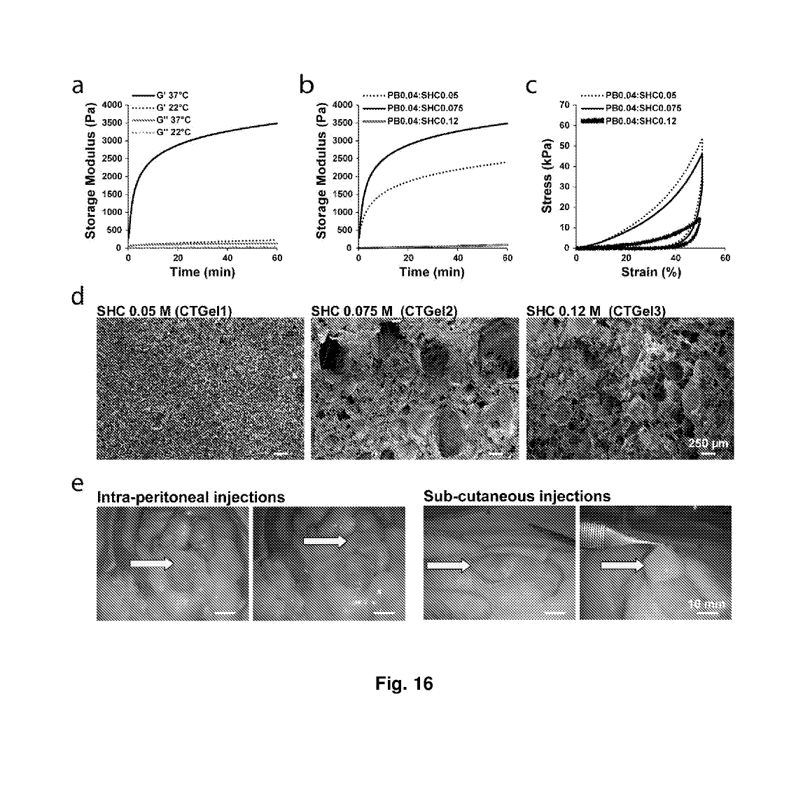

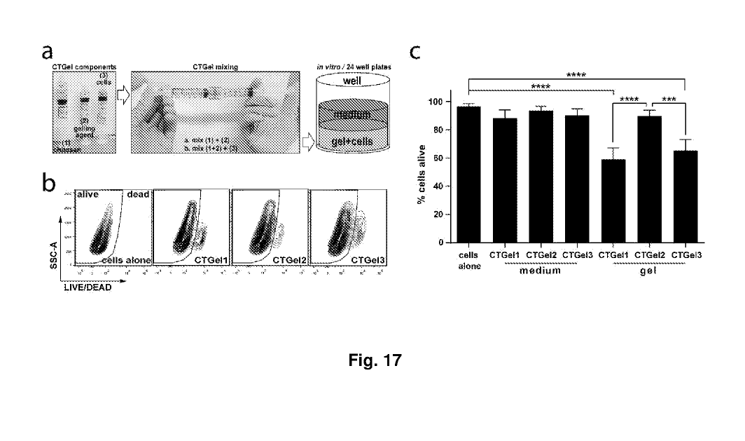

We evaluated the possibility to implant hydrogels by intra-peritoneal and sub-cutaneous injections in rats. We injected 2 ml of hydrogels (chitosan2%/PB0.04MSHC0.075M) in each case and we autopsied animals after 15 minutes (FIG. 16e).

Hydrogels were injected in abdominal cavity or under the skin and were retrieved 15 minutes after. In the abdominal cavity, the hydrogel was found near the bladder. For sub-cutaneous injection hydrogels stayed under the skin, at the injection point. For all injection sites, hydrogels were into a cohesive form.

### Discussion:

Here are some possible advantages of the present CH hydrogels for cell therapy:

The sensitivity to temperature is very useful for in situ cell delivery (homogenous mixing with cells before viscosity increases too much; low viscosity for injectability; rapidly forms a solid cast). The high mechanical resistance is a very important factor to withstand in vivo stresses and keep the form of the scaffold (load bearing applications, embolization).

The gel is highly cytocompatible thanks to the decrease of salt concentration, the maacroporosity, the absence of cross-linking agent and the high ratio of water as all hydrogels. Cells survive and grow for prolonged period of time. Scaffolds of different porosity can be created, depending on the needs. The optimal porosity and range of osmolality may vary depending on the type of cells, as illustrated by the differences observed for cTL in SHC005PB004 and SHC0075PB004.

Chitosan is biodegradable and playing on chitosan degree of deacetylation (DDA) or molecular weight (MW) can influence its biodegradation rate.

Scaffolds containing not only cells but also bioactive products such as drugs or growth factors can be created.

### REFERENCES FOR THIS EXAMPLE

1. Roche, E. T., et al., Comparison of biomaterial delivery vehicles for improving acute retention of stem cells in the infarcted heart. Biomaterials, 2014. 35(25): p. 6850-8.
2. Wang, L. and J. P. Stegemann, Thermogelling chitosan and collagen composite hydrogels initiated with beta-glycerophosphate for bone tissue engineering. Biomaterials, 2010. 31(14): p. 3976-85.
3. Cho, J., et al., Physical gelation of chitosan in the presence of beta-glycerophosphate: the effect of temperature. Biomacromolecules, 2005. 6(6): p. 3267-75.
4. Cheng, Y. H., et al., Thermosensitive chitosan-gelatin-glycerol phosphate hydrogels as a cell carrier for nucleus pulposus regeneration: an in vitro study. Tissue Eng Part A, 2010. 16(2): p. 695-703.
6. Engler, A. J., et al., Matrix elasticity directs stem cell lineage specification. Cell, 2006. 126(4): p. 677-89.

### Example 4—Application for T Cell Therapy (Immunotherapy)

Adoptive cell therapy (ACT) has emerged as a promising anti-cancer therapeutic strategy, and its successes rely on the perfused CD8⁺ T lymphocyte's ability to gain access to, and persist within the tumor microenvironment where it must maintain its cytotoxic phenotype to carry out its functions. The major current drawbacks of this therapeutic application is the need to cultivate large numbers of tumor-derived cells; limiting patient eligibility, and the loss of many of the adopted cells to sites of non-specific inflammation. An objective of the present example is to show that chitosan-based hydrogels can act as an injectable cell-delivery vehicle, in order enhance this branch of cancer-immunotherapy by reducing both the numbers of cells required and their non-specific loss by locally delivering these as a concentrate to sites of cancer. For this, Three thermogel formulations, prepared with SHC0075M and PB, with acceptable physicochemical properties, such as physiological pH and osmolality, macroporosity, and gelation rates were compared. The CTGel2 formulation outperformed the others by providing an environment suitable for the encapsulation of viable CD8⁺ T lymphocytes, supporting their proliferation and gradual release. In addition, the encapsulated T cell phenotypes were influenced by surrounding conditions and by tumor cells, while maintaining their capacity to kill tumor cells. This strongly suggests that cells encapsulated in this formulation retain their anti-cancer functions, and that this locally injectable hydrogel may be further developed to serve as a tertiary lymphoid structure-like mimic towards the complementation of current immunotherapies.

### Introduction:

Systemic adoptive T lymphocyte transfer is an emerging cancer immunotherapy showing tremendous promise in clinical studies, and its successes relies on the ability of antigen-experienced and activated cytotoxic T lymphocytes (cTL) abilities to access and persist in the tumor microenvironment where they can have an anti-cancer effect (Restifo, Dudley et al. 2012). One main pitfall of the procedure is the need to expand enormous numbers of patient derived tumor infiltrating lymphocytes (TIL) ahead of the intended therapy, as many of those perfused into patients will be lost to non-cancerous sites of inflammation. Another is the toxicities observed from the intravenously administrated high-dose bolus IL-2 after cell transfer (Hershkovitz, Schachter et al. 2010). As both of these pitfalls of current adoptive cell transfer (ACT) therapies are associated to systemic application, we chose to develop a new method of local implantation of 3 dimensional (3D) TIL cultures into the tumor microenvironment akin to post-operative radiation therapy applied to cavities formed from tumor resection, with the intent to create injectable artificial mimics of tertiary lymphoid structures (TLS) associated to positive patient prognosis.

Chitosan, a deacetylated derivative of chitin, is a copolymer consisting of two repeating units (N-acetyl-2-amino-2-d-glucopyranose and 2-amino-2-deoxy-d-glucopyranose) that are linked by a β-(1→4)-glycosidic bond. Due to its being natural, biocompatible, biodegradable, and its having low immunogenicity, chitosan has been examined for its used towards a wide range of biomedical applications (Rhee, Park et al. 2014). When combined with a weak base, chitosan can form a thermosentive hydrogel. Chenite et al. in 2000 were the first to describe thermosensitive chitosan hydrogels by employing β-glycerophosphate (BGP) as gelling agent (Chenite, Chaput et al. 2000). With this combination, they obtained thermogels that gelify at 37° C. and at physiological pH. This encouraging result permitted it to be considered for biomedical applications, but its mechanical properties and biocompatibility remained to be improved. We have recently overcome these limitations by replacing BGP with a combination of phosphate buffer (PB) and sodium hydrogen carbonate (SHC), as detailed elsewhere in the present document. We were able to fine-tune gelation kinetics, mechanical properties, and morphology of these hydrogels using various PB and SHC concentrations that target desired specifications. With this approach, we are able to prepare hydrogels that gelify faster allowing the local administration of a gel that would gelify faster than it can be dispersed after injection. The hydrogels also have higher mechanical properties, are macroporous and are able to support cell growth relative to classical chitosan/BGP thermogels.

Though proliferating T cells have never been cultured in biogels for this purpose, we hypothesized that the implantation of 3D T cell cultures (or TLS-like structures) composed of hydrogels and concentrated cTL into the tumor microenvironment would provide a means for the delivery of a continuous feed of these cells towards the reduction of tumor burden. Therefore, our aim was to fine-tune a chitosan-based hydrogel formulation that is biocompatible, injectable and which has enough cohesiveness to produce a solid 3-dimensional (3D) structure after gelation. The structure would have to allow proliferation and release of cTL whose activation state can be influenced by the surrounding conditions, so that chemoattractants from the tumor microenvironment may accelerate the proliferation, release, and the immunogenicity of the encapsulated cells. We here expected to minimise the gelling time and to improve its mechanical properties to avoid the dispersion of the hydrogel after injection. We have chosen one concentration of PB which permits it to be injectable and three different concentrations of SHC for the generation of gels having different porosities which can influence cell proliferation and release.

Three formulations: CTGel1 (PB0.04M/SHC0.05M), CTGel2 (PB0.04M/SHC0.075M) and CTGel3 (PB0.04M/SHC0.12M) have been investigated with the aim to create thermogelling chitosan having different morphologies. Their rheological properties and mechanical strength were evaluated by rheometry and unconfined compression tests (10% of strain), respectively, and their morphologies and porosity were observed by scanning electron microscopy (SEM). The potential for thermogel biocompatibility and cell encapsulation was assessed using rheometry, and measures of pH and osmolality. Their capacities for their uses as therapeutics in animals was assessed by measuring gelation times, and by injecting the gel into rats for visualization of gel containment to sites of injection. Thermogel cytocompatibility was determined using time course experiments of encapsulated primary T cells. Gel- and supernatant-derived cells were followed over time, where cell numbers, viability, phenotype and activation status were recorded by microscopy and flow cytometry. We have determined that Gel2 is superior to the others tested for its abilities to provide an environment suitable for the growth of a 3D T lymphocyte cell culture that divides into visible colonies, and which favors the proliferation and escape of CD8+ T cells, of T cell clones, and of tumor infiltrating lymphocytes (TIL).

### Experimental Methods:

### Hydrogels Preparation:

Chitosan (Marinard Biotech, Mw 250 kDa, DDA 94%) was purified using sodium dodecyl sulfate as described in example 1. Chitosan solution was then obtained by solubilizing purified chitosan in HCl (0.1 M) at 3.33% (w/v) overnight at room temperature. The resulting chitosan solution was sterilized by autoclaving (20 min, 121° C.) and was stored at 4° C. until further use. Gelling agent solutions were prepared by mixing SHC and PB at pH 8 (prepared by mixing sodium phosphate dibasic (Na2HPO4, SPD) and sodium phosphate monobasic (NaH2PO4, SPM) at a ratio of 0.932:0.068) in Milli-Q water (EMD Millipore). These were prepared to obtain hydrogels with a final PB:SHC concentration of 0.04 M:0.05 M (CTGel1), 0.04 M:0.075 M (CTGel2) and 0.04 M:0.12 M (CTGel3). Gelling agents were sterilized by filtration through 0.2 μm filters and were stored at 4° C. until further use.

All CTGels were prepared to reach a final chitosan concentration of 2% (v/w). Chitosan solution (3.33%, v/w) was loaded into a syringe and the gelling agent solution was loaded into another (volume ratio of 3:2). For mixing, the two syringes were joined by a luer lock connector (Qosina, USA), and syringe contents were pushed from side to side for 15 repeats.

### Morphology, pH and Osmolality of Injectable Hydrogels:

After 24 hours of incubation at 37° C., hydrogels were squeezed through a 0.2 μm filter. The pH of the recovered liquid was then determined using a pH-meter (UltraBasic pH-meter, Denver Instrument, USA) and the osmolality was determined using an Advanced™ Micro Osmometer 3300 (Advanced Instruments Inc., Norwood, USA). For SEM analysis, gels were frozen at −20° C., freeze-dried, cut into sections using a surgical scalpel, and deposited onto double-coated carbon conductive tape before being metalized with gold. Their morphologies were then analyzed using a Hitachi S-3600 SEM.

### Rheological Properties and Mechanical Strength:

The gelation kinetics of CTGels at physiological (37° C.) and room (22° C.) temperature were studied by following their rheological properties using a Physica MCR 301 (Anton Paar, Germany) equipped with coaxial cylinder geometry (CC10/T200). The evolution of the storage (G′) and loss (G″) moduli was determined in the linear viscoelastic range at a constant shear stress (1 Pa) and at a constant frequency (1 Hz) over the course of 60 min. The time at which G′=G″ represents the gelation time and changes in G′ indicate the progressive gelation and increase of the elastic properties of the gel over time. The Young's modulus and mechanical strength in compression after 24 h of gelation at 37° C. were determined using an ElectroForce 3200 test instrument (Bose Corporation, USA) with a 22 N load cell. Samples were prepared in 14 mm inner diameter cylinder molds. Unconfined axial compression of up to 50% strain was applied at a rate of 0.5 mm per min. As the hydrogels present non-linear elastic behavior, the secant modulus was calculated. Each experiment was performed in triplicate.

Injection of Thermogels into Rats:

The injectability and cohesion of injected hydrogel were tested in rats kept under general anesthesia (isoflurane 2.5%). All in vivo experiments were conducted according to the Canadian Council on Animal Care guidelines for care and use of laboratory animals, and under the supervision of our institutional animal care committee. Chitosan, gelling agent and cell culture medium were mixed at room temperature (RT) at the volume ratio of 3:1:1 to prepare CTGel2 (PB 0.04 M:SHC 0.075 M), and then 2 mL of the solution was immediately implanted either by subcutaneous or intraperitoneal injections using 1 inch-long 23G needles. Rats were euthanized 10 min later by intraventricular exsanguinations and thermogels were surgically removed for macroscopic observation

### Cell Cultures and Conditions:

The ability of T lymphocytes to grow within and escape from the hydrogel was first verified by expanding these from PBMCs derived from healthy donors, and then on T cell clones and TIL expanded from renal clear cell renal carcinoma (ccRCC) tumors. Prior to CTGel encapsulation, T cells were expanded from PBMCs in Iscove's modified Dulbecco's medium (IMDM) complete medium, composed of IMDM (Invitrogen) supplemented with 7.5% decomplemented human AB serum (Sigma), 2 mM L-glutamine, 100 U/mL penicillin, 100 g/mL streptomycin, 10 g/mL gentamicin (Wisent), 0.5 mg/mL anti-CD3 (OKT3, eBioscience) and 1800 U/mL IL-2 (PeproTech). For TIL expansion, freshly resected ccRCC kidney tumors were cut into small fragments and then further homogenized using the gentleMACS tissue dissociator (Miltenyi Biotec, USA). The resulting tumor fragments were cultured in IMDM complete medium supplemented with 1800 U/mL IL-2 for 15 days, where half of the media was replaced after the first five days, and every three days thereafter as previously described [5]. The anti-gp100 HLA-A2-restricted T cell clone (directed against a gp100-derived peptide 209-217 in complex with HLA-A*02, and specifically targeting cancer cell lines SK23-mel and 624-mel) (a kind gift of Mark Dudley; Surgery branch, NCI, NIH), was grown using a REP as previously described [30-32]. Briefly, irradiated (5,000 rads) feeder cells (2.5×107) and anti-gp100 T cells (5×105) were cultured in Aim-V, 7.5% AB medium; composed of Aim-V (Invitrogen) supplemented with 7.5% decomplemented human AB serum (Sigma), and 2 mM L-glutamine, 100 U/mL penicillin, 100 g/mL streptomycin, 10 g/mL gentamicin (Wisent), 0.5 mg/mL anti-CD3 (OKT3) and 300 U/mL IL-2. The REPs were supplemented with 300 U/mL IL-2 on day 2, and 20 mL of the medium was replaced at days 5 and 8 where cells were then maintained at 1×106 cells/mL and IL-2 was added every 3 days until experiments. Melanoma cell lines SK23-mel, 624-mel, and 586-mel (established at the NCI/NIH Surgery Branch), and the breast tumor cell line MDA231 (ATCC) were grown in RPMI 1640 medium supplemented with 10% heat-inactivated FBS, 2 mM L-glutamine, 100 U/mL penicillin-streptomycin, and 10 μg/mL gentamicin.

### Cell Encapsulation:

Under sterile conditions and under a laminar flow hood, CTGel-T cell encapsulation was performed in two successive steps using the following component ratios: 0.6 mL chitosan solution was first mixed with 0.2 mL of gelling agents (containing PB and SHC, at double concentration) using two syringes and a luer lock connector. After 15 repeated mixings, the contents were shifted entirely to one of the two syringes. The now empty syringe was replaced with a new one containing 0.2 mL cells in complete medium supplemented with 1800 U/mL IL-2 (8×106 cells/mL), and again, the two syringes were joined through a luer lock for 15 rounds of mixing. The CTGel-encapsulated cells (1 mL/well) were then deposited into 24-well plates and incubated at 37° C. with 5% atmospheric CO2 for 5 min before being topped with 1 mL of pre-warmed media and placed back into the incubator until further processing or analysis.

### Live/Dead Assay:

CTGels were collected at indicated times post cell-encapsulation. For imaging analysis, the gels were rinsed with IMDM and stained (45 min, 37° C.) using green-fluorescent calcein-AM to indicate intracellular esterase activity of living cells and red-fluorescent ethidium homodimer-1 binding to the DNA of dead cells, according to the manufacturer's instructions (LIVE/DEAD Viability/Cytotoxicity kit, Life Technologies). After staining, hydrogels were washed with IMDM and observed using fluorescent microscopy (Leica DM IRB) at a 5× magnification.

### Flow Cytometry:

Cells were collected from both the media and the gels at indicated times post cell-encapsulation. Cells in the media were first collected using aspiration with one additional rinsing with IMDM. Cells from the gel were collected by washing gels twice with IMDM, then homogenizing gels using three consecutive cycles of the h_tumor_01 program of the GentleMACS™ Dissociator (Miltenyi Biotec, USA), and passing the resulting suspension through a 0.45 μm filter fit onto a 50 mL falcon tubes (Fisher) with two additional PBS washes prior to cell pelleting by centrifugation (4° C., 10 min, 1500 rpm). Cells were counted and resuspended in PBS for transfer to 5 mL polystyrene round bottom FACS tubes (Falcon) where non-specific binding sites were blocked with human gamma globulin (Jackson ImmunoResearch) and dead cells were labelled for flow cytometry-mediated elimination using a LIVE/DEAD fixable Aqua Dead Cell Stain Kit (Life technologies) for 20 min at 4° C. Following a cold PBS wash and centrifugation (4° C., 5 min, 1500 rpm), cells were resuspended in cold FACS buffer (PBS containing 0.5% BSA and 0.1% NaN3) and were stained for 30 min at 4° C. with the following titrated monoclonal antibodies CD3-FITC, CD4-APC-H7, CD8-PerCP-Cy5.5, HLA-DR-AF700 (BD Biosciences), and CD25-BV605 (BioLegend). For intracellular marking, cells were washed once with FACS buffer (4° C., 5 min, 1500 rpm), and were then treated using the FoxP3/Transcription Factor Fixation/Permeabilization Concentrate and Diluent kit (eBiosciences) for 30 min. Cells were then washed with, and resuspended in 1× cold permeabilization buffer (eBiosciences) for staining with titrated monoclonal antibodies: Granzyme B-APC, Perforin-PE, TNFa-PE-Cy7 and AnnexinV-v450 (BD Biosciences) for 30 min. Cells were washed with, and resuspended in cold FACS buffer for flow cytometry analysis. In multi-parametric FACS analyses, compensation beads (BD Biosciences) stained in parallel to cells were used to compensate for fluorescence spill over. Flow cytometry data was acquired using an LSR Fortessa cell analyzer with DIVA software (BD Biosciences) and data was analyzed using FlowJo V10 software.

### CFSE Labelling:

For recognition assays, CFSE labelling was used to identify either gel-encapsulated TIL or the melanoma and breast cancer cell lines. Briefly, cells were labeled using (5-and-6)-carboxyfluorescein diacetate (CFSE) (Molecular probes) diluted in DMSO (5 mM). Cells were first washed twice in PBS to remove any residual serum from the growth media, and were resuspended (1×107 cells/mL) in RPMI. CFSE solution was added at 1:1000 (final concentration of 5 μM) for incubation for 15 min in a 37° C. water bath with regular mixing. To stop the labeling, FBS (10% of total volume) was added for 1 min at RT, and cells were washed with RPMI before being centrifuged (4° C., 10 min, 1500 rpm) and were then resuspended at required concentrations for assays.

### Recognition Assay:

Recognition/killing assays were performed using the anti-gp100 T cell clone or TIL and their respective cognate cancer cells and tissues, respectively, and using Boyden Chambers 24 well plate cell culture inserts/transwells having 3 μm pore size polyethylene terephthalate bottom membranes (BD Falcon). For assays using anti-gp100 T cells, the REP expanded T cells in Aim-V, 7.5% AB medium supplemented with 300 U/mL IL-2 were encapsulated at 8×106 cells/mL of CTGel2, and 0.25 mL of cell-gel mixture was deposited into the Boyden/transwell top inserts and allowed to solidify for 5 min at 37° C. During this time, CFSE-labeled target cancer cell lines were seeded in the bottom wells of 24 well plates. The transwell inserts containing the gel-encapsulated cells were placed within the wells, and were topped with an additional 0.25 mL of Aim-V, 7.5% AB medium supplemented with 300 U/mL IL-2. The assay was allowed to continue for a period of 5 days at 37° C. with 5% atmospheric CO2, and then all cells were collected from the medium in the 24 well plate below. The TIL recognition assay was performed in the same way with the following modifications: the TIL were CFSE-labeled and gel-encapsulated, IMDM complete medium supplemented with 1800 IU/mL IL-2 was used, and the tumor fragments from which the TIL were originally expanded were placed in the bottom of the 24 well plates. After 5 days, cells were collected from both the bottom media in the 24 well plate wells and the gel in the top insert, as described earlier using the GentleMACS method. Supernatants were used for the detection of IFN-γ secretion as evaluated by ELISA.

### Results and Discussion:

### Physiochemical and Mechanical Properties and Injectability of Novel Chitosan Thermogel Formulations:

Ultimately, we aimed to refine an injectable thermogel formulation to create one well suited for the viability and growth of a 3D T cell culture. CTGel formulations were modified according to our hypothesis that, by increasing SHC concentration, we would increase porosity and firmness of the gel. High porosity of gels is associated with cell escape and provides good intra-gel exchange with factors from surrounding conditions. It was previously shown that SHC forms pores in the chitosan thanks to CO₂ bubbles generation at acidic pH (the initial CH solution is at pH 6.2). Consequently, we developed CTgel1 (SHC0.05M), CTgel2 (SHC0.075M) and CTgel3 (SHC0.12M), where all three formulations contain the same percentage of Chitosan and concentration of PB, but with varying concentration of SHC.

Table 4 describes the three CTGel formulations and summarizes the physicochemical data. All gels were found to be at physiological pH and at near-physiological osmolality (from 308 to 411 mOsm/L relative to physiological values in the 280-350 mOsm/L range), which are essential requirements for cytocompatibility allowing cell encapsulation.

CTGel rheological properties observed at 37 and 22° C. confirmed that formulations led to thermosensitive hydrogels, as shown in FIG. 16a, which presents the evolution of the storage (G′) and loss (G″) modulus of the CTGel2 at both temperatures. At 22° C., G′ and G″ remained low for an extended period of time, making the gel easily injectable by extrusion through a needle or a catheter. More importantly, this feature enabled the homogeneous mixing of the cells and CTGel (already at physiological pH) prior to injection. Contrarily, a rapid increase of G′ was observed at 37° C. For CTGel1 and CTGel2, gelation occurred within the first minute at 37° C. (where G′>G″ at the beginning of the test) and these presented relatively similar gelation kinetics, with rapid increases of the storage modulus (2405 Pa and 3490 Pa after 1 hour, respectively) (FIG. 16b). This rapid gelation and increase in mechanical properties is particularly convenient to obtain a strong and cohesive structure following injection, and particularly one which will not diffuse into other in vivo compartments. By comparison, CTGel3 had a slow gelation kinetic with a delayed gelation time of 12 min, and a poor storage modulus after one hour of gelation at 37° C.

Unconfined compression tests after 24 h of gelation at 37° C. confirmed the high mechanical properties of the CTgels, with some advantage for the CTgel1 and CTgel2 relative to the CTGel3 formulation, as shown by the stress-strain curves (FIG. 16c) and by the values of the E50% secant modulus (Table 4). These values are much higher than conventional CH-BGP gels (with 0.4M of BGP) for which the average E50% was found to be bellow 5 kPa [26]. While the complete mechanism involved in the formation of these thermogels has not yet been completely resolved, our data show that SHC concentration plays a key role in their final mechanical properties.

SEM analysis of the ultrastructure of freeze-dried hydrogels showed differences in pores densities and size distributions (FIG. 16d). As expected, increasing SHC concentration led to increased macroporosity. While direct extrapolations based on pore size should be avoided since freeze-dried porosity does not represent the hydrated porosity, these data show that minute changes in SHC concentrations can influence the ultrastructure and will likely impact T cell proliferation and escape.

Since it appeared to have the best overall characteristics, CTGel2 was selected for in vivo testing in rats. Intraperitoneal and subcutaneous injections of CTGel2 confirmed that it formed an injectable scaffold able to quickly gelify at physiological temperature and form a solid 3D structure in vivo. Ten minutes following injections, rats were euthanazied and the gels were explanted, and these presented a continuous and cohesive structure having a solid appearance (FIG. 16e).

CTGel Formulations are Permissive to T Cell Viability and Growth:

To determine whether the CTGels were cytocompatible for T cell encapsulation, T lymphocytes expanded from normal donor PBMCs were CTGel-encapsulated as depicted in FIG. 17a. After five days, cells were collected from the media and gels for analysis using live/dead staining analyzed by flow cytometry. Results demonstrate that there was no significant difference in cell viability for the cells released from the three CTGel formulations relative to the control cells collected from the medium (˜90%) (FIG. 17b&c). On the contrary, significant differences were observed for the cell viability of encapsulated T cells as a function of CTGel formulation. The viability of cells encapsulated in CTGel2 (90.33%±3.79) was largely superior to those isolated from the other two formulations (CTGel1: 59.33%±8.08; CTGel3: 65.67%±7.77; p<0.001, FIG. 17c).

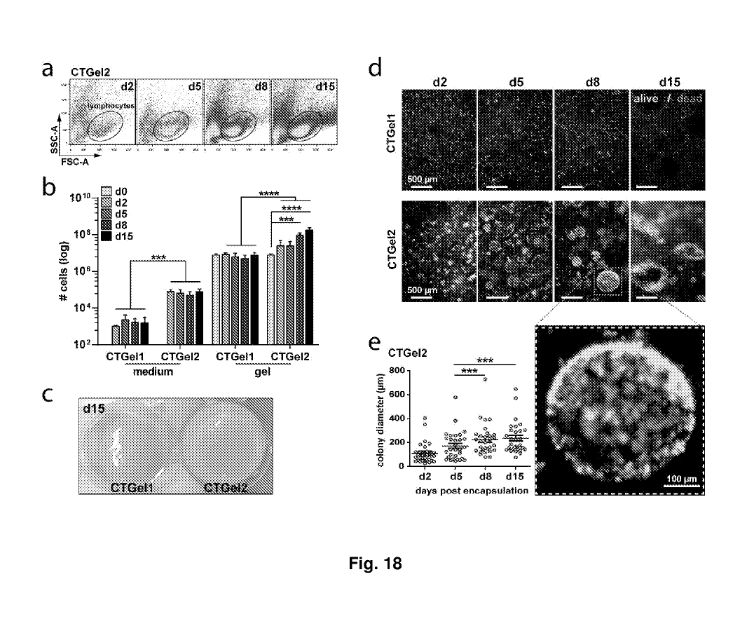

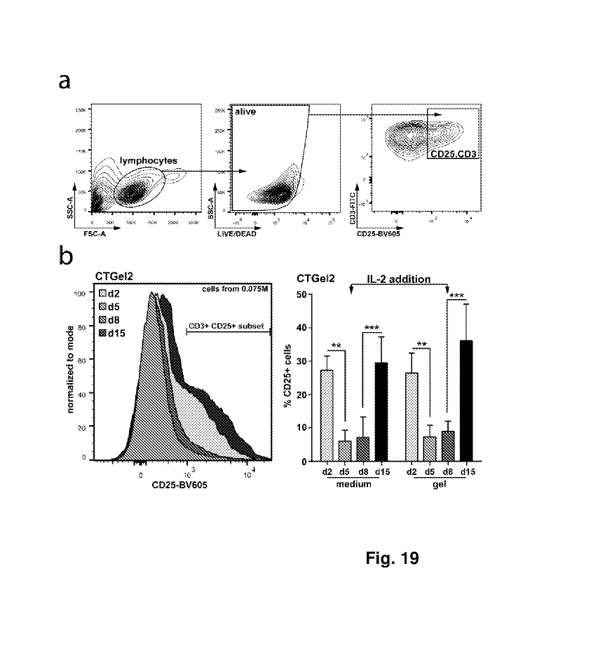

The growth of CTGel-encapsulated T cells was then followed over a 15-day time course, where cell numbers from the medium and from the gel for three different normal donors were determined at every three days post T cell-encapsulation using flow cytometry. FIG. 18a represents the gating on T lymphocyte morphology used to generate cell counts from the gel component of the experimental analysis to exclude other particles (i.e., CTGel remnants) also detected by the flow cytometer, but at different sizes) during the analysis of encapsulated cells. Results demonstrate that a stable number of cells was detected from the medium over this time course, with more cells escaping CTGel2 than CTGel1 overall (CTGel3 was not included in testing due to its low gelation rate) (FIG. 18b). Among the T cells isolated from the CTGels, we observed a similar steady-state in cell numbers over time in the CTGel1 formulation. In contrast, cell number increased in the CTGel2 formulation (i.e., from 25.7×106 at day 2 to 180.5×106 at day 15) (FIG. 18a&b). Macroscopic examination of the gel color over time was also a clear indicator of T cell growth, with a visually apparent progressive yellowing of CTGel2 over time, relative to the pink color (originally provided by the growth media) maintained by the CTGel1 (FIG. 18c).

These results were confirmed by direct visualization of CTGel-encapsulated T cells using live/dead microscopy. Strikingly, the CTGel2 formulation was clearly permissive to the growth of encapsulated T cells that increased in number over time, and colonies that increased in size over time, with diameters spanning as large as 375 μm by day 15 (FIG. 18d&e). With cells much denser at colony edges, these encapsulated colonies represent an intriguing aspect of the 3D culture, and are likely highly dependent on heightened gas diffusion and nutrients exchange throughout this gel formulation as a result of its increased porosity, as observed by SEM (FIG. 16d). Differences in cell viability between CTGels 1 and 2 could be explained by their difference in pore size. Pore size varying from 50 μm to 500 μm, as observed for CTGel2, are in accordance with the pre-established pore sizes (100-500 μm) that have been correlated with optimal cell seeding, proliferation and migration of cells, and with the diffusion of nutrients throughout gel scaffolds.

During these experiments, from microscopic examination of the gel using live/dead immunofluorescence, we also observed that the numbers of dead cells remained quite constant over time, and that many of these were localized to the bottom and sides of the gel where it would have been tight contact with the 24 well plates, and where we would expect that there would be the lowest amount of air exchange, or the longest possible time for temperature adjustments, and altogether indicating that the dead cells (represented by red throughout imaging), are likely a population of cells that dies during, or very early after cell encapsulation (FIG. 18d). To confirm that the numbers of dead cells were staying constant over time, we examined our flow cytometry data as we had in FIG. 17b-c. Analysis revealed that the amount of dead cells detected by flow cytometry remains constant over time, and importantly, that cell viability in CTGel2 remained above 85% over the 15-day time course experiment (data not shown). Altogether, these results indicate that of the three formulations tested, CTGel2 is best suited for encapsulated T cell viability, proliferation and escape from gels over time, and therefore the remainder of experiments presented herein are those using CTGel2.

CTGel-Encapsulated T Cell Phenotypes can be Influenced from Surrounding Conditions:

In line with our objective to develop a growing 3D T cell culture that can be injected into the tumor microenvironment and once there, release cells over time in response to its chemical cues and chemoattractants, we evaluated whether surrounding conditions could positively influence the activation state of the encapsulated T cells. We thus performed 15 day time course experiments where cells were encapsulated, and cells and media were again collected over time for analysis as before, but with the provision that the media was replaced with fresh, IL-2 containing medium at day 8 in an effort to demonstrate that IL-2 addition could boost the activation state of the encapsulated T cells, as demonstrated by increased CD25 expression. As CD25 is the high-affinity IL-2 receptor, and its expression on T lymphocytes is increased upon activation, it is routinely used in flow cytometry to identify activated T lymphocytes. FIG. 19a depicts the gating strategy applied for the analysis of the flow cytometry results, whereby (from left to right) cells are gated for morphology, then viability, and then for activated CD3+ T cells (i.e., CD3+CD25+). FIG. 19b&c reveals that at day 2, cells from CTGel2 were observed to still be highly activated from their growth in OKT3+IL-2 containing media prior to encapsulation (26.5%±6.0), and this activation state was diminished by day 5 (7.3%±3.5). However, a surge of reactivation of the cells was observed to occur following the addition of fresh IL-2 containing media to the culture condition at day 8, returning them to a high level of activation by day 15 (36.1%±11.1) (FIG. 19b&c). Altogether, these results demonstrate that the CTGel acts as a cellular support that permits the efficient exchange of cellular nutrients and chemical cues from the surrounding conditions to influence the activation state of the encapsulated T cells.

The CTGel Favors the Growth of CD8+ T Lymphocytes:

Cancer immunotherapy ACT protocols use CD8+ T lymphocytes expanded from resected patient tumors because these are the immune cells best recognized to have an anti-tumor effect and to positively impact patient prognosis. Therefore, we assessed the cellular phenotype of the encapsulated T cells to verify that it was not altered from growth in CTGels.

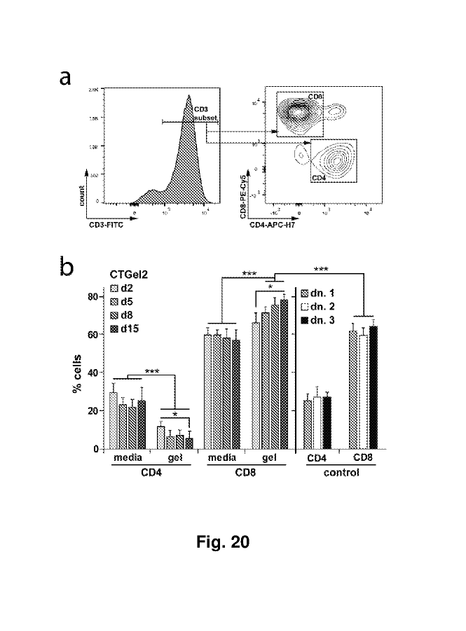

Fifteen-day time course experiments were performed to determine the phenotype of CTGel2 encapsulated T cells. FIG. 20a depicts a portion of the gating strategy used in the analysis of the flow cytometry data; where the full gating strategy was: morphology, singlets, alive, CD3+, and CD8+ or CD4+. Results show that after expansion in the CTGel2, CD8+ T cells were observed to consistently represent a higher proportion of the CD3+ T-cell component relative to CD4+ T cells, and this was true for both cells actively growing in the gel and for those escaping the gel (FIG. 20b). Over the 15-day time course, we observed that the proportion of CD4+ and CD8+ T cells remained stable in the media but that the proportion of encapsulated CD8+ T cells was increased over time. As control, we immunophenotyped these cells growing in the medium in absence of CTGel2 to observe that the higher overall proportion of the CD8+ T cell component is indeed a feature of IL-2-mediated T cell expansion from these different PBMC donors rather than an effect on their growth in presence of the gel (FIG. 20b, far right). These results indicate that the CTGel2, under these experimental conditions, is favorable to the proportional growth of CD8+ T cell populations over time, and this exemplifies the potential use of this formulation as a host for the growth of a 3D culture that may have true therapeutic potential against solid tumors.

T Cells and TIL Escape the Chitosan Thermogel Over Time:

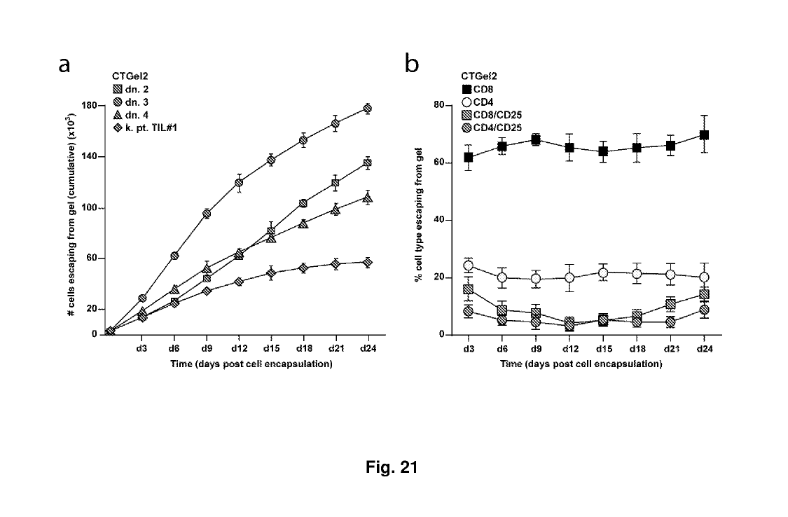

Our earlier results demonstrated that encapsulated cells could grow within and also escape the CTGel2 over time. From the observed steady state number of cells in the media, it was uncertain whether cells were truly escaping the gel or simply dividing in the media above the gel (FIG. 18b, medium). Therefore we performed 24-day experiments where every three days post cell-encapsulation the media above the CTGel2 encapsulated cells was completely collected for analysis using flow cytometry and was replaced with fresh media. We used the same three different normal donors (i.e., dn. 1, 2 and 3) and we also used TIL expanded from a kidney cancer patient tumor (i.e., k. pt. TIL#1) (FIG. 21a). Control conditions using the CTGel1 were also used to observe that this gel did not allow a high proportion of cells to escape the gel, as was observed from earlier experiments (data not shown). Over these longer time courses, the weights of CTGels were also recorded to verify that no loss in gel integrity was occurring. FIG. 21a results demonstrate that indeed, cells are continuously being released from the gel over time. From an initial encapsulation of 4×106 cells per 0.5 mL of CTGel2, as many as 1.8×105 T cells from normal donor 2 (i.e., dn. 2) were observed to escape from the gel over a period of 24 days. We expect the number of cells escaping the gel could be accelerated when encapsulated cells face more strongly stimulating inflammatory and chemoattractant signatures emanating from an adjacent tumor microenvironment.

In these experiments, we also immunophenotyped the cells using flow cytometry to observe their CD4+ and CD8+ proportions, along with their CD25+ activation status. We gated on morphology, singlets, alive, and CD3 to then record percentages of T cells that were either CD4+ or CD8+, and we then gated onto these to record the percentages of CD4+ and CD8+ T cells that were also activated based on CD25 positivity. As we saw in earlier experiments, we here have again observed that the CTGel2 is more permissive to the growth of CD8+ T cells (FIG. 21b). In addition, in the analysis of CD25 expression, we observed that it is more highly expressed by CD8+ T cells at day 3, is incrementally decreased until day 12, and then has a resurgence in expression until day 24 to levels similar to those at the time of cell encapsulation. In accordance with the finding that the CD4+ T cells do not have as high of an increase in CD25, and that their percentages are not increasing with time as are those of CD8+ T cells (known to be more responsive to the IL-2 from the surrounding media being continuously replenished), this finding substantiates the skewing of the CD8+/CD4+ population ratio under these experimental conditions. Altogether, these results indicate that the CTGel2 allows the continued escape of cells over time, and that this hydrogel formulation positively influences the phenotype and activation state of CD8+ T cells.

Encapsulated TIL are Activated and Escape in Response to Tumor Fragments:

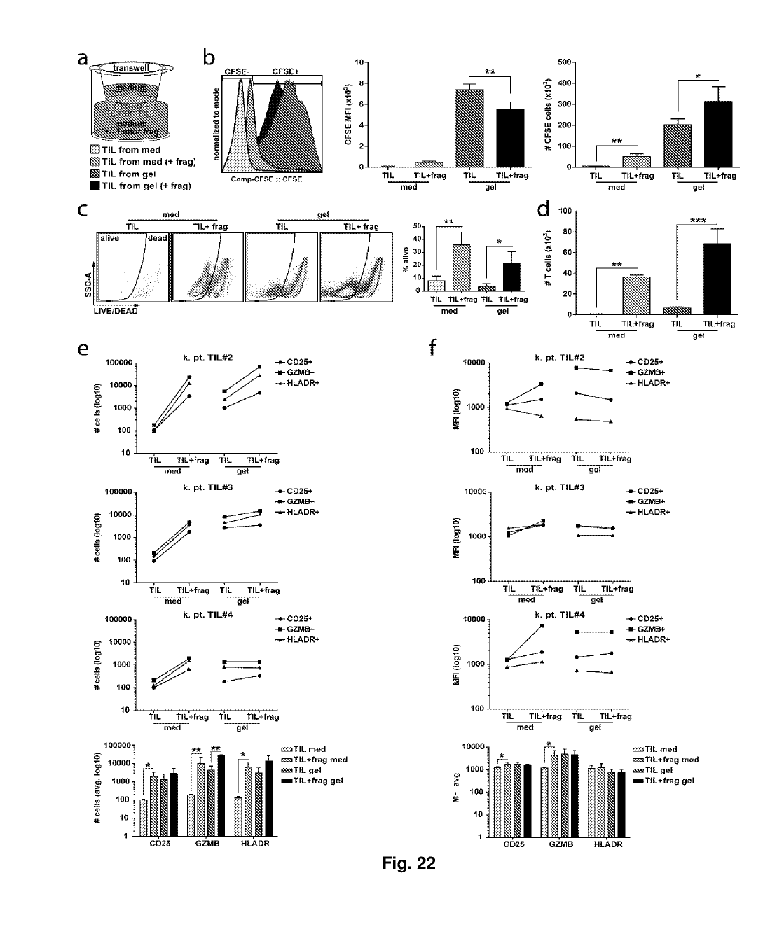

With the knowledge that T cells as well as TIL could grow in and escape the CTGel2 over time, and that the activation state of encapsulated T cells was influenced by surrounding conditions, we wondered if encapsulated TIL could also respond to tumors. We therefore applied a transwell system in order to challenge the CTGel2 encapsulated TIL with tumor fragments. Here, TIL expanded from resected ccRCC kidney tumors were CFSE labeled and encapsulated in CTGel2 before being poured in the transwell insert separating the encapsulated TIL from the tumor fragments placed in the bottom of the 24-well culture dishes below (FIG. 22a, illustration). The CFSE cell permeable fluorescent staining dye is covalently coupled to intracellular lysine residues, and is stable in cells over long periods of time. Its cell-derived fluorescence signal is diminished by each cell division, allowing the tracking of the labeled cells and their proliferation. Here, the CFSE labeling was important for both the tracking of CTGel encapsulated TIL proliferation, and the discerning of these from de novo expanding TIL from the living tumor fragments below. We collected all cells from the gel within the insert, and also from the cell culture dish medium below for analysis using flow cytometry five days following cell encapsulation.

We first analyzed cells according to their CFSE-fluorescence levels (FIG. 22b). Here, we observed the presence of CFSE-positive cells in the cell culture dish medium below in conditions where tumor fragments were also present (mean fluorescence intensity (MFI) 615±114), indicating that the tumors stimulated the escape of CTGel-encapsulated CFSE positive TIL. In the analysis of encapsulated cells, relative to conditions where no tumor fragments was present (MFI 7417±521), we observed a decrease in CFSE signal from the tumor fragment challenge conditions (MFI 5544±677), indicating that the presence of tumor fragments induce an increase in TIL cell division, and thus a diminishment in number of detectable CFSE molecules per cell (FIG. 22b, middle graph). Indeed, an increase in the numbers of CFSE positive encapsulated cells in tumor fragment-positive conditions (201.48K±29 vs 312.89K±76) is inversely correlated with their reduced CFSE signals as a result of increased cell division (FIG. 22b, right graph). Together, these results demonstrate that the tumor fragments caused an increase in TIL proliferation and migration towards the tumor environment.

Using live/dead staining and flow cytometry, gating on CFSE+/alive+ cells provided evidence that the total numbers of TIL that were alive in both the medium and the CTGel2 was >4-fold higher in presence if tumor fragments (FIG. 22c, dot plot and graph). By then gating on CD3, we observed that the presence of tumor fragments caused an increase in the number of TIL in both the CTGel2 and the medium in the wells below (i.e., from 6640±994 to 68385±14497 in gel, and from 124±21 to 36660±1590 in medium) from an initial 2×106 TIL encapsulated in 0.25 mL of CTGel2. Altogether, these results indicate that the presence of the tumors cause increases in TIL survival, growth, escape, and migration (FIG. 22d).

Finally, to verify that the cells growing and escaping in response to tumors where those that might be primed against the tumor, we analyzed cells that were CFSE+/CD3+ and also positive for early (CD25) and late (HLA-DR) activation, and cytotoxic Granzyme B (GZMB) markers [42-44]. Here, we analyzed both the numbers of cells positive for, and cell MFI for these markers (FIG. 22e&f). Across these patients, we observed that a consistently higher number of TIL from the medium were positive for these markers in conditions where the tumor fragment was present (FIG. 22e). As for TIL that had been CTGel2-encapsulated, only GZMB expression was significantly increased in conditions where tumor fragments were present (FIG. 22e). This indicates that in this system, TIL become more activated and potentially cytotoxic in response to tumor fragments as they come into closer contact with them. The MFI of CD25 and GZMB were also increased in the TIL analyzed from the medium, whereas no change in the MFI of these markers was observed for TIL extracted from gels (FIG. 22f). Altogether, these results demonstrate that in response to the tumor fragments they were originally expanded from, CTGel2-encapsulated TIL will increase in their proliferation and migration, and retain their capacities to express activation and cytotoxic markers.

Antigen-Specific Encapsulated T Cells Migrate to and Kill Cancer Cells:

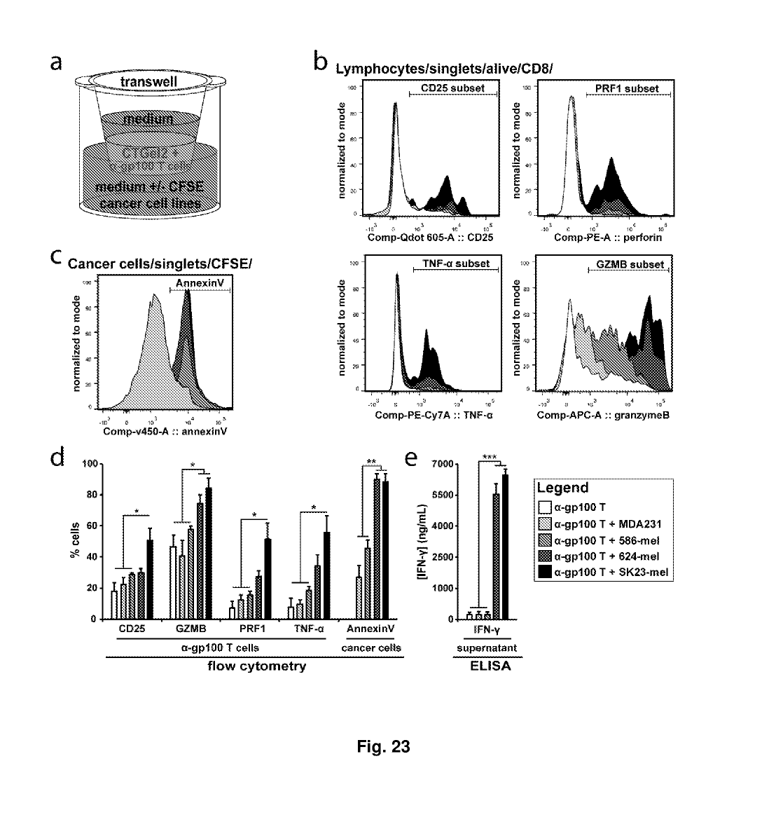

We then addressed whether the observed increases in activation and cytotoxic markers for encapsulated TIL were the result of their being challenged by their cognate target cancer cells. In addition, we wished to develop a robust assay for the in vitro and in vivo testing and inferring of the ability of encapsulated T cells to reduce tumor cell burden. Therefore, we adjoined the CTGel-transwell methodology to a common in vitro recognition assay, where a T cell clone specific to a melanoma antigen (gp100) presented by HLA-A2 was encapsulated in the CTGel2 poured in the transwell insert above, and the CFSE-labeled specific target melanoma cancer cells (SK23-mel and 624-mel, both HLA-A2+/gp100+) were seeded in the culture dishes below (FIG. 23a). The HLA-A2−/gp100+ 586-mel and breast cancer cell line MDA231 (HLA-A2+/gp100−) were used as negative controls. In this case, as CFSE is covalently bound, and thus retained by dead cells, its use would help us to identify target cells positive for apoptosis marker AnnexinV.

Cells were collected from the bottom well of the culture dish five days later, and flow cytometry was used to analyze the anti-gp100 T cells by first gating on lymphocytes/singlets/alive/CD8 to then analyze them for activation marker CD25, Th1 cytokine tumor necrosis factor-alpha (TNF-α), and cytotoxic markers Perforin-1 (PRF1) and GZMB (FIG. 23b). From these experiments, we consistently observed an increase in expression of these markers in the presence of their target cancer cells (and especially SK23-mel) (FIG. 23b&d). Flow cytometry was also used to analyze the AnnexinV content of the CFSE-labeled cancer cells, where we observed an increase in this marker in the target cancer cell lines 624-mel and SK23-mel, indicating that the anti-gp100 T cells that had escaped the CTGel2 and had migrated down to the bottom of the transwell system had also successfully killed a proportion of their target cells (FIG. 23c&d). Finally, ELISA was used to calculate the concentration of Th1 cytokine interferon gamma (IFNγ) from the cell supernatant, and revealed an increase in its expression in conditions where the target cancer cells were present, and which represents another readout of cancer cell killing efficacy of the encapsulated T cells (FIG. 23e). These results indicate that the CTGel2 encapsulated T cells escape the gel and migrate towards their target cancer cells, where they are activated, produce Th1 cytokine and cytotoxic markers, and also actually kill their target cells. This provides evidence that encapsulated T cells will retain their potential to kill the CTGel2-neighboring cancer cells of the tumor microenvironment.

### Conclusion

In all, our evidence supports the notion that the CTGel2, and very likely many other gels according to the invention, presents itself as an excellent support, unmatched in its class, towards the development of 3D TIL injectable cultures that will allows the encapsulated TIL to respond to and attack the nearby tumor environment. Other formulations of the hydrogel (namely using SHC+BGP) may also be favorable as injectable scaffold for T lymphocytes. In addition to T cell, other cell types such as B cells or dendritic cells or their combination could be encapsulated, in order to reproduce as close as possible the tertiary lymphoid tissue structure (TLS). Finally bioactive agent such as immune checkpoint inhibitors can be added in the gel to further enhance immunotherapy.

### Example 6

### Adding Another Biopolymer to the Gel:

In one embodiment, a gel containing CH with another biopolymer can be created, to further optimize the mechanical or biological properties. In this example, we show the feasibility of creating hydrogels with chondroitine sulfate.

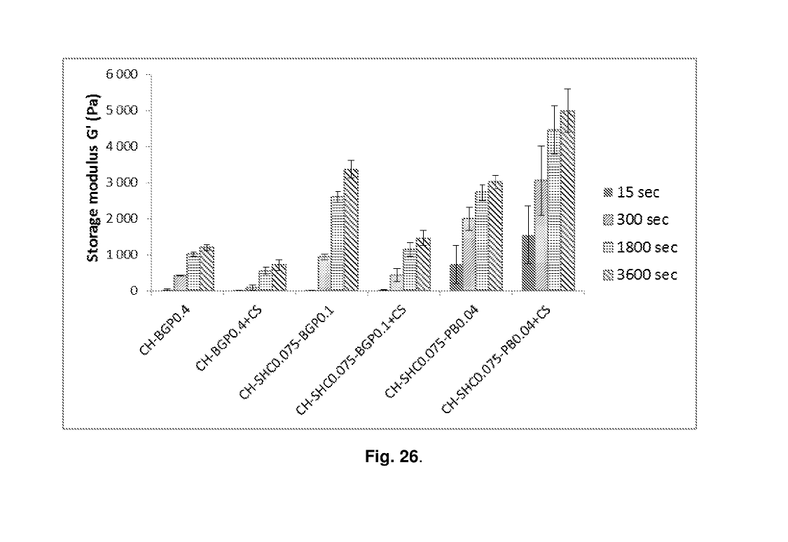

In an effort to optimize cell viability and growth within the hydrogel, Chondroitine sulfate (CS) was added within the gel by mixing it with the gelation agent solution to reach hydrogels with final concentrations from 0.125 to 1%. The pH of the hydrogels was measured at room temperature (RT) after mixing and after 24 h of gelation at 37° C. Rheological properties (within 1 h at 37° C.) were analyzed following the preparation of hydrogels. Elastic properties of the hydrogels were studied after 24 h of gelation at 37° C. using an ElastoSens™ system (Rheolution Inc). Direct cytotoxicity of the hydrogels was evaluated by entrapping L929 fibroblasts in the hydrogels and performing alamar blue and LIVE/DEAD assays. Indirect cytotoxicity was evaluated by studying the effect of hydrogel extracts on cell viability. All the formulations resulted in thermosensitive hydrogels with pH close to physiological levels and addition of CS did not disturb the pH balance. Addition of CS tend to decrease mechanical properties as assessed by ElastoSens, the storage modulus decreasing linearly as a function of CS concentration in the gel (FIG. 24). Even for 1% CS, G′ remained significantly higher than BGP hydrogels. CS strongly increased cell viability at 24 and growth at 7 compared to CH hydrogels, the effect being significant for CS concentrations of 0.5% and 1% (FIG. 25). Rheological properties were improved by CS (FIG. 26)

### Similarly Hydrogel Containing Heparin can be Created

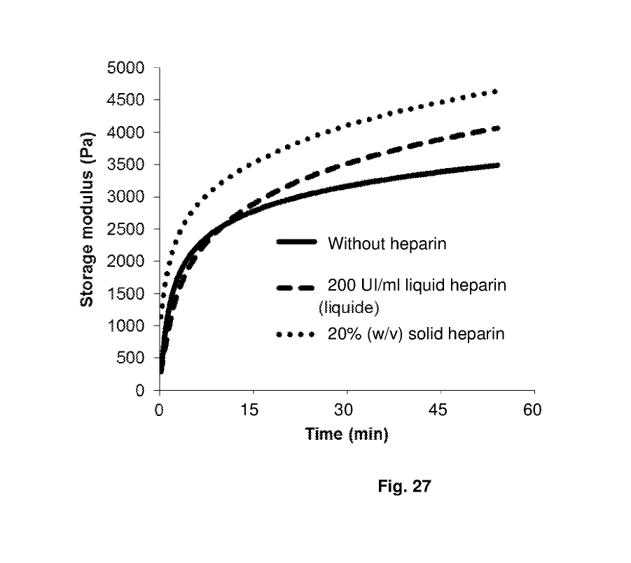

Heparin is a highly sulfated glycosaminoglycan which has interesting properties for biomedical applications. It can be used for its anticoagulant properties, to coat medical devices in contact with blood and avoid coagulation. It can also be used in hydrogel formulation for its anticoagulant properties, and for its ability to attract/bind growth factors in the cellular microenvironment and promote autocrine/paracrine activity of bioactive cells thus as mesenchymal stem cells or immune cells. Here, we have combined new chitosan hydrogels with heparin and have shown that their remain thermosensitive and keep their rapid gelation and high mechanical properties (FIG. 27).

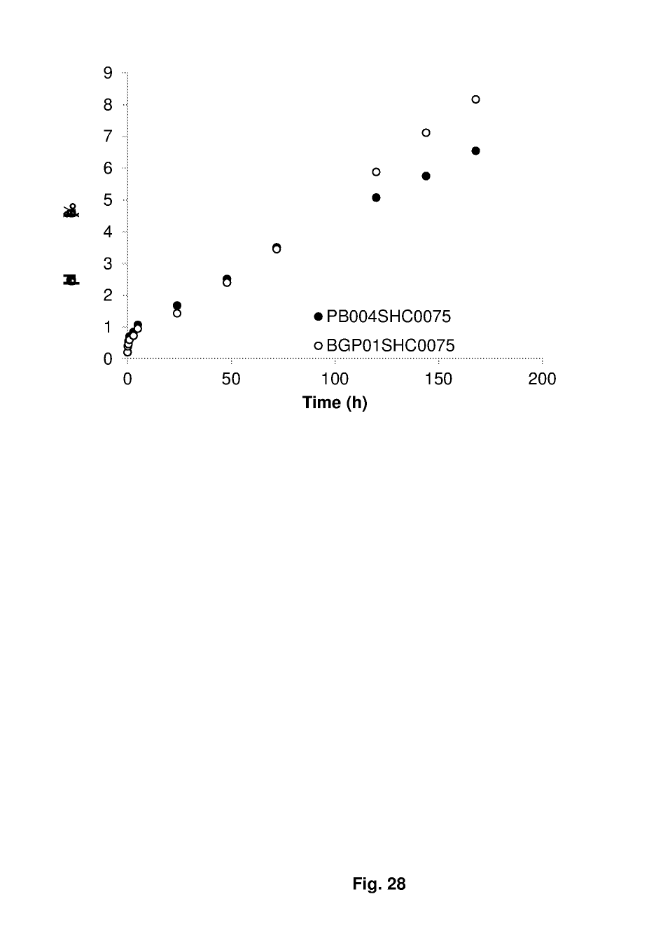

Heparin release from two chitosan gel (SHC0.075-PB0.04 and SHC0075BGP0.1) was studied by immersing hydrogels containing heparin (20%) in PBS. Heparin was quantified in the supernatant by Dimethylmethylene Blue over 160 hours. As presented in FIG. 28, heparin release (vertical axis in %) was relatively linear and sustain over the 160 hours.

### Example 7—Radiopaque and MRI Visible Injectable Hydrogels

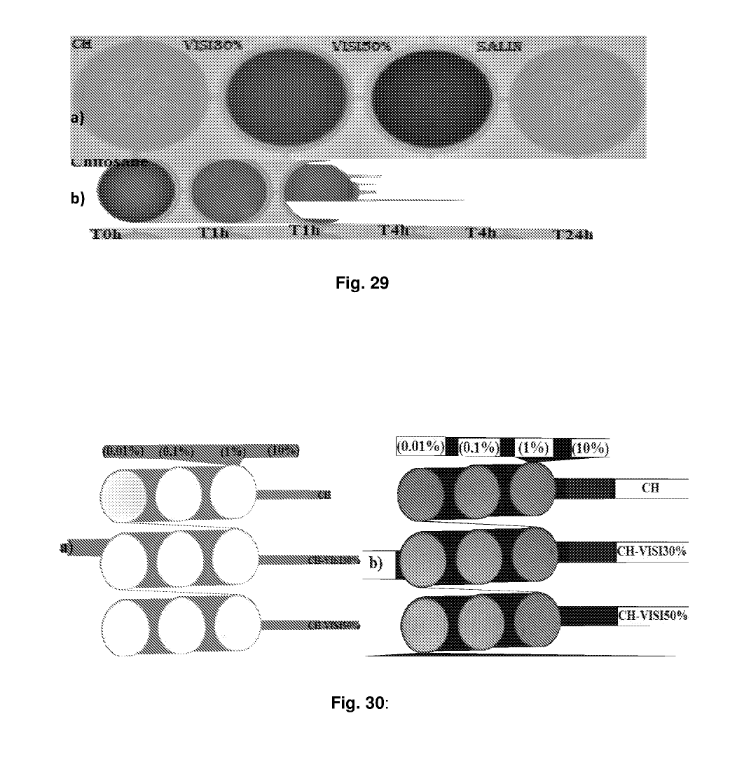

In another example, Visipaque® was added to hydrogels in order to make them radiopaque and visible by fluoroscopy during injection. Visipaque® is a contrast agent widely used in radiology and in interventional cardiology to opacity and observe blood vessels in fluoroscopy. Here, Visipaque® was combined with new chitosan hydrogel to localize it in vivo during endovasular administration and to deliver it at the desired site. Visipaque was added to the gel by replacing part of water using to prepare the acidic chitosan solution, at concentration reaching up to 50% v/v which gave it excellent radiopacity (FIG. 29A). The contrast agent is rapidly released from the hydrogel when immersed in a saline solution (FIG. 29B). Thus, it does not create artefact during follow-up imaging. A radiopaque hydrogel is particularly interesting for vascular applications.

Similarly a Gadolinium-rich contrast product was also added to the hydrogel to make it visible under MRI (Multihance 1% v/v). FIG. 30 shows the gel containing 0.01, 0.1, 1 and 10% Multihance observed during T1 and T2 sequence. Concentration between 0.1 and 1% were found to be optimal to obtain good contrast with surrounding tissues.

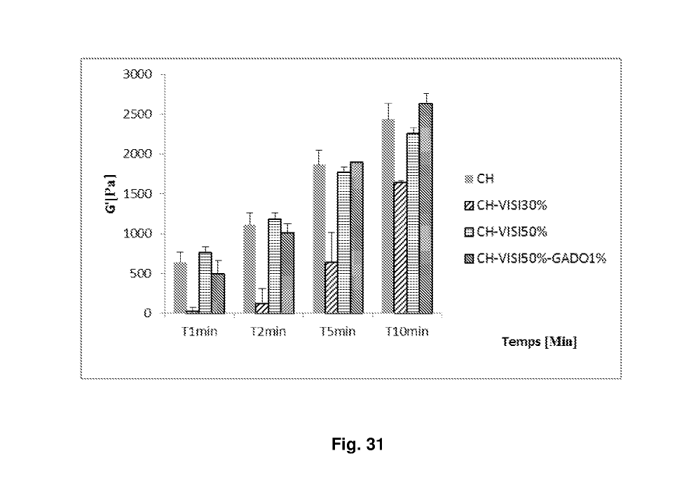

The addition of 50% Visipaque320 and 1% Multihance (Gadolinium) only slightly slow down gelation (FIG. 31) With some formulations such as SHC0075PB008, the initial G′ value was high, indicative of good initial mechanical properties, thus demonstrating the feasibility to create radiopaque hydrogels that could efficiently occlude the blood flow (FIG. 31).

### Example 8—Doxycycline-Loaded Hydrogels for Endoleak Treatment of Abdominal Aortic Aneurysms (AAA)

In another embodiment, a drug can be added to the gel, with or without the cells. For example, doxycycline, an antibiotic, can be mixed within the CH+GA and be released progressively to treat endoleaks and counter aneurysm progression. Doxycycline is known to exhibit sclerosing effects at high concentration and to be an inhibitor of metalloproteinase (MMPs) known to be involved in the progression of aneurysms. In this particular study, we aimed to achieve a initial burst release followed by slow release of the drug with time. CH gels were prepared by mixing a) a solution of CH (DDA=94%) dissolved in acidic solution containing contrast agent (iodixanol from GE Healthcare, USA) and b) a solution of gelation agent at ratio of 0.6:0.4 while CH-DOX gels were prepared by mixing (a) and 2 times concentrated of solution (b) followed by mixing with c) a solution containing DOX (Sigma) and anti-oxydants at 0.6:0.2:0.2 ratio.

Since X ray visibility is important to follow embolization in vivo, a iodide contrast agent (Visipaque) was also added in the gel (mixed in the CH acidic solution)

Direct addition in the CH or GA solution led to drug denaturation, as confirmed by spectrometry. In contrast, addition in the CH-GA solution at room temperature enabled to avoid drug denaturation and permitted homogeneous mixing. Addition of an anti-oxidant was however required for long term stability.

### Rheological Properties

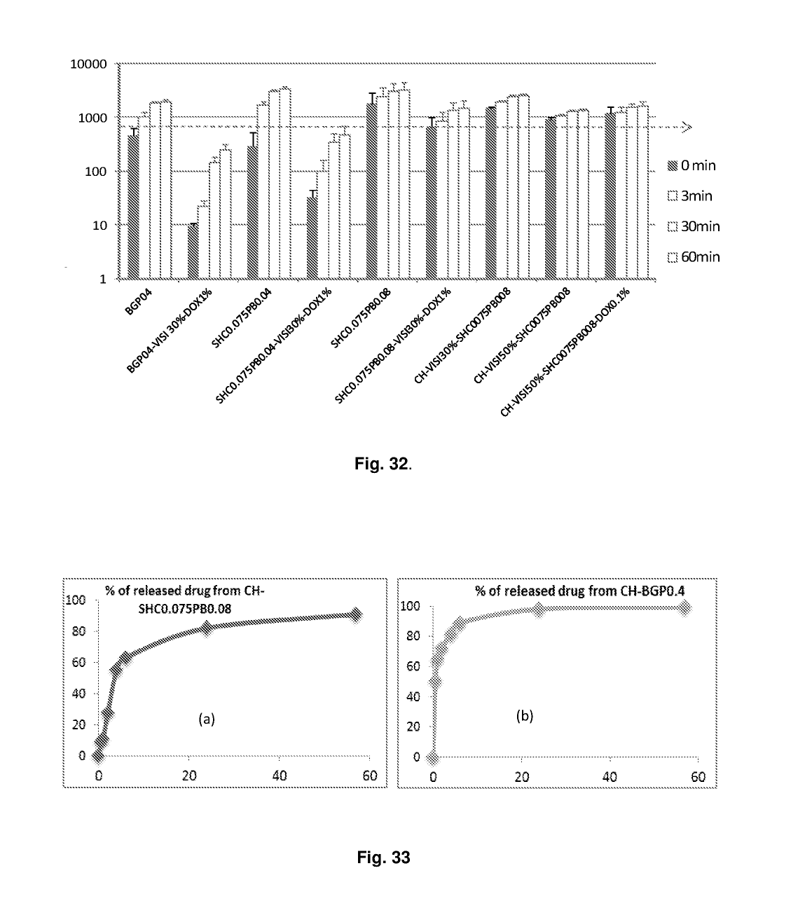

FIG. 32 presents the evolution of the storage modulus G′ in Pa (indicative of elastic properties) with time at 37° C. for CH hydrogels prepared with three different gelation agents (BGP0.4, SHC0.075PB0.04, SHC0.075PB0.08) with and without DOX (0.1 and 1%) and contrast agent (VISI (30 and 50%)). Results are expressed by the storage modulus (G)′ values at t=0, 3, 30 and 60 min. All the formulations gelled immediately at body temperature (G′>G″ at t=0). Addition of contrast agent and DOX was shown to slow down gelation. SHC0.075PB0.08 was found to exhibit better properties than SHC0075PB004 since G′ initial value was high (G′0=1190±339), even with DOX and Visipaque.

### In Vitro Release of Doxycycline

Drug release rate from hydrogels was evaluated using an USP apparatus II (by the Distek Tablet Dissolution Test equipment). The tests were run at 37° C. during 60 hours. Absorbance at 390 nm confirmed progressive drug release in PBS. The release of doxycycline follows a two-stage release, a burst release during the first 6 h, followed by a slow and continuous release from 6-60 hours. CH-SHC0.075PB0.08 gels slowed down the release compared to BGP04M counterparts (FIG. 33a). This two-stage release of the drug from hydrogel is appropriate for this application since the first phase is associated with the fast release of the majority of the drug to remove the endothelial cells while in the second stage, the remaining drug which is significantly less will be released from hydrogels in a longer time in order to inhibit MMPs derived from abdominal aortic aneurysms cells. In the case where a more linear release rate is expected, the drug could be loaded in microspheres before their addition into the gel.

### In Vitro Occlusive Properties and Injectability

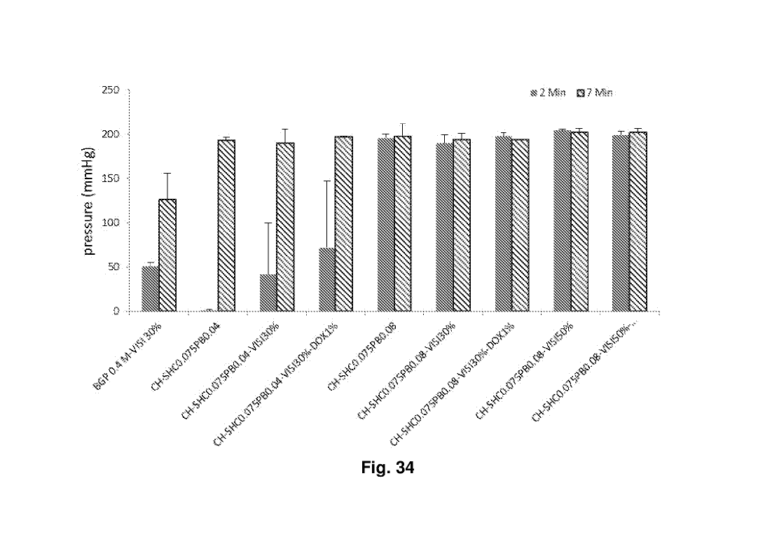

Occlusive properties of the gel was confirmed on an in vitro embolization bench test where the gel is injected in a tube which is then subject to flow at increasing pressure (up to a maximum of 220 mmHg). In vitro embolization tests confirmed that after 7 mins of gelation, all the formulations were able to sustain the maximum liquid pressure applied by the bench test. While gels prepared with BGP0.4 and SHC0.075PB0.04 even without contrast agent or DOX were not able to withstand maximal pressure after only 2 min of gelation, all gels prepared with SHC0.075PB0.08 did, even those containing VISI 50% and DOX 0.1% (FIG. 34).

### Ex Vivo Desendothelialization

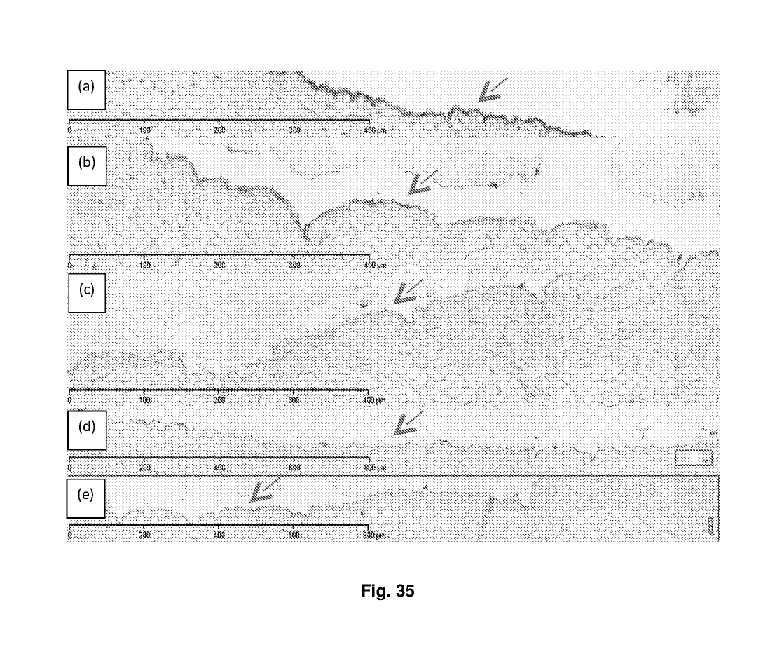

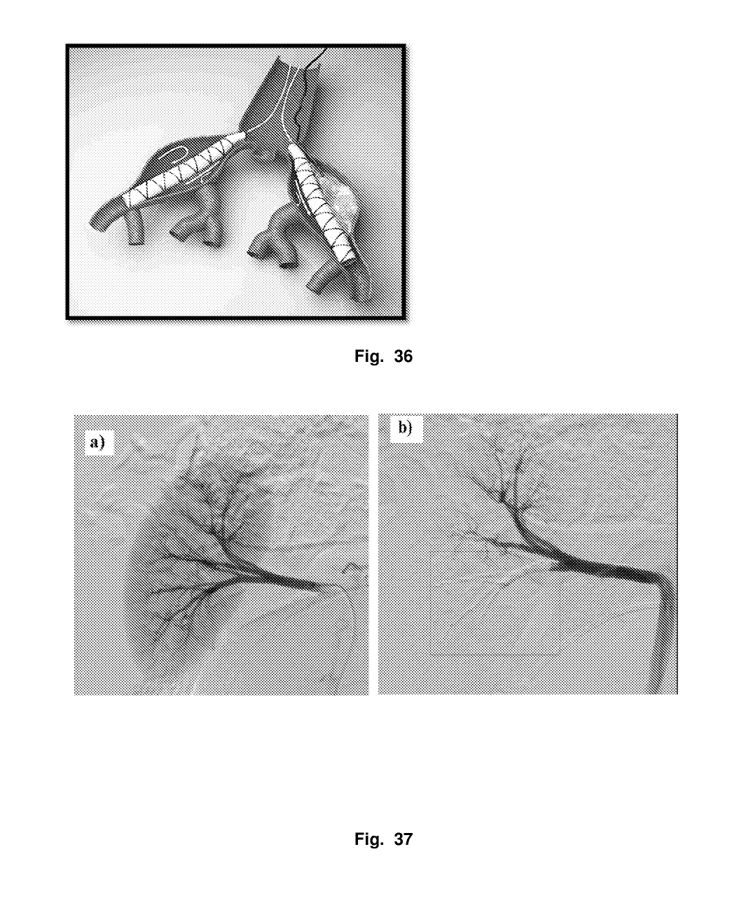

The effect of the gel on endothelial ablation was tested using ex vivo embolization of dog arteries. CH with DOX (0.1, 0.3 and 1% DOX final concentration) was compared to CH without DOX and control tissue (i.e. native tissue without gel injection). The embolized tissues were incubated at 37 C for 3 h, fixed, embedded in paraffin and factor VIII staining was done to visualize the endothelium (FIG. 35). FIG. 35a shows the intact layer of endothelial cells in the native arteries (brown layer). Injection of the CH gels without drug in the dog arteries slightly affected the endothelial cells layer (FIG. 35b) while injection of the CH gels including DOX at all the concentrations tested (i.e. 0.1, 0.3 and 1%) effectively removed the endothelial cells layer (FIG. 35 c-e). This can be an interesting strategy to provent or treat endoleaks after endovascular aneurysm repair (FIG. 36)

### In Vivo Testing

Preliminary in vivo embolization testing of the CH-SHC0075PB008-VISI50% and CH-SHC0075PB008-VISI50%-DOX0.1% gel was done by injection of the gels in the renal artery by microcatheter. Both gel formulations were easily injected with good control under fluoroscopy. Radiopacity was good and allowed real time monitoring of aneurysm embolization and also detection of gel migration when present. In both cases the arteries was completely blocked immediately after Injection and remained occluded for at least 2 h post-injection (FIG. 37).

### Example 9—In Situ Patch Formation and Prevention of Tissue Adhesion

In one embodiment, the injectable hydrogel can be used to prevent tissue adhesion after surgery

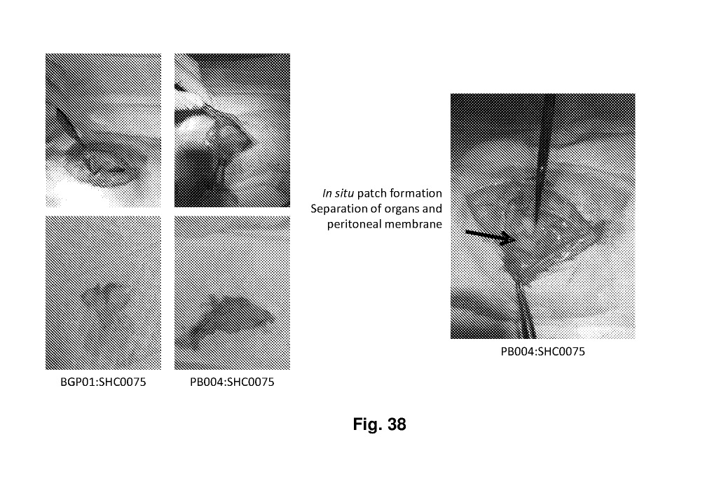

Adhesions are fibrous tissues that form between organs consecutively to an injury during clinical surgery. This scar tissue formation can be prevented if injured tissues are physically separated during the healing phase. New chitosan hydrogels are injectables and able to be spread on organs after injection. Their rapid gelation kinetic allows them to form a strong physical barrier. New chitosan thermogels were injected through a needle (20G+1″) in abdominal cavity of rats. After 10 minutes, abdominal cavity was reopened and chitosan thermogel formed a solid hydrogel barrier that separated organs (FIG. 38)

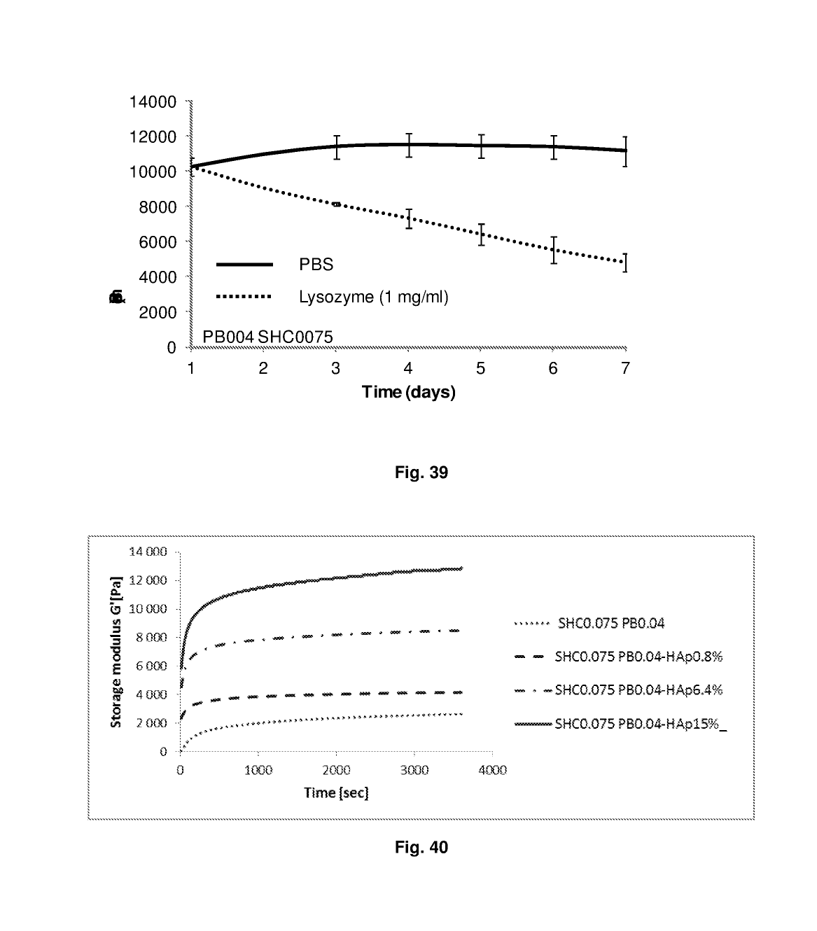

To verify the biodegradability of the hydrogel, After gelation, PB004:SH00075 hydrogel were immersed in phosphate buffer saline (PBS) or in a solution containing a physiological concentration of lysozyme. Lysozyme is an enzyme present in biological tissue and which is able to cleave chitosan and is responsible for its resorbable properties in vivo. Rheological properties decreased during immersion in lysozyme showing the preservation of resorbable properties of new formulations (FIG. 39, vertical axis is shear modulus in Pa).

### Example 10—Mineralized Hydrogels for Bone Application

In one embodiment, the injectable hydrogel can be filled with mineral particles and used for the regeneration of bone tissue.

In this example, hydroxyapatite particles are added in the gelation agent solution and mixed with the CH solution using two syringes connected through a luer lock as described in previous examples. Rheological properties were measured during gelation at 37 C for various concentrations of HAp in SHC0075-PB004 hydrogels. HAp did not prevent thermosensitive gelation and strongly increased the storage modulus at each time point compared to gels without HAp. (FIG. 40)

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can therefore be modified without departing from the spirit and nature of the subject invention as defined in the appended claims.

### REFERENCES

Ahmadi, R., & de Bruijn, J. D. (2008). Biocompatibility and gelation of chitosan-glycerol phosphate hydrogels. Journal of Biomedical Materials Research Part A, 86(3), 824-832.
Amrutkar, J., & Gattani, S. (2009). Chitosan-Chondroitin Sulfate Based Matrix Tablets for Colon Specific Delivery of Indomethacin. AAPS Pharm Sci Tech, 10(2), 670-677.
Bruedigam, C., et al., Basic techniques in human mesenchymal stem cell cultures: differentiation into osteogenic and adipogenic lineages, genetic perturbations, and phenotypic analyses. Curr Protoc Stem Cell Biol, 2011. Chapter 1: p. Unit1 H 3.
Chenite, A., Buschmann, M., Wang, D., Chaput, C., & Kandani, N. (2001). Rheological characterisation of thermogelling chitosan/glycerol-phosphate solutions. Carbohydrate Polymers, 46(1), 39-47.
Chenite, A., Chaput, C., Wang, D., Combes, C., Buschmann, M. D., Hoemann, C. D., et al. (2000). Novel injectable neutral solutions of chitosan form biodegradable gels in situ. Biomaterials, 21(21), 2155-2161.
Cho, J., Heuzey, M. C., Begin, A., & Carreau, P. J. (2005). Physical gelation of chitosan in the presence of beta-glycerophosphate: the effect of temperature. Biomacromolecules, 6(6), 3267-3275.
Cho, J., Heuzey, M. C., Bégin, A., & Carreau, P. J. (2006). Chitosan and glycerophosphate concentration dependence of solution behaviour and gel point using small amplitude oscillatory rheometry. Food Hydrocolloids, 20(6), 936-945.
Coutu, J. M., Fatimi, A., Berrahmoune, S., Soulez, G., & Lerouge, S. (2013). A new radiopaque embolizing agent for the treatment of endoleaks after endovascular repair: Influence of contrast agent on chitosan thermogel properties. Journal of Biomedical Materials Research Part B: Applied Biomaterials, 101B(1), 153-161.
Desai, E. S., Tang, M. Y., Ross, A. E., & Gemeinhart, R. A. (2012). Critical factors affecting cell encapsulation in superporous hydrogels. Biomedical Materials, 7(2), 024108.
Fatimi, A., Chabrot, P., Berrahmoune, S., Coutu, J.-M., Soulez, G., & Lerouge, S. (2012). A new injectable radiopaque chitosan-based sclerosing embolizing hydrogel for endovascular therapies. Acta Biomaterialia, 8(7), 2712-2721.
Goldberg, R. N., Kishore, N., & Lennen, R. M. (2002). Thermodynamic Quantities for the Ionization Reactions of Buffers. Journal of Physical and Chemical Reference Data, 31(2), 231-370.
Hirsch, C., & Haller, C. (2004). Effect of extracellular hypertonicity and alkalosis on endothelial-derived EA.hy 926 cells in vitro. European Journal of Medical Research, 9(2), 71-77.
Huang, Z., Yu, B., Feng, Q., & Li, S. (2011). Modification of an Injectable Chitosan Scaffold by Blending with NaHCO₃ to Improve Cytocompatibility. Polymers & polymer composites, 19(9), 781-787.
Kim, S., Nishimoto, S. K., Bumgardner, J. D., Haggard, W. O., Gaber, M. W., & Yang, Y. (2010). A chitosan/β-glycerophosphate thermo-sensitive gel for the delivery of ellagic acid for the treatment of brain cancer. Biomaterials, 31(14), 4157-4166.
Larkin, P. (2011). Infrared and Raman Spectroscopy, Principles and Spectral Interpretation (pp. 230).
Lavertu, M., Filion, D., & Buschmann, M. D. (2008). Heat-induced transfer of protons from chitosan to glycerol phosphate produces chitosan precipitation and gelation. Biomacromolecules, 9(2), 640-650.
Lehr, C. M., Bouwstra, J. A., Schacht, E. H., & Junginger, H. E. (1992). In vitro evaluation of mucoadhesive properties of chitosan and some other natural polymers. International Journal of Pharmaceutics, 78(1-3), 43-48.
Li, X., Kong, X., Zhang, J., Wang, Y., Shi, S., Guo, G., et al. (2011). A novel composite hydrogel based on chitosan and inorganic phosphate for local drug delivery of camptothecin nanocolloids. Journal of Pharmaceutical Sciences, 100(1), 232-241.
Liu, L., Tang, X., Wang, Y., & Guo, S. (2011). Smart gelation of chitosan solution in the presence of NaHCO₃ for injectable drug delivery system. International Journal of Pharmaceutics, 414(1-2), 6-15.
Mayo, D. W., Miller, F. A., & Hannah, R. W. (2003). Course Notes on the Interpretation of Infrared and Raman Spectra (pp. 596).
Murphy, M. B., K. Moncivais, and A. I. Caplan, Mesenchymal stem cells: environmentally responsive therapeutics for regenerative medicine. Exp Mol Med, 2013. 45: p. e54.
Muzzarelli, R. A. A. (1977). Chitin. Oxford: Pergamon Press.
Nair, L. S., Starnes, T., Ko, J.-W. K., & Laurencin, C. T. (2007). Development of Injectable Thermogelling Chitosan-Inorganic Phosphate Solutions for Biomedical Applications. Biomacromolecules, 8(12), 3779-3785.
Qian, R. Q., & Glanville, R. W. (2005). Methods for purifying chitosan. U.S. Pat. No. 6,896,809.
Ravi Kumar, M. N. V. (2000). A review of chitin and chitosan applications. Reactive and Functional Polymers, 46(1), 1-27.
Restifo, N. P., M. E. Dudley, and S. A. Rosenberg, Adoptive immunotherapy for cancer: harnessing the T cell response. Nat Rev Immunol, 2012. 12(4): p. 269-81.
Roberts, G. A. F. (1992). Chitin chemistry. London: The Macmillan Press Ltd.
Wang, K., & Liu, Q. (2014). Chemical structure analyses of phosphorylated chitosan. Carbohydrate Research, 386(0), 48-56.
Zhou, H. Y., Jiang, L. J., Cao, P. P., Li, J. B., & Chen, X. G. (2015). Glycerophosphate-based chitosan thermosensitive hydrogels and their biomedical applications. Carbohydrate Polymers, 117(0), 524-536.

1. A chitosan based gel solution which comprises:
from about 0.2% to about 4% w/v of chitosan;
from about 0.001 M to about 0.4 M of sodium hydrogen carbonate (SHC);
a weak base differing from the SHC; and
viable cells dispersed in the chitosan based gel solution;
wherein immediately after preparation, the chitosan based gel solution is flowable and becomes a gel after a gelation time, the gelation time being temperature dependent.

2. The chitosan based gel solution as defined in claim 1, wherein the chitosan is chemically modified prior preparation of the chitosan based gel solution to introduce small functional groups to the chitosan structure.

3. (canceled)

4. (canceled)

5. The chitosan based gel solution as defined in claim 1, wherein the SHC is between about 0.04 M and 0.1 M.

6. The chitosan based gel solution as defined in claim 1, wherein the weak base is beta-glycerophosphate at between about 0.001 M and about 0.5 M.

7. The chitosan based gel solution as defined in claim 1, wherein the weak base is phosphate buffer at between about 0.02 M and about 0.2 M.

8. The chitosan based gel solution as defined in claim 1, wherein the chitosan based gel solution includes an acid selected from the group consisting of: hydrochloric acid, acetic acid, propionic acid, citric acid, lactic acid, tartaric acid, malic acid, glycolic acid, ascorbic acid and combinations thereof.

9. (canceled)

10. (canceled)

11. (canceled)

12. The chitosan based gel solution as defined in claim 1, wherein the chitosan based gel solution has a pH between about 6.7 and about 7.5 after gelation.

13. The chitosan based gel solution as defined in claim 1, wherein the chitosan based gel solution has an osmolality of between 200 mOsm/L and 600 mOsm/L.

14. The chitosan based gel solution as defined in claim 1, comprising between about 1.5% and 2% w/v of chitosan.

15. (canceled)

16. (canceled)

17. The chitosan based gel solution as defined in claim 1, wherein the gelation time is less than 5 min at 37° C. and wherein the chitosan based gel solution remains a free flowing solution for at least 5 minutes after preparation at 20° C.

18. (canceled)

19. The chitosan based gel solution as defined in claim 1, further comprising at least one contrast product for improving contrast in an imaging modality.

20. (canceled)

21. (canceled)

22. The chitosan based gel solution as defined in claim 1, further comprising a therapeutic agent different from the viable cells for treating a predetermined pathology, the therapeutic agent remaining active for treating the predetermined pathology after gelation.

23. (canceled)

24. The chitosan based gel solution as defined in claim 22, wherein the therapeutic agent includes a bioactive agent selected from the group consisting of growth factors, fibroblast growth factor (FGF), epidermal growth factor (EGF), Platelet-derived growth factor (PDGF), transforming growth factor-β (TGFβ), insulin-like growth factor (IGF), angiopoietin-1 (ANGPT1), bone morphogenetic protein (BMP)-2, BMP-4, BMP-7, vascular endothelial growth factor (VEGF), platelet-derived growth factor (PDGF), interleukin-1 receptor antagonists, peptides, Link N peptide, anti-cancer drugs, doxorubicin, Irinotecan, antibiotics, doxycycline, or anti-inflammatory drugs, 5-aminosalicylic acid, interleukins (IL), IL2, IL4 and an immune checkpoint inhibitor selected from anti-CTLA-4, anti-PD-1, anti-CD160, anti-CD57, anti-CD244, anti-LAG-3, anti-CD272, anti-KLRG1, anti-CD26, anti-CD39, anti-CD73, anti-CD305, anti-TIGIT, anti-TIM-3, anti-VISTA, and anticoagulant.

25. The chitosan based gel solution as defined in claim 1, wherein the viable cells include cells selected from the group consisting of stem cells, induced pluripotent stem cells (iPS cells), progenitor cells, differentiated cells, bulge cells, mesenchymal stem cells, endothelial cells, epithelial cells, vascular smooth muscle cells, chondrocytes, osteoblasts, nucleus pulposus cells, CD8 T cells, CD4 T cells, NK cells, gamma delta T cells, B cells, dendritic cells, CAR T cells, and combinations thereof.

26. (canceled)

27. (canceled)

28. (canceled)

29. (canceled)

30. (canceled)

31. The chitosan based gel solution as defined in claim 1, further comprising at least one biopolymer excipient selected from the group consisting of collagen, chondroitin sulfate (CS), carboxymethylated starch (CMS), hyaluronic acid (HA), Heparin (Hep), extracellular matrix compounds and combinations thereof.

32. (canceled)

33. The chitosan based gel solution as defined in claim 1, wherein the chitosan based gel solution reaches, after gelation, a secant storage modulus of from about 10 kPa to about 150 kPa.

34.-50. (canceled)

51. A method for treating a mammal, the method comprising
delivering at a target location in the mammal the chitosan based gel solution; and
allowing the chitosan based gel solution to gel at the target location after delivery in the mammal.

52.-121. (canceled)

122. The chitosan based gel solution as defined in claim 1, wherein the viable cells include immune cells operative for performing local cancer immunotherapy.

123. The chitosan based gel solution as defined in claim 122, further comprising an anti-cancer drug.

124. The chitosan based gel solution as defined in claim 1, further comprising mineral particles selected from the group consisting of hydroxyapatite, calcium phosphates, bioactive glass, clay and silicates and combinations thereof.
